(19) United States
(12) Patent Application Publication
(10) Pub. No.: US 2025/0360149 A1
(43) Pub. Date: Nov. 27, 2025

(54) GPCR INHIBITORS AND USES THEREOF

(71) Applicant: GPCR Therapeutics, Inc., Seoul (KR)

(72) Inventors: Devki SUKHTANKAR, Fremont, CA (US); Juan José FUNG, San Jose, CA (US); Niña G. CACULITAN, Berkeley, CA (US); DongSeung SEEN, Seoul (KR); Eunhee KIM, Seoul (KR); Jae-Yeon JEONG, Seoul (KR); Piotr J. ZALICKI, Belmont, CA (US); Josephine M. CARDARELLI, San Carlos, CA (US)

(21) Appl. No.: 18/873,710

(22) PCT Filed: Jun. 9, 2023
(86) PCT No.: PCT/US2023/024983

§ 371 Date: Dec. 10, 2024

Related U.S. Application Data
(60) Provisional application No. 63/351,101, filed on Jun. 10, 2022.
(60) Provisional application No. 63/369,738, filed on Jul. 28, 2022.

(51) Int. Cl. A61K 31/675; A61K 31/138; A61K 31/395; A61K 38/18; A61K 40/50; A61P 35/00

(52) U.S. Cl. CPC A61K 31/675; A61K 31/138; A61K 31/395; A61K 38/18; A61K 40/50; A61P 35/00; A61K 2239/39

(57) ABSTRACT

This invention relates to methods and compositions directed to mobilizing a cell in a subject by blocking CXCR4, a beta-adrenergic receptor, a GPCR, or any combination thereof. In some embodiments, the cell is a stem cell. In some embodiments, the cell is an immune cell.

### CROSS-REFERENCE TO RELATED APPLICATION(S)

This application is a U.S. National Stage Application of International Application No. PCT/US2023/024983, filed on Jun. 9, 2023, which claims priority to U.S. Provisional Patent Application No. 63/351,101, filed on Jun. 10, 2022 and U.S. Provisional Patent Application No. 63/369,738, filed on Jul. 28, 2022. The contents of each of which are hereby incorporated by reference in their entirety.

### BACKGROUND OF THE INVENTION

The invention disclosed herein relates generally to the mobilization of stem cells and immune cells.

Bone marrow is highly innervated by the sympathetic nervous system. Traumatic stress in humans and rodent models have shown persistently elevated levels of norepinephrine, a ligand of beta-adrenergic receptors, which is associated with bone marrow dysfunction (Bible et al 2014, Bible et al 2015a, Bible et al 2015b). In a rat model of traumatic stress, daily administration of propranolol, a beta-adrenergic receptor inhibitor, was shown to restore bone marrow function and increase erythroid progenitor colony growth in response to anemia (Alamo et al 2017). In multiple myeloma patients, a 28 day cycle of propranolol administration shifted cell differentiation away from a myeloid bias to an upregulation of CD34+ stem cells and genes associated with this phenotype (Knight et al 2020). Thus, beta blockers appear to have potential to improve hematopoietic stem cell mobilization by restoring bone marrow function.

CXC Chemokine receptor 4 (CXCR4) belongs to the superfamily of G protein-coupled receptors (GPCR). Binding of the chemokine CXCL12 (also known as SDF-1) to its receptor CXCR4 plays an essential role in homing and retention of hematopoietic stem cells (HSC) in the bone marrow. Blocking the CXCL12/CXCR4 axis can elicit rapid mobilization of HSC from bone marrow to the peripheral blood (Domingues et al 2017). CXCR4 antagonists like Burixafor (also referred to as GPC-100 or TG-0054), as well as Plerixafor (also referred to as AMD3100 or Mozobil), have been used clinically in combination with granulocyte-colony stimulating factor (G-CSF) for hematopoietic stem cell mobilization and subsequent autologous stem cell transplant in non-Hodgkin's Lymphoma and multiple myeloma patients. However, typically, a G-CSF regimen involves repeated multi-day injections and is associated with adverse side effects like severe bone pain. However, successful ASCT in lymphoma and MM patients is often hindered by poor mobilization with at least 15% of patients failing to produce the target cell dose of >2× 106 CD34+ cells/kg required to proceed with ASCT (Olivieri et al 2012).

### BRIEF DESCRIPTION OF THE DRAWINGS

FIGS. 1A-1B show white blood cell (WBC) mobilization in in C57/BL6 and Balb/c mice (FIG. 1A) and time-dependent WBC count (FIG. 1B) after treatment with GPC100.

FIGS. 2A-2B show circulating WBC counts upon injection of vehicle and GPC100 or vehicle and AMD3100 (FIG. 2A) and injection of G-CSF and GPC100 or G-CSF and AMD3100 (FIG. 2B).

FIG. 3 shows data from six studies indicating that seven-day pretreatment with propranolol significantly increased GPC100-induced WBC count in peripheral blood compared to a seven-day vehicle pretreatment.

FIG. 4 shows data for mice that were administered GPC100 after propranolol pretreatment, showing that white blood cells were mobilized to the extent that was comparable to those that received G-CSF.

FIGS. 5A-5B show cumulative data of several experiments, showing a large variation in mobilization in white blood cells (FIG. 5A) and lymphocytes (FIG. 5B) from the combination treatment of G-CSF and AMD3100.

FIGS. 6A-6B shows data for G-CSF enhanced mobilization by GPC100 with or without propranolol pretreatment in WBC (FIG. 6A) and progenitor cells (FIG. 6B).

FIGS. 7A-7C show data pooled from four experiments indicating that when combined with G-CSF, GPC100 mobilized more WBC compared to AMD3100, with addition of propranolol not altering the overall WBC count for any groups (FIG. 7A), lymphocyte counts suggesting that propranolol enhanced their mobilization when added to the G-CSF and GPC100 combination (FIG. 7B), and distribution of WBC differential count showing that addition of propranolol increased lymphocyte trafficking into the peripheral blood (FIG. 7C).

FIGS. 8A-8D show enhancement in mobilization of white blood cells (FIG. 8A), lymphocytes (FIG. 8B), neutrophils (FIG. 8C), and monocytes (FIG. 8D) by propranolol pretreatment.

FIG. 9 shows data from three studies showing a significant increase in GPC100-induced mobilization after propranolol pretreatment.

FIGS. 10A-10D show data demonstrating an increase in mobilized white blood cells (FIG. 10A), lymphocytes (FIG. 10B), neutrophils (FIG. 10C), and monocytes (FIG. 10D) from propranolol pretreatment mainly due to lymphocytes, as opposed to an SOC regimen mainly mobilizing neutrophils.

FIG. 11 shows data demonstrating an increase in mobilized WBCs from propranolol pretreatment mainly due to lymphocytes, as opposed to an SOC regimen mainly mobilizing neutrophils.

FIG. 12 shows a large variation in the SOC group in two studies, with only SOC also resulting in a reduced number of platelets.

FIG. 13 shows a large variation in the SOC group in two studies, with only SOC also resulting in a reduced number of platelets.

FIG. 14 shows data for determination of hematopoietic stem cell mobilization with the dosing regimen by flow cytometry, with no significant difference observed from the standard of care for LSK Cells.

FIG. 15 shows data for determination of hematopoietic stem cell mobilization with the dosing regimen by flow cytometry, with no significant difference observed from the standard of care for Lin−CXCR4+ cells.

FIG. 16 shows data demonstrating that the addition of propranolol to the combination of G-CSF and GPC100 caused maximum mobilization of WBCs, with a significant increase in mobilization compared to SOC or G-CSF and GPC100 combination treatment.

FIGS. 17A-17C shows data demonstrating that the addition of propranolol to the combination of G-CSF and GPC100 caused maximum mobilization of neutrophils (FIG. 17A), lymphocytes (FIG. 17B), and monocytes (FIG. 17C), with a significant increase in mobilization compared to SOC or G-CSF and GPC100 combination treatment.

FIG. 18 shows that GPC100 in combination with propranolol increased lymphocytes, while GPC100 in combination with G-CSF increased Neutrophils.

FIGS. 19A-19B show that increased mobilized circulating WBCs (FIG. 19A) and Colony Formation Units (FIG. 19B) was observed in groups with G-CSF.

FIGS. 20A-20B show that the triple combination of G-CSF, GPC100, and propranolol resulted in the highest number of colony-forming units (FIG. 20A) and burst-forming units (FIG. 20B).

FIG. 21 is a schematic of a study focused on mobilization into the peripheral blood.

FIG. 22 shows data for GPC100-induced mobilization in white blood cells in mice.

FIG. 23 shows data for time-dependent GPC100-induced mobilization in white blood cells in mice.

FIGS. 24A-24C show that 7-day administration of propranolol enhanced GPC100 induced mobilization of white blood cells (FIG. 24A), lymphocytes (FIG. 24B), and neutrophils (FIG. 24C) but had no effect on blood counts when administered alone.

FIGS. 25A-25C show that Nadolol enhanced GPC100-induced mobilization of white blood cells (FIG. 25A), lymphocytes (FIG. 25B), and neutrophils (FIG. 25C).

FIGS. 26A-26C show that 7-day beta-blocker administration with a single GPC100 did not increase LSK and Lin−CXCR4+ cells.

FIGS. 27A-27C show that propranolol was observed to enhance GPC100-induced mobilization of white blood cells (FIG. 27A), lymphocytes (FIG. 27B), and neutrophils (FIG. 27C).

FIG. 28 shows that lymphocytes increased with GPC100 and beta blockers, while neutrophils increased with G-CSF+AMD3100.

FIGS. 29A-29B show combined data for fold increase in LSK cells (FIG. 29A) and Lin−CXCR4+ cells (FIG. 29B) upon administration of G-CSF, AMD3100, vehicle, propranolol, GPC100, and/or nadolol.

FIGS. 30A-30B show combined data for fold increase in LSK cells (FIG. 30A) and Lin−CXCR4+ cells (FIG. 30B) upon administration of G-CSF, AMD3100, vehicle, propranolol, and/or GPC100.

FIGS. 31A-31C show mobilization data for white blood cells (FIG. 31A), lymphocytes (FIG. 31B), and neutrophils (FIG. 31C) for the triple combination.

FIG. 32 shows data of white blood cell populations for the triple combination.

FIG. 33 is a schematic of the hematopoietic hierarchy

FIG. 34 is a schematic for a colony forming unit assay.

FIG. 35 shows data showing that the triple combination mobilized the highest number of progenitor cells.

FIGS. 36A-36B show data showing that the triple combination resulted in the highest number of colony-forming units (FIG. 36A) and burst-forming units (FIG. 36B).

FIG. 37 shows images of BFU-E colonies (left) and CFU-GM colonies (right) from G-CSF and GPC-100 combination treatment (top), and G-CSF+AMD3100 combination treatment (bottom).

FIGS. 38A-38B show data showing that the triple combination was associated with the maximum increase in circulating WBCs (FIG. 38A) as well as progenitor cells, measured by total colony forming units (FIG. 38B) compared to other drug groups.

FIGS. 39A-39B show total CFU after G-CSF combination treatments (FIG. 39A) and treatment with GPC100 with and without propranolol (FIG. 39B).

FIGS. 40A-40B show cumulative data from three studies regarding the effect of propranolol on GPC100-induced mobilization of white blood cells (FIG. 40A) and lymphocytes (FIG. 40B).

FIGS. 41A-41B show that propranolol enhanced GPC100 induced mobilization of white blood cells (FIG. 41A) and lymphocytes (FIG. 41B) comparable with SOC.

FIGS. 42A-42C show data from a study of GPC100, AMD3100 or G-CSF induced WBC mobilization of white blood cells (FIG. 42A), lymphocytes (FIG. 42B), and neutrophils (FIG. 42C) using a single agent.

FIGS. 43A-43B show data on the effect of propranolol on GPC100-induced mobilization in the absence or presence of G-CSF in comparison with standard of care (SOC) in WBC for study 4 (FIG. 43A) and study 5 (FIG. 43B).

FIGS. 44A-44B show data on the effect of propranolol on GPC100-induced mobilization in the absence or presence of G-CSF in comparison with standard of care (SOC) in lymphocytes for study 4 (FIG. 44A) and study 5 (FIG. 44B).

FIGS. 45A-45B show data on the effect of propranolol on GPC100-induced mobilization in the absence or presence of G-CSF in comparison with standard of care (SOC) in neutrophils for study 4 (FIG. 45A) and study 5 (FIG. 45B).

FIGS. 46A-46C show data for a comparison study of mobilization of white blood cells (FIG. 46A), lymphocytes (FIG. 46B), and neutrophils (FIG. 46C) between GPC100 and AMD3100.

FIGS. 47A-47C show data for the effect of propranolol on GPC100-induced mobilization of white blood cells (FIG. 47A), lymphocytes (FIG. 47B), and neutrophils (FIG. 47C) with or without G-CSF and a comparison with standard of care.

FIGS. 48A-48B show combined data from six studies showing that 7-day propranolol treatment prior to GPC100 results in significantly enhanced WBC (FIG. 48A) and lymphocyte (FIG. 48B) cell counts in peripheral blood compared to GPC100 alone.

FIGS. 49A-49B show that the standard of care regimen mobilized more WBCs (FIG. 49A) compared to the propranolol and GPC100 combination but the standard of care did not mobilize more lymphocytes than propranolol and GPC100 (FIG. 49B).

FIGS. 50A-50B show that the addition of propranolol to the G-CSF and GPC100 combination mobilized significantly more WBC (FIG. 50A) and lymphocytes (FIG. 50B) compared to the standard of care.

FIGS. 51A-51B show combined data from six studies showing that 7-day propranolol treatment prior to GPC100 results in significantly enhanced WBC (FIG. 51A) and lymphocyte (FIG. 51B) cell counts in peripheral blood compared to GPC100 alone.

FIGS. 52A-52B show that the standard of care regimen mobilized more WBCs (FIG. 52A) but not lymphocytes (FIG. 52B) compared to the propranolol and GPC100 combination.

FIGS. 53A-53B show that the addition of propranolol to the G-CSF and GPC100 combination mobilized significantly more WBC (FIG. 53A) and lymphocytes (FIG. 53B) compared to the standard of care for stem cell mobilization.

FIG. 54 shows the distribution of WBC differentials.

FIGS. 55A-55C show data for GPC100, AMD3100 or G-CSF induced WBC (FIG. 55A), lymphocyte (FIG. 55B), and neutrophil (FIG. 55C) mobilization.

FIGS. 56A-56C show data for a comparison study of mobilization of white blood cells (FIG. 56A), lymphocytes (FIG. 56B), and neutrophils (FIG. 56C) between GPC100 and AMD3100.

FIGS. 57A-57C show data showing the effect of propranolol on GPC100-induced mobilization of white blood cells (FIG. 57A), lymphocytes (FIG. 57B), and neutrophils (FIG. 57C) with or without G-CSF, in comparison with standard of care.

FIGS. 58A-58B show combined data from three studies showing that when combined with G-CSF, GPC100 was observed to mobilize significantly more WBCs (FIG. 58A) and lymphocytes (FIG. 58B), compared to AMD3100.

FIGS. 59A-59F shows that GPC100 inhibition of CXCR4 can be modulated by propranolol, in an in vitro activity assay (FIG. 59A), mobilization assay (FIG. 59B), co-localization of CXCR4 and B2AR in MDA-MB-231 cells expressing CXCR4 and B2AR (FIG. 59C) and control cells expressing CXCR4 alone (FIG. 59D), and Ca2+ flux assays in MDA-MB-231 inhibited with GPC-100 (FIG. 59E) or AMD3100 (FIG. 59F).

FIGS. 60A-60E show in vivo mobilization activity of GPC100 alone (FIG. 60A), propranolol followed by a dose of GPC100 or AMD3100 (FIG. 60B), and the effect of triple combination on mobilization (FIG. 60C), progenitor cells (FIG. 60D), and mouse HSCs (FIG. 60E).

FIGS. 61A-61C show an increase in WBC mobility in AMD3100 compared to vehicle in three studies (FIGS. 61A, 61B, and 61C).

FIG. 62 shows mobilization of hematopoietic stem cells as measured by LSK cells by flow cytometry.

FIGS. 63A-63B show WBC mobility when propranolol was administered by dose titration (5-40 mg/kg, IP) (FIG. 63A) and by pretreatment with propranolol (20 mg/kg, IP) over 7 days (FIG. 63B).

FIGS. 64A-64D show that phenotypic analyses for LSK cells by flow cytometry of vehicle (FIG. 64A), GPC-100 (FIG. 64B), and Propranolol with GPC-100 (FIG. 64C) also indicated that LSK cell mobilization (FIG. 64D) by GPC-100 was enhanced by propranolol.

FIG. 65 shows induction of mobilization by GPC-100 and propranolol combination compared with the standard of care, G-CSF.

FIG. 66 shows that WBC mobilization was significantly greater compared to the increased WBC count by G-CSF alone (4.5-fold) or G-CSF plus AMD3100 (6.6-fold).

FIG. 67A-67D shows total CFUs (CFU-GM+BFU) (FIG. 67A), BFUs (FIG. 67C), and both CFU-GM (clear bars) and BFUs (solid bars) (FIG. 67B) as well as WBC migration (FIG. 67D) after triple combination.

FIG. 68A-68F shows flow cytometry after treatment with vehicle (FIG. 68A); G-CSF and GPC-100 (FIG. 68D); G-CSF, propranolol, and GPC-100 (FIG. 68E); and G-CSF and AMD3100 (FIG. 68F); as well as MSC (FIG. 68B) and WBC (FIG. 68C) mobilization.

### ABBREVIATIONS

Unless indicated otherwise, the following includes abbreviations for terms disclosed herein: acute myeloid leukemia (AML), Adenosine A3 Receptor (ADORA3), Adenosine Receptor A2b (ADORA2B), adenovirus high-throughput system (AdHTS), Adenylate Cyclase Activating Polypeptide 1 (Pituitary) Receptor Type I (ADCYAP1R1), Adrenoceptor Alpha 1A (ADRA1A), Adrenoceptor Beta 2 (ADRB2), Apelin Receptor (APLNR), Atypical chemokine receptor 3 (ACKR3), bimolecular fluorescence complementation (BiFC), Bioluminescence Resonance Energy Transfer (BRET), bovine serum albumin (BSA), Calcitonin Receptor (CALCR), Cancer stem cells (CSCs), C-C chemokine receptor type 2 (CCR2), chemerin chemokine-like receptor 1 (CMKLR1), Cholinergic Receptor Muscarinic 1 (CHRM1), chronic lymphocytic leukemia (CLL), chronic myeloid leukemia (CML), chronic obstructive pulmonary disease (COPD), Complement C5a Receptor 1 (C5AR1), C-terminal fragment of Venus (VC), C-X-C Motif Chemokine ligand 12 (CXCL12), CXC receptor 4 (CXCR4), cytotoxic T-lymphocyte-associated antigen 4 (CTLA-4), 8-opioid receptor (OPRD), Endothelin Receptor Type B (EDNRB), enzyme-linked immunosorbent assay (ELISA), formalin-fixed paraffin-embedded (FFPE), fluorescence resonance energy transfer (FRET), G protein-coupled receptor (GPCR), Galanin Receptor 1 (GALR1), glioblastoma multiforme (GBM), Glucagon receptor (GCGR), GPCR heteromer identification technology (GPCR-HIT), Granulocyte-colony stimulating factor (G-CSF), hematopoietic stem cells (HSCs), hepatocellular carcinoma (HCC), Histamine Receptor H1 (HRH1), human immunodeficiency virus (HIV), International Union of Basic and Clinical Pharmacology Committee on Receptor Nomenclature and Drug Classification (NC-IUPHAR), u-opioid receptor (MOR), Motilin Receptor (MLNR), Multiple myeloma (MM), multiplicity of infection (MOI), Myelodysplastic Syndromes (MDS), Neurotensin Receptor 1 (NTSR1), non-Hodgkin lymphoma (NHL), non-small-cell lung cancer (NSCLC), N-terminal fragments of Venus (VN), patient derived cell (PDC), Patient-Derived Xenograft (PDX), positron emission tomography (PET), Computed Tomography (CT), programmed cell death ligand 1 (PD-L1), programmed cell death protein 1 (PD-1), Prostaglandin E Receptor 2 (PTGER2), Prostaglandin E Receptor 3 (PTGER3), proximity ligation assay (PLA), reverse transcription-quantitative polymerase chain reaction (RT-qPCR), Single-photon emission computed tomography (SPECT), small lymphocytic lymphoma (SLL), small-cell lung cancer (SCLC), Somatostatin Receptor 2 (SSTR2), Stromal cell-derived factor 1 (SDF-1), systemic lupus erythematosus (SLE), Tachykinin Receptor 3 (TACR3), Threshold cycles (Ct), time-resolved FRET (TR-FRET), tumor microenvironment (TME), Vascular endothelial growth factor (VEGF), vascular smooth muscle cells (VSMC), WHIM syndrome (Warts, Hypogammaglobulinemia, Infections, and Myelokathexis), green fluorescence protein (GFP), and yellow fluorescence protein (YFP).

### DETAILED DESCRIPTION OF THE INVENTION

Blood cells play a crucial part in maintaining the health and viability of animals, including humans. White blood cells, part of the body's immune system that help the body fight infection and other diseases, include granulocytes (neutrophils, eosinophils and basophils/mast cells), monocytes/macrophages, as well the lymphocytes (T and B cells) of the immune system. White blood cells are continuously replaced via the hematopoietic system, by the action of colony stimulating factor (CSF) and various cytokines on stem cells and progenitor cells in hematopoietic tissues.

One of the most widely known of these is granulocyte colony stimulating factor (G-CSF), which has been approved for use in counteracting the negative effects of chemotherapy by stimulating the production of white blood cells and progenitor cells (peripheral blood stem cell mobilization). See, e.g., U.S. Pat. No. 5,582,823, incorporated herein by reference, for the hematopoietic effects of G-CSF.

The development and maturation of blood cells is a complex process. Mature blood cells are derived from hematopoletic precursor (progenitor) cells and stem cells present in specific hematopoietic tissues including bone marrow. Within these environments hematopoietic cells proliferate and differentiate prior to entering the circulation.

The chemokine receptor CXCR4 and its natural ligand stromal cell derived factor-1 (SDF-1) appear to be important in this process (for reviews, see Maekawa, T., et al., Internal Med. (2000) 39:90-100; Nagasawa, T., et al., Int. J. Hematol. (2000) 72:408-411). This is demonstrated by reports that CXCR4 or SDF-1 knock-out mice exhibit embryonic lethality and hematopoietic defects (Ma, Q., et al., Proc. Natl. Acad. Sci USA (1998) 95:9448-9453; Tachibana, K., et al., Nature (1998) 393:591-594; Zou, Y-R., et al., Nature (1998) 393:595-599). It is known that CD34+ progenitor cells express CXCR4 and require SDF-1 produced by bone marrow stromal cells for chemoattraction and engraftment (Peled, A., et al., Science (1999) 283:845-848). It is also known that, in vitro, SDF-1 is chemotactic for both CD34+ cells (Aiuti, A., et al., J. Exp. Med. (1997) 185:111-120; Viardot, A., et al., Ann. Hematol. (1998) 77:194-197) and for progenitor/stem cells (Jo, D-Y., et al., J. Clin. Invest. (2000) 105:101-111). SDF-1 is also an important chemoattractant, signaling via the CXCR4 receptor, for several other more committed progenitors and mature blood cells including T-lymphocytes and monocytes (Bleul, C., et al., J. Exp. Med. (1996) 184:1101-1109), pro- and pre-B lymphocytes (Fedyk, E. R., et al., J. Leukoc. Biol. (1999) 66:667-673; Ma, Q., et al., Immunity (1999) 10:463-471) and megakaryocytes (Hodohara, K., et al., Blood (2000) 95:769-775; Riviere, C., et al., Blood (1999) 95:1511-1523; Majka, M., et al., Blood (2000) 96:4142-4151; Gear, A., et al., Blood (2001) 97:937-945; Abi-Younes, S., et al, Circ. Res. (2000) 86:131-138).

Thus, it appears that SDF-1 is able to control the positioning and differentiation of cells bearing CXCR4 receptors whether these cells are stem cells (i.e., cells which are CD34+) and/or progenitor cells (which, being either CD34+ or CD34−, can result in the formation of specified types of colonies in response to particular stimuli) or cells that are somewhat more differentiated.

Recently, considerable attention has been focused on the number of CD34+ cells mobilized in the pool of peripheral blood progenitor cells used for autologous stem cell transplantation. The CD34+ population is the component thought to be primarily responsible for the improved recovery time after chemotherapy and the cells most likely responsible for long-term engraftment and restoration of hematopoiesis (Croop, J. M., et al., Bone Marrow Transplantation (2000) 26:1271-1279). The mechanism by which CD34+ cells re-engraft may be due to the chemotactic effects of SDF-1 on CXCR4 expressing cells (Voermans, C. Blood, 2001, 97, 799-804; Ponomaryov, T., et al., J. Clin. Invest. (2000) 106:1331-1339). For example, adult hematopoietic stem cells were shown to be capable of restoring damaged cardiac tissue in mice (Jackson, K., et al., J. Clin. Invest. (2001) 107:1395-1402; Kocher, A., et al., Nature Med. (2001) 7:430-436). Thus, the role of the CXCR4 receptor in managing cell positioning and differentiation has assumed considerable significance.

As used herein, the term “progenitor cells” refers to cells that, in response to certain stimuli, can form differentiated hematopoietic or myeloid cells. The presence of progenitor cells can be assessed by the ability of the cells in a sample to form colony-forming units of various types, including, for example, CFU-GM (colony-forming units, granulocyte-macrophage); CFU-GEMM (colony-forming units, multipotential); BFU-E (burst-forming units, erythroid); HPP-CFC (high proliferative potential colony-forming cells); or other types of differentiated colonies which can be obtained in culture using known protocols.

As used herein, “stem” cells are less differentiated forms of progenitor cells. Typically, such cells are often positive for CD34. Some stem cells do not contain this marker, however. These CD34+ cells can be assayed using fluorescence activated cell sorting (FACS) and thus their presence can be assessed in a sample using this technique. In general, CD34+ cells are present only in low levels in the blood, but are present in large numbers in bone marrow. While other types of cells such as endothelial cells and mast cells also may exhibit this marker, CD34 is considered an index of stem cell presence.

The term “CXCR4” as used herein refers to C-X-C Motif Chemokine Receptor 4, also identified by unique database identifiers (IDs) and alternate names as shown in Table 1 (Chatterjee et al., 2014; Debnath et al., 2013; Domanska et al., 2013; Guo et al., 2016; Peled et al., 2012; Roccaro et al., 2014; Walenkamp et al., 2017). Table 1 also provides the nomenclature of CXCR4 and GPCRx that form heteromers with CXCR4 and synergistically enhance Ca2+ response upon co-stimulation with both agonists.

TABLE 1

| Gene name | Full name | Other names | IDs |
|---|---|---|---|
| CXCR4 | C-X-C Motif | Leukocyte-Derived Seven | GCID: GC02M136114 |
| | Chemokine | Transmembrane Domain | HGNC: 2561 |
| | Receptor 4 | Receptor; | Entrez Gene: 7852 |
| | | Lipopolysaccharide- | Ensembl: |
| | | Associated Protein 3; Stromal | ENSG00000121966 |
| | | Cell-Derived Factor 1 | OMIM: 162643 |
| | | Receptor; Chemokine (C-X- | UniProtKB: P61073 |
| | | C Motif) Receptor 4; LPS- | |
| | | Associated Protein 3; Seven | |
| | | Transmembrane Helix | |
| | | Receptor; C-X-C Chemokine | |
| | | Receptor Type 4; | |
| | | Neuropeptide Y Receptor | |
| | | Y3; Neuropeptide Y3 | |
| | | Receptor; Chemokine | |
| | | Receptor; Seven- | |
| | | Transmembrane-Segment | |
| | | Receptor, Spleen; Chemokine | |
| | | (C-X-C Motif), Receptor 4 | |
| | | (Fusin); SDF-1 Receptor; | |
| | | CD184 Antigen; Fusin; LAP- | |
| | | 3; LESTR; NPYRL; FB22; | |
| | | HM89; LCR1; D2S201E; | |
| | | HSY3RR; NPYY3R; | |
| | | CXC-R4; CXCR-4; CD184; | |
| | | NPY3R; WHIMS; LAP3; | |
| | | NPYR; WHIM | |
| ADCYAP1R1 | ADCYAP | Adenylate Cyclase | GCID: GC07P031058 |
| | Receptor Type I | Activating Polypeptide 1 | HGNC: 242 |
| | | (Pituitary) Receptor Type I; | Entrez Gene: 117 |
| | | Pituitary Adenylate Cyclase- | Ensembl: |
| | | Activating Polypeptide Type | ENSG00000078549 |
| | | 1 Receptor; PACAP Type I | OMIM: 102981 |
| | | Receptor; PACAP Receptor | UniProtKB: P41586 |
| | | 1; PACAP-R1; Pituitary | |
| | | Adenylate Cyclase | |
| | | Activating Polypeptide 1 | |
| | | Receptor Type I Hiphop; | |
| | | Pituitary Adenylate Cyclase- | |
| | | Activating Polypeptide Type | |
| | | I Receptor; PACAP-R-1; | |
| | | PACAPRI; | |
| | | PACAPR; PAC1R; PAC1 | |
| ADORA2B | Adenosine A2b | Adenosine Receptor A2b; | GCID: GC17P015927 |
| | Receptor | ADORA2 | HGNC: 264 |
| | | | Entrez Gene: 136 |
| | | | Ensembl: |
| | | | ENSG00000170425 |
| | | | OMIM: 600446 |
| | | | UniProtKB: P29275 |
| ADORA3 | Adenosine A3 | Adenosine Receptor A3; | GCID: GC01M111499 |
| | Receptor | A3AR | HGNC: 268 |
| | | | Entrez Gene: 140 |
| | | | Ensembl: |
| | | | ENSG00000282608 |
| | | | OMIM: 600445 |
| | | | UniProtKB: P0DMS8 |
| ADRB2 | Adrenoceptor | Adrenergic, Beta-2-, | GCID: GC05P148825 |
| | Beta 2 | Receptor, Surface; Beta-2 | HGNC: 286 |
| | | Adrenoreceptor; Beta-2 | Entrez Gene: 154 |
| | | Adrenoceptor; ADRB2R; | Ensembl: |
| | | B2AR; Adrenoceptor Beta 2, | ENSG00000169252 |
| | | Surface; Adrenoceptor Beta 2 | OMIM: 109690 |
| | | Surface; Beta-2 Adrenergic | UniProtKB: P07550 |
| | | Receptor; Catecholamine | |
| | | Receptor; | |
| | | BETA2AR; ADRBR; BAR | |
| C5AR1 | Complement | Complement Component 5a | GCID: GC19P047290 |
| | C5a Receptor 1 | Receptor 1; Complement | HGNC: 1338 |
| | | Component 5 Receptor 1 | Entrez Gene: 728 |
| | | (C5a Ligand); | Ensembl: |
| | | C5a Anaphylatoxin | ENSG00000197405 |
| | | Chemotactic Receptor 1; C5a | OMIM: 113995 |
| | | Anaphylatoxin Chemotactic | UniProtKB: P21730 |
| | | Receptor; | |
| | | Complement Component 5 | |
| | | Receptor 1; C5a | |
| | | Anaphylatoxin Receptor; | |
| | | C5a-R; | |
| | | C5R1; C5AR; CD88 | |
| | | Antigen; C5a Ligand; CD88; | |
| | | C5a | |
| CALCR | Calcitonin | CT-R; CTR1; CRT; CTR | GCID: GC07M093424 |
| | Receptor | | HGNC: 1440 |
| | | | Entrez Gene: 799 |
| | | | Ensembl: |
| | | | ENSG00000004948 |
| | | | OMIM: 114131 |
| | | | UniProtKB: P30988 |
| CHRM1 | Cholinergic | Acetylcholine Receptor, | GCID: GC11M062927 |
| | Receptor | Muscarinic 1; | HGNC: 1950 |
| | Muscarinic 1 | Muscarinic Acetylcholine | Entrez Gene: 1128 |
| | | Receptor M1; HM1; MIR; | Ensembl: |
| | | M | ENSG00000168539 |
| | | | OMIM: 118510 |
| | | | UniProtKB: P11229 |
| EDNRB | Endothelin | Endothelin Receptor Non- | GCID: GC13M077895 |
| | Receptor Type B | Selective Type; ET-BR; ET- | HGNC: 3180 |
| | | B; | Entrez Gene: 1910 |
| | | ETRB; | Ensembl: |
| | | Endothelin Receptor Subtype | ENSG00000136160 |
| | | B1; ABCDS; HSCR2; | OMIM: 131244 |
| | | ETB1; ETBR; WS4A; | UniProtKB: P24530 |
| | | HSCR; ETB | |
| HRH1 | Histamine | HH1R; H1R; Histamine | GCID: GC03P011113 |
| | Receptor H1 | Receptor, Subclass H1; | HGNC: 5182 |
| | | Histamine H1 Receptor; | Entrez Gene: 3269 |
| | | HisH1; H1-R | Ensembl: |
| | | | ENSG00000196639 |
| | | | OMIM: 600167 |
| | | | UniProtKB: P35367 |
| MLNR | Motilin Receptor | G Protein-Coupled Receptor | GCID: GC13P049220 |
| | | 38; GPR38; MTLR1; G- | HGNC: 4495 |
| | | Protein Coupled Receptor 38; | Entrez Gene: 2862 |
| | | MTLR | Ensembl: |
| | | | ENSG00000102539 |
| | | | OMIM: 602885 |
| | | | UniProtKB: O43193 |
| NTSR1 | Neurotensin | High-Affinity Levocabastine- | GCID: GC20P062708 |
| | Receptor 1 | Insensitive Neurotensin | HGNC: 8039 |
| | | Receptor; | Entrez Gene: 4923 |
| | | Neurotensin Receptor 1 | Ensembl: |
| | | (High Affinity); NT-R-1; | ENSG00000101188 |
| | | NTR1; NTRH; Neurotensin | OMIM: 162651 |
| | | Receptor Type 1; | UniProtKB: P30989 |
| | | NTRR; NTR | |
| TACR3 | Tachykinin | Neurokinin Beta Receptor; | GCID: GC04M103586 |
| | Receptor 3 | Neurokinin B Receptor; NK- | HGNC: 11528 |
| | | 3 Receptor; NK-3R; NK3R | Entrez Gene: 6870 |
| | | NKR; Neuromedin-K | Ensembl: |
| | | Receptor; | ENSG00000169836 |
| | | TAC3RL; TAC3R; HH11 | OMIM: 162332 |
| | | | UniProtKB: P29371 |

*GCID: Genecards identification
HGNC: HUGO Gene Nomenclature Committee

The terms “GPCRx” as used herein refers to GPCRs that were used in this study to investigate if these GPCRs interact with CXCR4 and show properties distinct from those of individual protomers, including ADCYAPIR1 (ADCYAP Receptor Type I), ADORA2B (Adenosine A2b Receptor), ADORA3 (Adenosine A3 Receptor), ADRB2 (Adrenoceptor Beta 2), APLNR (Apelin Receptor), C5AR1 (Complement C5a Receptor 1), CALCR (Calcitonin Receptor), CCR5 (Chemokine (C-C Motif) Receptor 5), CHRM1 (Cholinergic Receptor Muscarinic 1), GALR1 (Galanin Receptor 1), EDNRB (Endothelin Receptor Type B), HRH1 (Histamine Receptor H1), MLNR (Motilin Receptor), NTSR1 (Neurotensin Receptor 1), PTGER2 (Prostaglandin E Receptor 2), PTGER3 (Prostaglandin E Receptor 3), SSTR2 (Somatostatin Receptor 2), and TACR3 (Tachykinin Receptor 3), also identified by unique database identifiers (IDs) and alternate names as shown in Table 1.

The term “inhibitor” as used herein refers to molecule that inhibits or suppresses the enhanced function of a CXCR4, a beta-adrenergic receptor, a GPCR, a heteromer of CXCR4 and a beta-adrenergic receptor, and/or a CXCR4-GPCRx heteromer. Non-limiting examples of the inhibitor of the invention that can be used for mobilization of cells include GPCRx antagonist, GPCRx inverse agonist, GPCRx positive and negative allosteric modulator, CXCR4-GPCRx heteromer-specific antibody or its antigen binding portions including single-domain antibody-like scaffolds, bivalent ligands which have a pharmacophore selective for CXCR4 joined by a spacer arm to a pharmacophore selective for GPCRx, bispecific antibody against CXCR4 and GPCRx, radiolabeled CXCR4 ligand linked with GPCRx ligand, and small molecule ligands that inhibit heteromer-selective signaling. Certain examples of inhibitors against GPCRx that form heteromers with CXCR4 and enhance Ca2+ response upon co-stimulation with both agonists are listed in Table 2.

The term “antagonist” as used herein refers to a type of receptor ligand or drug that blocks or dampens a biological response by binding to and blocking a receptor, also called blockers. Antagonists have affinity but no efficacy for their cognate receptors, and their binding disrupts the interaction and inhibit the function of an agonist or inverse agonist at the cognate receptors. Certain examples of antagonists against GPCRx that form heteromers with CXCR4 and enhance Ca2+ response upon co-stimulation with both agonists are listed in Table 2.

TABLE 2
Examples of inhibitors against CXCR4 and ADRB2

| | | Antibodies/ |
|---|---|---|
| | | nanobodies/ |
| Gene name | Antagonists/Inverse agonists | i-bodies/others |
| CXCR4 | ALX40-4C, AMD070 (AMD11070, X4P-001), AMD3100 (plerixafor), | AD-114, AD-114- |
| | AMD3465, ATI 2341, BKT140 (BL-8040; TF14016; 4F-Benzoyl- | 6H, AD-114-Im7- |
| | TN14003), CTCE-9908, CX549, D-[Lys3] GHRP-6, FC122, FC131, GMI- | FH, AD-114- |
| | 1359, GSK812397, GST-NT21MP, isothiourea-1a, isothiourea-1t (IT1t), | PA600-6H, AD- |
| | KRH-1636, KRH-3955, LY2510924, MSX-122, N-[¹¹C]Methyl- | 214, ALX-0651, |
| | AMD3465, POL6326, SDF-1 1-9[P2G] dimer, SDF1 P2G, T134, T140, | LY2624587, PF- |
| | T22, TC 14012, TG-0054 (Burixafor), USL311, viral macrophage | 06747143, |
| | inflammatory protein-II (vMIP-II), WZ811, [⁶⁴Cu]-AMD3100, [⁶⁴Cu]- | ulocuplumab |
| | AMD3465, [⁶⁸Ga]pentixafor, [⁹⁰Y ]pentixather, [^{99m}Tc]O₂-AMD3100, | (MDX1338/BMS- |
| | [¹⁷⁷Lu]pentixather, and 508MCl (Compound 26). | 936564), 12G5, |
| | | 238D2, and 238D4 |
| ADRB2 | Alprenolol, atenolol, betaxolol, bupranolol, butoxamine, carazolol, | |
| | carvedilol, CGP 12177, cicloprolol, ICI 118551, ICYP, labetalol, | |
| | levobetaxolol, levobunolol, LK 204-545, metoprolol, nadolol, NIHP, NIP, | |
| | propafenone, propranolol, sotalol, SR59230A, and timolol. | |

The term “heteromer” as used herein refers to macromolecular complex composed of at least two GPCR units [protomers] with biochemical properties that are demonstrably different from those of its individual components. Heteromerization can be evaluated by in situ hybridization, immunohistochemistry, RNAseq, Reverse transcription-quantitative PCR (RT-qPCR, realtime PCR), microarray, proximity ligation assay (PLA), time-resolved FRET (TR-FRET), whole-body Single-photon emission computed tomography (SPECT) or Positron Emission Tomography/Computed Tomography (PET/CT).

The phrase “effective amount” as used herein refers to an amount sufficient to effect beneficial or desired results. An effective amount can be administered in one or more administrations, applications or dosages. Such delivery is dependent on a number of variables including the time period for which the individual dosage unit is to be used, the bioavailability of the agent, the route of administration, etc.

The phrase “therapeutically effective amount” as used herein refers to the amount of a therapeutic agent (e.g., an inhibitor, an antagonist, or any other therapeutic agent provided herein) which is sufficient to reduce, ameliorate, and/or prevent the severity and/or duration of a cancer and/or a symptom related thereto. A therapeutically effective amount of a therapeutic agent can be an amount necessary for the reduction, amelioration, or prevention of the advancement or progression of a cancer, reduction, amelioration, or prevention of the recurrence, development or onset of a cancer, and/or to improve or enhance the prophylactic or therapeutic effect of another therapy (e.g., a therapy other than the administration of a inhibitor, an antagonist, or any other therapeutic agent provided herein).

The phrase “therapeutic agent” refers to any agent that can be used in the treatment, amelioration, prevention, or management of a cancer and/or a symptom related thereto. In certain embodiments, a therapeutic agent refers to an inhibitor of CXCR4-GPCRx heteromer of the invention. A therapeutic agent can be an agent which is well known to be useful for, or has been or is currently being used for the treatment, amelioration, prevention, or management of a cancer and/or a symptom related thereto.

The phrase “intracellular Ca2+ assay,” “calcium mobilization assay,” or their variants as used herein refer to cell-based assay to measure the calcium flux associated with GPCR activation or inhibition. The method utilizes a calcium sensitive fluorescent dye that is taken up into the cytoplasm of most cells. The dye binds the calcium released from intracellular store and its fluorescence increases. The change in the fluorescence intensity is directly correlated to the amount of intracellular calcium that is released into cytoplasm in response to ligand activation of the receptor of interest.

The phrase “proximity-based assay” as used herein refers to biophysical and biochemical techniques that are able to monitor proximity and/or binding of two protein molecules in vitro (in cell lysates) and in live cells, including bioluminescence resonance energy transfer (BRET), fluorescence resonance energy transfer (FRET), bimolecular fluorescence complementation (BiFC), Proximity ligation assay (PLA), cysteine crosslinking, and co-immunoprecipitation (Ferre et al., 2009; Gomes et al., 2016).

Disclosed herein are methods and compositions directed to mobilizing a cell in a subject by blocking CXCR4, a beta-adrenergic receptor, a GPCR, or any combination thereof. In some embodiments, the cell is a stem cell. In some embodiments, the cell is an immune cell. In some embodiments, the mobilizing a cell in a subject comprises blocking CXCR4. In some embodiments, the mobilizing a cell in a subject comprises blocking a beta-adrenergic receptor. In some embodiments, the mobilizing a cell in a subject comprises blocking a GPCR. In some embodiments, the mobilizing a cell in a subject comprises blocking CXCR4 and a beta-adrenergic receptor. In some embodiments, the mobilizing a cell in a subject comprises blocking CXCR4 and a GPCR. In some embodiments, the mobilizing a cell in a subject comprises blocking a CXCR4-GPCR heteromer.

Disclosed herein are methods of mobilizing a cell in a subject, the method comprising: blocking CXCR4 signaling and beta-adrenergic receptor signaling in the subject. Also disclosed herein are methods of inducing cell mobilization in a subject, the method comprising: blocking CXCR4 signaling and beta-adrenergic receptor signaling in the subject. In embodiments, the blocking beta-adrenergic receptor signaling is performed before the blocking CXCR4 signaling. In some embodiments, the blocking beta-adrenergic receptor signaling is performed at a first specific time interval before the blocking CXCR4 signaling. In some embodiments, the first specific time interval is between 5 minutes to 10 minutes, 10 minutes to 20 minutes, 20 minutes to 30 minutes, 30 minutes to 40 minutes, 40 minutes to 50 minutes, 50 minutes to 1 hour, 1 hour to 2 hours, 2 hours to 3 hours, 3 hours to 4 hours, 4 hours to 5 hours, 5 hours to 6 hours, 6 hours to 12 hours, 12 hours to 24 hours, 1 day to 2 days, 2 days to 3 days, 3 days to 4 days, 4 days to 5 days, 5 days to 6 days, 6 days to 7 days, 7 days to 8 days, 8 days to 9 days, 9 days to 10 days, 10 days to 11 days, 11 days to 12 days, 12 days to 13 days, 13 days to 14 days, or 14 days or more. In embodiments, the blocking beta-adrenergic receptor signaling continues after the blocking CXCR4 signaling is terminated. In some embodiments, the blocking beta-adrenergic receptor signaling continues for a second specific time interval after the blocking CXCR4 signaling is terminated. In some embodiments, the second specific time interval is between 5 minutes to 10 minutes, 10 minutes to 20 minutes, 20 minutes to 30 minutes, 30 minutes to 40 minutes, 40 minutes to 50 minutes, 50 minutes to 1 hour, 1 hour to 2 hours, 2 hours to 3 hours, 3 hours to 4 hours, 4 hours to 5 hours, 5 hours to 6 hours, 6 hours to 12 hours, 12 hours to 24 hours, 1 day to 2 days, 2 days to 3 days, 3 days to 4 days, 4 days to 5 days, 5 days to 6 days, 6 days to 7 days, 7 days to 8 days, 8 days to 9 days, 9 days to 10 days, 10 days to 11 days, 11 days to 12 days, 12 days to 13 days, 13 days to 14 days, or 14 days or more.

In embodiments, the blocking CXCR4 signaling comprises administering a CXCR4 inhibitor to the subject.

In embodiments, the blocking beta-adrenergic receptor signaling comprises administering a beta-adrenergic receptor inhibitor to the subject. In embodiments, the blocking CXCR4 signaling comprises administering a CXCR4 inhibitor to the subject and the blocking beta-adrenergic receptor signaling comprises administering a beta-adrenergic receptor inhibitor to the subject. In embodiments, the cell is a stem cell. In some embodiments, the cell is an immune cell.

Disclosed herein are methods of mobilizing a stem cell in a subject, the method comprising: administering a beta-adrenergic receptor inhibitor and a CXCR4 inhibitor to the subject. Also disclosed herein are methods of inducing stem cell mobilization in a subject, the method comprising: administering a beta-adrenergic receptor inhibitor and a CXCR4 inhibitor to the subject. In some embodiments, the administering the beta-adrenergic receptor inhibitor is performed before the administering the CXCR4 inhibitor. In some embodiments, the administering the beta-adrenergic receptor inhibitor is performed at a first specific time interval before the administering the CXCR4 inhibitor. In some embodiments, the first specific time interval is between 5 minutes to 10 minutes, 10 minutes to 20 minutes, 20 minutes to 30 minutes, 30 minutes to 40 minutes, 40 minutes to 50 minutes, 50 minutes to 1 hour, 1 hour to 2 hours, 2 hours to 3 hours, 3 hours to 4 hours, 4 hours to 5 hours, 5 hours to 6 hours, 6 hours to 12 hours, 12 hours to 24 hours, 1 day to 2 days, 2 days to 3 days, 3 days to 4 days, 4 days to 5 days, 5 days to 6 days, 6 days to 7 days, 7 days to 8 days, 8 days to 9 days, 9 days to 10 days, 10 days to 11 days, 11 days to 12 days, 12 days to 13 days, 13 days to 14 days, or 14 days or more. In embodiments, the administering the beta-adrenergic receptor inhibitor continues after the administering the CXCR4 inhibitor is terminated. In some embodiments, the administering the beta-adrenergic receptor inhibitor continues for a second specific time interval after the administering the CXCR4 inhibitor is terminated. In some embodiments, the second specific time interval is between 5 minutes to 10 minutes, 10 minutes to 20 minutes, 20 minutes to 30 minutes, 30 minutes to 40 minutes, 40 minutes to 50 minutes, 50 minutes to 1 hour, 1 hour to 2 hours, 2 hours to 3 hours, 3 hours to 4 hours, 4 hours to 5 hours, 5 hours to 6 hours, 6 hours to 12 hours, 12 hours to 24 hours, 1 day to 2 days, 2 days to 3 days, 3 days to 4 days, 4 days to 5 days, 5 days to 6 days, 6 days to 7 days, 7 days to 8 days, 8 days to 9 days, 9 days to 10 days, 10 days to 11 days, 11 days to 12 days, 12 days to 13 days, 13 days to 14 days, or 14 days or more.

In embodiments, the beta-adrenergic receptor inhibitor is an ADRB2 inhibitor. In embodiments, the beta-adrenergic receptor inhibitor is selected from the group consisting of alprenolol, atenolol, betaxolol, bupranolol, butoxamine, carazolol, carvedilol, CGP 12177, cicloprolol, ICI 118551, ICYP, labetalol, levobetaxolol, levobunolol, LK 204-545, metoprolol, nadolol, NIHP, NIP, propafenone, propranolol, sotalol, SR59230A, and timolol. In embodiments, the beta-adrenergic receptor inhibitor is selected from the group consisting of propranolol, nadolol, and ICI 118551. In embodiments, the beta-adrenergic receptor inhibitor is propranolol.

In embodiments, the CXCR4 inhibitor is selected from the group consisting of ALX40-4C, AMD070 (AMD11070, X4P-001), AMD3100 (plerixafor), AMD3465, ATI 2341, BKT140 (BL-8040; TF14016; 4F-Benzoyl-TN14003), CTCE-9908, CX549, D-[Lys3] GHRP-6, FC122, FC131, GMI-1359, GSK812397, GST-NT21MP, isothiourea-la, isothiourea-1t (IT1t), KRH-1636, KRH-3955, LY2510924, MSX-122, N-[11C] Methyl-AMD3465, POL6326, SDF-1 1-9 [P2G] dimer, SDF1 P2G, T134, T140, T22, TC 14012, TG-0054 (Burixafor), USL311, viral macrophage inflammatory protein-II (vMIP-II), WZ811, [64Cu]-AMD3100, [64Cu]-AMD3465, [68Ga] pentixafor, [90Y] pentixather, [99mTc] 02-AMD3100, [177Lu] pentixather, and 508MC1 (Compound 26). Burixafor is also referred to as GPC-100 or TG-0054. Plerixafor is also referred to as AMD3100 or Mozobil. In embodiments, the CXCR4 inhibitor is selected from the group consisting of AD-214, AMD070 (AMD11070, X4P-001), AMD3100 (plerixafor), BKT140 (BL-8040; TF14016; 4F-Benzoyl-TN14003), CTCE-9908, LY2510924, LY2624587, T140, TG-0054 (Burixafor), PF-06747143, POL6326, and ulocuplumab (MDX1338/BMS-936564). In embodiments, the CXCR4 inhibitor is TG-0054 (burixafor). In embodiments, the CXCR4 inhibitor is AMD3100 (plerixafor). In embodiments, the CXCR4 inhibitor is ulocuplumab (MDX1338/BMS-936564).

In embodiments, the administering the CXCR4 inhibitor to the subject comprises administering TG-0054 (burixafor) and propranolol. In embodiments, the administering the CXCR4 inhibitor to the subject comprises administering AMD3100 (plerixafor) and propranolol. In embodiments, the administering the CXCR4 inhibitor to the subject comprises administering ulocuplumab (MDX1338/BMS-936564) and propranolol.

In embodiments, the method further comprises administering G-CSF to the subject. In embodiments, the administering the beta-adrenergic receptor inhibitor and the CXCR4 inhibitor to the subject is performed in the absence of G-CSF. Disclosed herein are methods of mobilizing a stem cell in a subject, the method comprising: administering a CXCR4 inhibitor and G-CSF to the subject, in the absence of a beta-adrenergic receptor inhibitor. Also disclosed herein are methods of inducing stem cell mobilization in a subject, the method comprising: administering a CXCR4 inhibitor and G-CSF to the subject, in the absence of a beta-adrenergic receptor inhibitor. In some embodiments, the administering the CXCR4 inhibitor to the subject comprises administering TG-0054 (burixafor) and propranolol. In embodiments, the administering the CXCR4 inhibitor to the subject comprises administering AMD3100 (plerixafor) and propranolol. In embodiments, the administering the CXCR4 inhibitor to the subject comprises administering ulocuplumab (MDX1338/BMS-936564) and propranolol.

In embodiments, the administering a combination of the CXCR4 inhibitor and the G-CSF induces an enhanced amount of cell mobilization relative to the amount of cell mobilization induced by the CXCR4 inhibitor only. In embodiments, the administering a combination of the CXCR4 inhibitor and the G-CSF mobilizes a cell by an amount enhanced relative to the amount of cell mobilization induced by the CXCR4 inhibitor only. In some embodiments, the enhanced amount of cell mobilization relative to the amount of cell mobilization induced by the CXCR4 inhibitor only is between 1.1-fold to 1.2-fold, 1.2-fold to 1.3-fold, 1.3-fold to 1.4-fold, 1.4-fold to 1.5-fold, 1.5-fold to 1.6-fold, 1.6-fold to 1.7-fold, 1.7-fold to 1.8-fold, 1.8-fold to 1.9-fold, 1.9-fold to 2-fold, 2-fold to 2.5-fold, 2.5-fold to 3-fold, 3-fold to 4-fold, 4-fold to 5-fold, 5-fold to 10-fold, or 10-fold or more. In some embodiments, the enhanced amount of cell mobilization relative to the amount of cell mobilization induced by the CXCR4 inhibitor only is between 5%-10% more, 10%-20% more, 20%-30% more, 30%-40% more, 40%-50% more, 50%-60% more, 60%-70% more, 70%-80% more, 80%-90% more, 90%-100% more, 100%-120% more, 120%-140% more, 140%-160% more, 160%-180% more, 180%-200% more, 200%-250% more, 250%-300% more, 300%-400% more, 400%-500% more, 500%-750% more, 750%-1000% more, or 1000% or more.

In embodiments, the administering a combination of the CXCR4 inhibitor and the beta-adrenergic receptor inhibitor induces an enhanced amount of cell mobilization relative to the amount of cell mobilization induced by the CXCR4 inhibitor only. In embodiments, the administering a combination of the CXCR4 inhibitor and the beta-adrenergic receptor inhibitor mobilizes a cell by an amount enhanced relative to the amount of cell mobilization induced by the CXCR4 inhibitor only. In some embodiments, the enhanced amount of cell mobilization relative to the amount of cell mobilization induced by the CXCR4 inhibitor only is between 1.1-fold to 1.2-fold, 1.2-fold to 1.3-fold, 1.3-fold to 1.4-fold, 1.4-fold to 1.5-fold, 1.5-fold to 1.6-fold, 1.6-fold to 1.7-fold, 1.7-fold to 1.8-fold, 1.8-fold to 1.9-fold, 1.9-fold to 2-fold, 2-fold to 2.5-fold, 2.5-fold to 3-fold, 3-fold to 4-fold, 4-fold to 5-fold, 5-fold to 10-fold, or 10-fold or more. In some embodiments, the enhanced amount of cell mobilization relative to the amount of cell mobilization induced by the CXCR4 inhibitor only is between 5%-10% more, 10%-20% more, 20%-30% more, 30%-40% more, 40%-50% more, 50%-60% more, 60%-70% more, 70%-80% more, 80%-90% more, 90%-100% more, 100%-120% more, 120%-140% more, 140%-160% more, 160%-180% more, 180%-200% more, 200%-250% more, 250%-300% more, 300%-400% more, 400%-500% more, 500%-750% more, 750%-1000% more, or 1000% or more.

In embodiments, the administering a combination of the CXCR4 inhibitor, the beta-adrenergic receptor inhibitor, and the G-CSF induces an enhanced amount of cell mobilization relative to the amount of cell mobilization induced by the CXCR4 inhibitor and the beta-adrenergic receptor inhibitor only. In embodiments, the administering a combination of the CXCR4 inhibitor, the beta-adrenergic receptor inhibitor, and the G-CSF mobilizes a cell by an amount enhanced relative to the amount of cell mobilization induced by the CXCR4 inhibitor and the beta-adrenergic receptor inhibitor only. In embodiments, the administering a combination of TG-0054 (burixafor) and the G-CSF induces an enhanced amount of cell mobilization relative to the amount of cell mobilization induced by AMD3100 (plerixafor) and the G-CSF. In embodiments, the administering a combination of the TG-0054 (burixafor) and the G-CSF mobilizes a cell by an amount enhanced relative to the amount of cell mobilization induced by the AMD3100 (plerixafor) and the G-CSF. In some embodiments, the enhanced amount of cell mobilization relative to the amount of cell mobilization induced by the CXCR4 inhibitor only is between 1.1-fold to 1.2-fold, 1.2-fold to 1.3-fold, 1.3-fold to 1.4-fold, 1.4-fold to 1.5-fold, 1.5-fold to 1.6-fold, 1.6-fold to 1.7-fold, 1.7-fold to 1.8-fold, 1.8-fold to 1.9-fold, 1.9-fold to 2-fold, 2-fold to 2.5-fold, 2.5-fold to 3-fold, 3-fold to 4-fold, 4-fold to 5-fold, 5-fold to 10-fold, or 10-fold or more. In some embodiments, the enhanced amount of cell mobilization relative to the amount of cell mobilization induced by the CXCR4 inhibitor only is between 5%-10% more, 10%-20% more, 20%-30% more, 30%-40% more, 40%-50% more, 50%-60% more, 60%-70% more, 70%-80% more, 80%-90% more, 90%-100% more, 100%-120% more, 120%-140% more, 140%-160% more, 160%-180% more, 180%-200% more, 200%-250% more, 250%-300% more, 300%-400% more, 400%-500% more, 500%-750% more, 750%-1000% more, or 1000% or more. In embodiments, an enhanced amount of cell mobilization or apheresis is measured by a method selected from the group consisting of complete blood count (CBC) analysis, flow cytometry, and colony forming unit (CFU) assay. In embodiments, the enhanced amount of cell mobilization or apheresis is measured by flow cytometry. In embodiments, the flow cytometry is performed on (Lin−Sca1+c−Kit+) LSK cells. In embodiments, the enhanced amount of cell mobilization or apheresis is measured by colony forming unit (CFU) assay.

In embodiments, the subject has a CXCR4 protomer in the cell. In embodiments, the subject has an ADRB2 protomer in the cell. In embodiments, the subject has a CXCR4 protomer and an ADRB2 protomer in the cell. In embodiments, the subject has a CXCR4-ADRB2 heteromer in the cell. In embodiments, i) the CXCR4-ADRB2 heteromer has an enhanced amount of downstream calcium mobilization relative to downstream calcium mobilization from a CXCR4 protomer or ADRB2 protomer; and ii) the administered combination of inhibitors suppresses the enhanced downstream calcium mobilization from said CXCR4-ADRB2 heteromer in the stem cell.

In embodiments, the cell is a stem cell. In embodiments, the stem cell is selected from the group consisting of a hematopoietic stem cell, a hematopoietic progenitor cell, a mesenchymal stem cell, an endothelial progenitor cell, a neural stem cell, an epithelial stem cell, a skin stem cell, and a cancer stem cell. In embodiments, the stem cell is a hematopoietic stem cell or a hematopoietic progenitor cell. In embodiments, the hematopoietic stem cell or the hematopoietic progenitor cell is mobilized from bone marrow to peripheral blood. In embodiments, the mobilized hematopoietic stem cell or hematopoietic progenitor cell is collected for transplantation to a patient having cancer. In embodiments, the cancer is selected from the group consisting of lymphoma, leukemia, and myeloma. In embodiments, the cancer is non-Hodgkin lymphoma (NHL), acute myeloid leukemia (AML), acute lymphoblastic leukemia (ALL), or multiple myeloma (MM). In embodiments, the stem cell is a mesenchymal stem cell. In embodiments, the mesenchymal stem cell is mobilized from bone marrow to peripheral blood. In embodiments, the mesenchymal stem cell is mobilized for treatment of a condition selected from the group consisting of neurological disorder, cardiac ischemia, myocardial infarction, diabetes, tissue repair, bone and cartilage disease, autoimmune disease, graft versus host disease, Crohn's disease, multiple sclerosis, systemic lupus erythematosus, and systemic sclerosis. In embodiments, the stem cell is a cancer stem cell. In embodiments, the cancer stem cell is mobilized into blood. In embodiments, the cancer stem cell is mobilized for treatment of a cancer.

In embodiments, the cell is an immune cell. In embodiments, the immune cell is a white blood cell. In embodiments, the white blood cell is a lymphocyte. In embodiments, the lymphocyte is selected from the group consisting of a T cell, a B cell, and a natural killer (NK) cell. In embodiments, the lymphocyte is a T cell. In embodiments, the lymphocyte is a natural killer (NK) cell. In embodiments, the white blood cell is a granulocyte. In embodiments, the granulocyte is selected from the group consisting of a neutrophile, an eosinophile, and a basophile. In embodiments, the granulocyte is a neutrophile. In embodiments, the white blood cell is a monocyte. In embodiments, the immune cell is mobilized from bone marrow to peripheral blood. In embodiments, the immune cell is mobilized from lymph node to peripheral blood. In embodiments, the mobilized immune cell is used for adoptive cell therapy (ACT). In embodiments, the adoptive cell therapy (ACT) is chimeric antigen receptor (CAR) T cell therapy. In embodiments, the adoptive cell therapy (ACT) is natural killer (NK) cell therapy. In embodiments, the adoptive cell therapy (ACT) is engineered T-cell receptor (TCR) therapy. In embodiments, the adoptive cell therapy (ACT) is tumor-infiltrating lymphocyte (TIL) therapy.

In some embodiments of the present invention, the mobilizing a cell in a subject comprises blocking CXCR4. Many antiviral agents that inhibit HIV replication via inhibition of CXCR4, the co-receptor required for fusion and entry of T-tropic HIV strains, also inhibit the binding and signaling induced by the natural ligand, the chemokine SDF-1 (also known as CXCL12). While not wishing to be bound by any theory, the agents which inhibit the binding of SDF-1 to CXCR4 can effect an increase in mobilization of stem and/or progenitor cells to the periphery by virtue of such inhibition. Enhancing mobilization of the stem and/or progenitor cells to peripheral blood is helpful in treatments to alleviate the effects of protocols that adversely affect the bone marrow, such as those that result in leukopenia, which are known side effects of chemotherapy and radiotherapy. The agents inhibiting the binding of SDF-1 to CXCR4 also enhance the success of bone marrow transplantation, enhance wound healing and burn treatment, and aid in restoration of damaged organ tissue. They also combat bacterial infections that are prevalent in leukemia. They are used to mobilize and harvest CD34+ cells via apheresis with and without combinations with other mobilizing factors. The harvested cells are used in treatments requiring stem cell transplantations.

In some embodiments of the present invention, mobilizing a stem cell in a subject comprises blocking a CXCR4-GPCR heteromer. Various CXCR4-GPCR heteromers with distinct physiological and pharmacological properties have been reported, but their roles in stem cell mobilization or possibilities for developing stem cell mobilization therapeutics targeting CXCR4-GPCR heteromers have not been clearly understood or appreciated.

In the art, GPCRs were believed to function as monomers that interact with hetero-trimeric G proteins upon ligand binding, and drugs were developed based on monomeric or homomeric GPCRs (Milligan 2008). Recently, this view changed drastically based on discoveries that GPCRs can form heteromers, and that heteromerization is obligatory for some GPCRs. GPCR heteromerization is known to alter GPCR maturation and cell surface delivery, ligand binding affinity, signaling intensity and pathways, as well as receptor desensitization and recycling (Terrillon and Bouvier 2004; Ferre et al., 2010; Rozenfeld and Devi 2010; Gomes et al., 2016; Farran 2017). Different GPCR heteromers display distinct functional and pharmacological properties, and GPCR heteromerization can vary depending on cell types, tissues, and diseases or pathological conditions (Terrillon and Bouvier 2004; Ferre et al., 2010; Rozenfeld and Devi 2010; Gomes et al., 2016; Farran 2017). GPCR heteromerization is currently regarded as a general phenomenon, and deciphering GPCR heteromerization opens new avenues for understanding receptor function, physiology, roles in diseases and pathological conditions. Accordingly, identification of GPCR heteromers and their functional properties offers new opportunity for developing new pharmaceuticals or finding new use of old drugs with fewer side effects, higher efficacy, and increased tissue selectivity (Ferre et al., 2010; Rozenfeld and Devi 2010; Farran 2017).

Apheresis is a standard practice to obtain a larger number of immune cells as starting material for Adoptive Cell Therapy (ACT), which is a treatment based on transferring cells into a patient (1-3). Apheresis may involve passing the blood of a patient through an apparatus that separates out one particular constituent and returns the remainder to the blood circulation of the patient. Apheresis is thus an extracorporeal therapy. Depending on the substance being removed, different processes are employed in apheresis. If separation by density is required, centrifugation is the most common method. Other methods involve absorption onto beads coated with an absorbent material and filtration. The centrifugation method can be divided into two basic categories: continuous flow centrifugation (CFC) and intermittent flow centrifugation.

CFC historically required two venipunctures as the “continuous” means that the blood was collected, spun, and returned simultaneously. Newer systems can use a single venipuncture. The main advantage of CFC is the low extracorporeal volume (calculated by volume of the apheresis chamber, the donor's hematocrit, and total blood volume of the donor) used in the procedure, which may be advantageous in the elderly and for children. Intermittent flow centrifugation works in cycles, taking blood, spinning/processing the blood, then giving back the unused parts to the donor in a bolus. The main advantage is a single venipuncture site. To stop the blood from coagulating, anticoagulant is automatically mixed with the blood as it is pumped from the body into the apheresis machine.

The various apheresis techniques may be used whenever the removed constituent is causing severe symptoms of disease in a patient. Generally, apheresis has to be performed fairly often and is an invasive procedure. It is therefore generally employed if other means to control a particular disease have failed, or if the symptoms are of such a nature that waiting for medication to become effective would cause suffering or risk of complications. Apheresis techniques include: (1) plasma exchange-removal of the liquid portion of blood to remove harmful substances, where the plasma is replaced with a replacement solution; (2) LDL apheresis—removal of low density lipoprotein in patients with familial hypercholesterolemia; (3) photopheresis—used to treat graft-versus-host disease, cutaneous T-cell lymphoma, and rejection in heart transplantation; (4) immunoadsorbtion with Staphylococcal protein A-agarose column—removal of allo- and autoantibodies (in autoimmune diseases, transplant rejection, hemophilia) by directing plasma through protein A-agarose columns (Protein A is a cell wall component produced by several strains of Staphylococcus aureus which binds to the Fc region of IgG); (5) leukocytapheresis-removal of malignant white blood cells in people with leukemia and very high white blood cell counts causing symptoms; (6) erythrocytapheresis—removal of erythrocytes (red blood cells) in people with iron overload as a result of Hereditary haemochromatosis or transfusional iron overload; (7) thrombocytapheresis—removal of platelets in people with symptoms from extreme elevations in platelet count such as those with essential thrombocythemia or polycythemia vera; and (8) leukapheresis-separates out excess white blood cells of leukemia patients while recycling the remainder of their blood.

Apheresis is a difficult procedure, inconvenient and expensive. With the rapid growth of ACTs including CAR-T, CAR-NK, Tumor-Infiltrating Lymphocyte (TIL), and engineered T cell receptor (TCR), the need for apheresis technology for the routine production of pure immune cells is increasing (2). The industry that supplies GMP-grade starting materials for ACTs is also growing rapidly (4-5). Thus, stem cell mobilization technologies that can control types of immune cells and improve the yield of apheresis have become important.

Enhanced stem cell mobilization (SCM) or cell mobilization methods as disclosed herein, can further augment or facilitate the conventional apheresis procedure. In a specific embodiment, enhanced stem cell mobilization (SCM) or cell mobilization is particularly beneficial for the apheresis technique of leukapheresis. In some embodiments, administering a CXCR4 antagonist to a subject further enhances apheresis by augmenting SCM or cell mobilization. In some embodiments, administering a beta-adrenergic receptor antagonist in conjunction with a CXCR4 antagonist to a subject further enhances apheresis by augmenting SCM or cell mobilization, and/or replacing the G-CSF component of the treatment regime with a non-selective beta-blocker, such as propranolol. In some embodiments, the augmentation of SCM in turn benefits HSCT (Hematopoietic Stem Cells Transplantation) or manufacturing of CAR-T cells for cancer immunotherapy. Currently, CXCR4 inhibitors, such as plerixafor (Mozobil) which have been approved as stem cell mobilizers, are being used together with G-CSF as the standard of care to provide enriched hematopoietic stem cells and progenitor cells from healthy donors, marketed as the product “mobilized leukopaks.”

Disclosed herein are methods of enhancing apheresis in a subject, the method comprising: blocking CXCR4 signaling and beta-adrenergic receptor signaling in the subject. Also disclosed herein are methods of enhancing apheresis by inducing cell mobilization in a subject, the method comprising: blocking CXCR4 signaling and beta-adrenergic receptor signaling in the subject. Further disclosed herein are methods of enhancing apheresis by mobilizing a cell in a subject, the method comprising: blocking CXCR4 signaling and beta-adrenergic receptor signaling in the subject. In embodiments, the blocking beta-adrenergic receptor signaling is performed before the blocking CXCR4 signaling. In some embodiments, the blocking beta-adrenergic receptor signaling is performed at a first specific time interval before the blocking CXCR4 signaling. In some embodiments, the first specific time interval is between 5 minutes to 10 minutes, 10 minutes to 20 minutes, 20 minutes to 30 minutes, 30 minutes to 40 minutes, 40 minutes to 50 minutes, 50 minutes to 1 hour, 1 hour to 2 hours, 2 hours to 3 hours, 3 hours to 4 hours, 4 hours to 5 hours, 5 hours to 6 hours, 6 hours to 12 hours, 12 hours to 24 hours, 1 day to 2 days, 2 days to 3 days, 3 days to 4 days, 4 days to 5 days, 5 days to 6 days, 6 days to 7 days, 7 days to 8 days, 8 days to 9 days, 9 days to 10 days, 10 days to 11 days, 11 days to 12 days, 12 days to 13 days, 13 days to 14 days, or 14 days or more. In embodiments, the blocking beta-adrenergic receptor signaling continues after the blocking CXCR4 signaling is terminated. In some embodiments, the blocking beta-adrenergic receptor signaling continues for a second specific time interval after the blocking CXCR4 signaling is terminated. In some embodiments, the second specific time interval is between 5 minutes to 10 minutes, 10 minutes to 20 minutes, 20 minutes to 30 minutes, 30 minutes to 40 minutes, 40 minutes to 50 minutes, 50 minutes to 1 hour, 1 hour to 2 hours, 2 hours to 3 hours, 3 hours to 4 hours, 4 hours to 5 hours, 5 hours to 6 hours, 6 hours to 12 hours, 12 hours to 24 hours, 1 day to 2 days, 2 days to 3 days, 3 days to 4 days, 4 days to 5 days, 5 days to 6 days, 6 days to 7 days, 7 days to 8 days, 8 days to 9 days, 9 days to 10 days, 10 days to 11 days, 11 days to 12 days, 12 days to 13 days, 13 days to 14 days, or 14 days or more.

In embodiments, the blocking CXCR4 signaling comprises administering a CXCR4 inhibitor to the subject.

Disclosed herein are methods of enhancing apheresis in a subject, the method comprising: administering a beta-adrenergic receptor inhibitor and a CXCR4 inhibitor to the subject. Also disclosed herein are methods of enhancing apheresis by inducing cell mobilization in a subject, the method comprising: administering a beta-adrenergic receptor inhibitor and a CXCR4 inhibitor to the subject. Further disclosed herein are methods of enhancing apheresis by mobilizing a cell in a subject, the method comprising: administering a beta-adrenergic receptor inhibitor and a CXCR4 inhibitor to the subject. In some embodiments, the administering the beta-adrenergic receptor inhibitor is performed at a first specific time interval before the administering the CXCR4 inhibitor. In some embodiments, the first specific time interval is between 5 minutes to 10 minutes, 10 minutes to 20 minutes, 20 minutes to 30 minutes, 30 minutes to 40 minutes, 40 minutes to 50 minutes, 50 minutes to 1 hour, 1 hour to 2 hours, 2 hours to 3 hours, 3 hours to 4 hours, 4 hours to 5 hours, 5 hours to 6 hours, 6 hours to 12 hours, 12 hours to 24 hours, 1 day to 2 days, 2 days to 3 days, 3 days to 4 days, 4 days to 5 days, 5 days to 6 days, 6 days to 7 days, 7 days to 8 days, 8 days to 9 days, 9 days to 10 days, 10 days to 11 days, 11 days to 12 days, 12 days to 13 days, 13 days to 14 days, or 14 days or more. In embodiments, the administering the beta-adrenergic receptor inhibitor continues after the administering the CXCR4 inhibitor is terminated. In some embodiments, the administering the beta-adrenergic receptor inhibitor continues for a second specific time interval after the administering the CXCR4 inhibitor is terminated. In some embodiments, the second specific time interval is between 5 minutes to 10 minutes, 10 minutes to 20 minutes, 20 minutes to 30 minutes, 30 minutes to 40 minutes, 40 minutes to 50 minutes, 50 minutes to 1 hour, 1 hour to 2 hours, 2 hours to 3 hours, 3 hours to 4 hours, 4 hours to 5 hours, 5 hours to 6 hours, 6 hours to 12 hours, 12 hours to 24 hours, 1 day to 2 days, 2 days to 3 days, 3 days to 4 days, 4 days to 5 days, 5 days to 6 days, 6 days to 7 days, 7 days to 8 days, 8 days to 9 days, 9 days to 10 days, 10 days to 11 days, 11 days to 12 days, 12 days to 13 days, 13 days to 14 days, or 14 days or more.

In embodiments, the beta-adrenergic receptor inhibitor is an ADRB2 inhibitor. In embodiments, the beta-adrenergic receptor inhibitor is selected from the group consisting of alprenolol, atenolol, betaxolol, bupranolol, butoxamine, carazolol, carvedilol, CGP 12177, cicloprolol, ICI 118551, ICYP, labetalol, levobetaxolol, levobunolol, LK 204-545, metoprolol, nadolol, NIHP, NIP, propafenone, propranolol, sotalol, SR59230A, and timolol. In embodiments, the beta-adrenergic receptor inhibitor is selected from the group consisting of propranolol, nadolol, and ICI 118551. In embodiments, the beta-adrenergic receptor inhibitor is propranolol.

In embodiments, the CXCR4 inhibitor is selected from the group consisting of ALX40-4C, AMD070 (AMD11070, X4P-001), AMD3100 (plerixafor), AMD3465, ATI 2341, BKT140 (BL-8040; TF14016; 4F-Benzoyl-TN14003), CTCE-9908, CX549, D-[Lys3] GHRP-6, FC122, FC131, GMI-1359, GSK812397, GST-NT21MP, isothiourea-la, isothiourea-1t (IT1t), KRH-1636, KRH-3955, LY2510924, MSX-122, N-[11C] Methyl-AMD3465, POL6326, SDF-1 1-9 [P2G] dimer, SDF1 P2G, T134, T140, T22, TC 14012, TG-0054 (Burixafor), USL311, viral macrophage inflammatory protein-II (vMIP-II), WZ811, [64Cu]-AMD3100, [64Cu]-AMD3465, [68Ga] pentixafor, [90Y] pentixather, [99mTc] 02-AMD3100, [177Lu] pentixather, and 508MC1 (Compound 26). In embodiments, the CXCR4 inhibitor is selected from the group consisting of AD-214, AMD070 (AMD11070, X4P-001), AMD3100 (plerixafor), BKT140 (BL-8040; TF14016; 4F-Benzoyl-TN14003), CTCE-9908, LY2510924, LY2624587, T140, TG-0054 (Burixafor), PF-06747143, POL6326, and ulocuplumab (MDX1338/BMS-936564). In embodiments, the CXCR4 inhibitor is TG-0054 (burixafor). In embodiments, the CXCR4 inhibitor is AMD3100 (plerixafor). In embodiments, the CXCR4 inhibitor is ulocuplumab (MDX1338/BMS-936564).

In embodiments, the administering the CXCR4 inhibitor to the subject comprises administering TG-0054 (burixafor) and propranolol. In embodiments, the administering the CXCR4 inhibitor to the subject comprises administering AMD3100 (plerixafor) and propranolol. In embodiments, the administering the CXCR4 inhibitor to the subject comprises administering ulocuplumab (MDX1338/BMS-936564) and propranolol.

In embodiments, the method further comprises administering G-CSF to the subject. In embodiments, the administering the beta-adrenergic receptor inhibitor and the CXCR4 inhibitor to the subject is performed in the absence of G-CSF. Disclosed herein are methods of enhancing apheresis in a subject, the method comprising: administering a CXCR4 inhibitor and G-CSF to the subject, in the absence of a beta-adrenergic receptor inhibitor. Further disclosed herein are methods of enhancing apheresis by inducing cell mobilization in a subject, the method comprising: administering a CXCR4 inhibitor and G-CSF to the subject, in the absence of a beta-adrenergic receptor inhibitor. Also disclosed herein are methods of enhancing apheresis by mobilizing a cell in a subject, the method comprising: administering a CXCR4 inhibitor and G-CSF to the subject, in the absence of a beta-adrenergic receptor inhibitor. In embodiments, the administering a combination of the CXCR4 inhibitor and the G-CSF induces an enhanced amount of apheresis relative to the amount of apheresis induced by the CXCR4 inhibitor only. In embodiments, the administering a combination of the CXCR4 inhibitor and the beta-adrenergic receptor inhibitor induces an enhanced amount of apheresis relative to the amount of apheresis induced by the CXCR4 inhibitor only. In embodiments, the administering a combination of the CXCR4 inhibitor and the beta-adrenergic receptor inhibitor, and the G-CSF induces an enhanced amount of apheresis relative to the amount of apheresis induced by the CXCR4 inhibitor and the beta-adrenergic receptor inhibitor only. In embodiments, the administering a combination of the TG-0054 (burixafor) and the G-CSF induces an enhanced amount of apheresis relative to the amount of apheresis induced by the AMD3100 (plerixafor) and the G-CSF. In some embodiments, the enhanced amount of cell mobilization relative to the amount of cell mobilization induced by the CXCR4 inhibitor only is between 1.1-fold to 1.2-fold, 1.2-fold to 1.3-fold, 1.3-fold to 1.4-fold, 1.4-fold to 1.5-fold, 1.5-fold to 1.6-fold, 1.6-fold to 1.7-fold, 1.7-fold to 1.8-fold, 1.8-fold to 1.9-fold, 1.9-fold to 2-fold, 2-fold to 2.5-fold, 2.5-fold to 3-fold, 3-fold to 4-fold, 4-fold to 5-fold, 5-fold to 10-fold, or 10-fold or more. In some embodiments, the enhanced amount of cell mobilization relative to the amount of cell mobilization induced by the CXCR4 inhibitor only is between 5%-10% more, 10%-20% more, 20%-30% more, 30%-40% more, 40%-50% more, 50%-60% more, 60%-70% more, 70%-80% more, 80%-90% more, 90%-100% more, 100%-120% more, 120%-140% more, 140%-160% more, 160%-180% more, 180%-200% more, 200%-250% more, 250%-300% more, 300%-400% more, 400%-500% more, 500%-750% more, 750%-1000% more, or 1000% or more. In embodiments, an enhanced amount of cell mobilization or apheresis is measured by a method selected from the group consisting of complete blood count (CBC) analysis, flow cytometry, and colony forming unit (CFU) assay. In embodiments, the enhanced amount of cell mobilization or apheresis is measured by flow cytometry. In embodiments, the flow cytometry is performed on (Lin−Sca1+c−Kit+) LSK cells. In embodiments, the enhanced amount of cell mobilization or apheresis is measured by colony forming unit (CFU) assay.

Further information regarding the ADRB2, evaluated herein as forming heteromers with CXCR4, are detailed below:

ADRB2—The beta-2 adrenergic receptor (B2 adrenoreceptor), also known as ADRB2, is a cell membrane-spanning beta-adrenergic receptor that interacts with epinephrine, a hormone and neurotransmitter (ligand synonym, adrenaline) whose signaling, via a downstream L-type calcium channel interaction, mediates physiologic responses such as smooth muscle relaxation and bronchodilation (Gregorio et al., 2017). ADRB2 functions in muscular system such as smooth muscle relaxation, motor nerve terminals, glycogenolysis and in circulatory system such as heart muscle contraction, cardiac output increase. In the normal eye, beta-2 stimulation by salbutamol increases intraocular pressure via net. In digestive system, the ADRB2 induces glycogenolysis and gluconeogenesis in liver and insulin secretion from pancreas (Fitzpatrick, 2004).

ADRB2 signaling in the cardiac myocyte is modulated by interactions with CXCR4 (LaRocca et al., 2010). Norepinephrine attenuates CXCR4 expression and the corresponding invasion of MDA-MB-231 breast cancer cells via ADRB2 (Wang et al., 2015a). ADRB2 is expressed in several cancers such as pancreatic, prostate (Braadland et al., 2014; Xu et al., 2017), renal and breast cancer (Choy et al., 2016).

Alternative methods for detecting heteromer formation include, but are not limited to: immunostaining (Bushlin et al., 2012; Decaillot et al., 2008); immunoelectron microscopy (Fernandez-Duenas et al., 2015); BRET (Pfleger and Eidne, 2006); Time-resolved FRET assays (Fernandez-Duenas et al., 2015); In Situ Hybridization (He et al., 2011); FRET (Lohse et al., 2012); β-arrestin recruitment assay using GPCR heteromer identification technology (GPCR-HIT, Dimerix Bioscience) (Mustafa and Pfleger, 2011) using BRET, FRET, BiFC, Bimolecular Luminescence Complementation, enzyme fragmentation assay, and Tango Tango GPCR assay system (Thermo Fisher Scientific) (Mustafa, 2010); PRESTO-Tango system (Kroeze et al., 2015); regulated secretion/aggregation technology (ARIAD Pharmaceuticals) (Hansen et al., 2009); Receptor Selection and Amplification Technology (ACADIA Pharmaceuticals) (Hansen et al., 2009); DimerScreen (Cara Therapeutics) (Mustafa, 2010); Dimer/interacting protein translocation assay (Patobios) (Mustafa, 2010); Co-immunoprecipitation (Abd Alla et al., 2009); GPCR internalization assays using surface enzyme-linked immunosorbent assay (ELISA) (Decaillot et al., 2008) or Flow Cytometry (Law et al., 2005); Whole Cell Phosphorylation Assays (Pfeiffer et al., 2002); and Proximity-ligation assay (PLA) (Frederick et al., 2015).

Alternative methods for detecting changes in pharmacological properties, signaling properties, and/or trafficking properties, in cells expressing both CXCR4 and GPCRx include, but are not limited to: Radioligand Binding Assays (Bushlin et al., 2012; Pfeiffer et al., 2002); Cell Surface Biotinylation and Immunoblotting (He et al., 2011); immunostaining (Bushlin et al., 2012; Decaillot et al., 2008); immunoelectron microscopy (Fernandez-Duenas et al., 2015); [35S]GTPγS Binding assays (Bushlin et al., 2012); Calcium imaging or assays using dyes such as Fura 2-acetomethoxy ester (Molecular Probes), Fluo-4 NW calcium dye (Thermo Fisher Scientific), or FLIPR5 dye (Molecular Devices); cAMP assays using radioimmunoassay kit (Amersham Biosciences); AlphaScreen (PerkinElmer Life Sciences); Parameter Cyclic AMP Assay (R&D Systems); femto CAMP kit (Cisbio); cAMP Direct Immunoassay Kit (Calbiochem) or GloSensor CAMP assay (Promega); GTPase assay (Pello et al., 2008); PKA activation (Stefan et al., 2007); ERK1/2 and/or Akt/PKB Phosphorylation Assays (Callen et al., 2012); Src and STAT3 phosphorylation assays (Rios et al., 2006); reporter assays such as cAMP response element (CRE); nuclear factor of activated T-cells response element (NFAT-RE); serum response element (SRE); serum response factor response element (SRF-RE); and NF-κB-response element luciferase reporter assays; Secreted alkaline phosphatase Assay (Decaillot et al., 2011); Measurement of Inositol 1-Phosphate Production Using TR-FRET or [3H]myo-Inositol (Mustafa et al., 2012); RT-qPCR for measuring downstream target gene expression (Mustafa et al., 2012); and Adenylyl Cyclase Activity (George et al., 2000); next generation sequencing (NGS); and any other assay that can detect a change in receptor function as a result of receptor heterodimerization.

The phrase “protein-protein interaction inhibitor,” “PPI inhibitor,” or their variants as used herein refer to any molecules that can interfere with protein-protein interactions. Protein-protein interaction, unlike enzyme-substrate interaction involving well-defined binding pockets, is a transient interaction or association between proteins over relatively large areas and is often driven by electrostatic interactions, hydrophobic interactions, hydrogen bonds, and/or Van der Waals forces. PPI inhibitors may include, but not limited to, membrane-permeable peptides or lipid fused to a peptide sequence that disrupts the GPCR heteromeric interface, for example, transmembrane helix, intracellular loop, or C-terminal tail of GPCRx. The PPI inhibitor of the CXCR4-GPCRx heteromer, for example, may be a membrane-permeable peptide or cell-penetrating peptide (CPP) conjugated with peptide that targets the CXCR4-GPCRx heteromeric interface(s), or may be a cell-penetrating lipidated peptide targeting the CXCR4-GPCRx heteromeric interface(s).

For example, the membrane-permeable peptide or cell-penetrating peptide includes: HIV-1 TAT peptides, such as TAT48-60 and TAT49-57; Penetratins, such as pAntp (43-58); Polyarginines (Rn such as R5 to R12); Diatos peptide vector 1047 (DPV1047, Vectocell®); MPG (HIV gp41 fused to the nuclear localization signal (NLS) of the SV40 large T antigen); Pep-1 (tryptophan-rich cluster fused to the NLS of SV40 large T antigen); pVEC peptide (vascular endothelial cadherin); p14 alternative reading frame (ARF) protein-based ARF (1-22); N-terminus of the unprocessed bovine prion protein BPrPr (1-28); Model amphipathic peptide (MAP); Transportans; Azurin-derived p28 peptide; amphipathic β-sheet peptides, such as VT5; proline-rich CPPs, such as Bac 7 (Bac1-24); hydrophobic CPPs, such as C105Y derived from α1-Antitrypsin; PFVYLI derived from synthetic C105Y; Pep-7 peptide (CHL8 peptide phage clone); and modified hydrophobic CPPs, such as stapled peptides and prenylated peptides (Guidotti et al., 2017; Kristensen et al., 2016). The membrane-permeable peptide or cell-penetrating peptide can further include, for example, TAT-derived cell-penetrating peptides, signal sequence-based (e.g., NLS) cell-penetrating peptides, hydrophobic membrane translocating sequence (MTS) peptides, and arginine-rich molecular transporters. The cell-penetrating lipidated peptide includes, for example, pepducins, such as ICL1/2/3, C-tail-short palmitoylated peptides (Covic et al., 2002; O'Callaghan et al., 2012).

The peptide(s) that target the CXCR4-GPCRx heteromeric interface may be, for example, a transmembrane domain of CXCR4, transmembrane domain of GPCRx, intracellular loop of CXCR4, intracellular loop of GPCRx, C-terminal domain of CXCR4, or C-terminal domain of GPCRx., extracellular loop of CXCR4, extracellular loop of GPCRx, N-terminal region of CXCR4, or N-terminal region of GPCRx.

It is understood that modifications which do not substantially affect the activity of the various embodiments of this invention are also provided within the definition of the invention provided herein. Accordingly, the following examples are intended to illustrate but not limit the invention disclosed herein.

### EXAMPLES

### Example 1. Combination Blockade of Beta 2-Adrenergic Receptor and CXCR4 Signaling in Stem Cell Mobilization: Preclinical Evidence

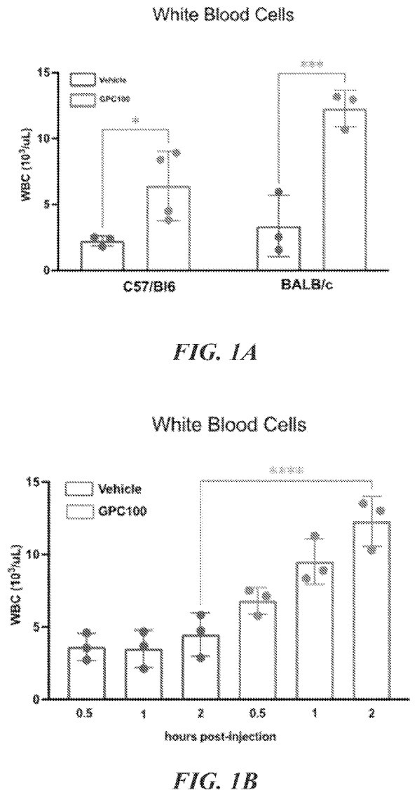

To identify novel CXCR4-GPCRx heteromers, recombinant adenoviruses encoding 143 GPCRs fused with N-terminal fragments of yellow fluorescent protein Venus (VN) and 147 GPCRs fused with C-terminal fragment of Venus (VC) were made as described in Song et al. (Song et al., 2014; SNU patent; Song, thesis). CXCR4-GPCR heteromers were identified using bimolecular fluorescence complementation (BiFC) assay (FIG. 1), in which two complementary VN and VC fragments of Venus reconstitute a fluorescent signal only when both fragments are close enough through interaction between two different proteins to which they are fused (Hu et al., 2002).

The preclinical study evaluated the ability of the non-selective beta adrenergic receptor blocker propranolol to improve GPC100-induced stem cell mobilization following a seven-day treatment in a mouse model. These effects were further assessed by the addition of G-CSF to GPC100, as well as in comparison with the current standard of care treatments for stem cell mobilization such as G-CSF alone or in combination with AMD3100.

### Materials & Methods

### Compounds

Propranolol (MedChem Express, Princeton, NJ) was intraperitoneally (IP) administered at 20 mg/kg for seven days once in a day. Recombinant murine G-CSF (Peprotech, Cranbury, NJ) was administered subcutaneously (SC) two times a day for five days at 0.1 mg/kg/dose. AMD3100 (MedChem Express, Princeton, NJ) was administered subcutaneously once on day 7 at 5 mg/kg. GPC100 was administered intravenously (IV) once on day 7 at 30 mg/kg. GPC100 was acquired by GPCR Therapeutics from TaiGen Biotechnology, Taiwan. All compounds were reconstituted in PBS. Vehicle controls received PBS intravenously, intraperitoneally or subcutaneously depending on the drug combination used in the study.

### Mice

C57BL/6 and BALB/c mice (female, 6-9 weeks old) were purchased from Jackson Laboratory and maintained on a 12-h light/dark cycle with free access to food and water. All mice were housed at the laboratory animal facility that had been accredited by AAALAC (Association for Assessment and Accreditation of Laboratory Animal Care International) and the IACUC (Institutional Animal Care and Use Committee) of Crown Bioscience (San Diego,CA) or Explora Biolabs (San Carlos, CA).

### Experimental Design

For the pilot study, C57BL/6 and BALB/c mice were administered a single dose of GPC100 (30 mg/kg, IV) or vehicle (IV) and blood was collected one hour later. Another group of C57/BL6 mice received a single dose of GPC100 (30 mg/kg, IV) and blood was collected at 30 min, 1 hour and 2 hours post-injection. The time point for sample collection post-GPC100 was established at 2 hours based on the maximum WBC mobilization (FIG. 1B). All subsequent studies were performed in C57/BL6 female mice as this mouse strain is more critically evaluated in stem cell mobilization studies.

To determine the effects of propranolol on GPC100-induced mobilization, mice received vehicle (IP) or propranolol (20 mg/kg, IP) for seven days. On day 7, GPC100 (30 mg/kg, IV) was co-administered (Table 3). To determine if propranolol alone alters the blood cell count, it was administered for seven days, followed by an intravenous vehicle injection on day 7. Mice were treated with propranolol or vehicle for seven days; GPC100 or vehicle was co-adminstered on day 7 to determine the effects of propranolol alone, GPC100 alone or their combination on total blood cell count in peripheral blood (Table 3).

TABLE 3
Dosing regimen for propranolol and
GPC100 combination treatment.

| | Day | Day | Day | Day | Day | Day | Day |
|---|---|---|---|---|---|---|---|
| Drug | 1 | 2 | 3 | 4 | 5 | 6 | 7 |
| Propranolol | + | + | + | + | + | + | + |
| 20 mg/kg IP | | | | | | | |
| Or vehicle IP | | | | | | | |
| GPC100 | | | | | | | + |
| 30 mg/kg IV | | | | | | | |
| Or vehicle IV | | | | | | | |

TABLE 4
Dosing regimen for the standard of care treatment

| | Day | Day | Day | Day | Day | Day | Day |
|---|---|---|---|---|---|---|---|
| Drug | 1 | 2 | 3 | 4 | 5 | 6 | 7 |

| | | | | | | |
|---|---|---|---|---|---|---|
| G-CSF BID | + | + | + | + | + | |
| 100 ug/kg/ | | | | | | |
| injection SC | | | | | | |
| AMD 3100 | | | | | | + |
| 5 mg/kg SC | | | | | | |

TABLE 5
Dosing regimen for the combination
of propranolol and G-CSF + AMD3100

| | Day | Day | Day | Day | Day | Day | Day |
|---|---|---|---|---|---|---|---|
| Drug | 1 | 2 | 3 | 4 | 5 | 6 | 7 |
| Propranolol | + | + | + | + | + | + | + |
| 20 mg/kg IP | | | | | | | |
| G-CSF BID | | + | + | + | + | + | |
| 100 ug/kg/ | | | | | | | |
| injection SC | | | | | | | |
| AMD 3100 | | | | | | | + |
| 5 mg/kg SC | | | | | | | |

In another study, mice were administered G-CSF (0.1 mg/kg, SC, BID) for five days (day 2 to day 6) with or without propranolol. GPC100 (30 mg/kg, IV) was co-administered with propranolol or alone on day 7 (Table 5).

The effect of propranolol on GPC100-induced mobilization was compared with the current standard of care treatment for stem cell mobilization namely G-CSF with or without AMD3100. In this study, G-CSF (0.1 mg/kg, SC, BID) was administered for five days followed by a single injection of a vehicle (SC) or AMD3100 (5 mg/kg, SC) on the following day (Table 4).

Blood was collected by terminal cardiac puncture 2 hours post-GPC100 and 1 hour post-AMD3100 administration. Complete blood counts were obtained by Abaxis hematology analyzer (Abaxis, Union City, CA). Circulating WBC count was used as an indicator of stem cell mobilization in all studies.

Effect of propranolol on GPC100-induced mobilization was evaluated in all six studies. In four studies, this effect was compared with the standard of care treatment of G-CSF and AMD3100 combination. G-CSF was added to GPC100 with (triple) or without (double) propranolol in the last three studies. Effects of GPC100, AMD3100 and G-CSF in combination with vehicle were evaluated in one study. No data points were removed unless the sample showed clotting prior to CBC analysis.

Mice treated with vehicle showed mean WBC count of 3.4+/−1.8×10³ cells/uL and lymphocyte count of 2.6+/−1.2×10³ cells/uL of peripheral blood. Vehicle treated mice were included in all studies as a control despite not presented in data graphs.

### Colony Forming Unit (CFU) Assay

In one out of the six studies, mobilization of hematopoietic progenitor cells was evaluated by CFU assay in addition to the WBC count. Mice were dosed at Crown Bioscience (San Diego, CA) and blood in heparinized tubes was shipped to Reach Bio Research (Seattle, CA) overnight at room temperature. Approximately 8×10⁴ cells were incubated in a methylcellulose-based medium with added cytokines that are known to support erythroid and myeloid progenitors. The cultures were incubated in a humidified incubator for approximately 7 days and then colonies were scored by trained personnel.

### Statistical Analyses

Data analyses were performed using Prism (GraphPad) and all data are presented as mean values (mean±SD). Data represented in one figure were generated during the same experiment. Comparisons of data across different dosing conditions were made using repeated measures one-way analysis of variance followed by Tukey's multiple comparison test. Differences between two groups were determined using the Mann-Whitney test. P<0.05 was considered statistically significant for all tests.

### Results

### GPC100 Increased Circulating WBC Count in Mice

A single intravenous administration of GPC100 (30 mg/kg) resulted in the rapid increase of circulating WBCs in C57/BL6 and Balb/c mice, which is reflective of stem cell mobilization (FIG. 1A). In order to determine the time course of GPC-100-induced mobilization, GPC-100 (30 mg/kg) was administered intravenously in naïve C57/B16 mice, and peripheral blood was collected at time points 0.5-, 1- and 2-hours post-injection in different groups. Time dependent increase in WBC count was observed and a 2-hour post-injection sample collection was selected for subsequent studies (FIG. 1B). Furthermore, C57/BL6 mice were selected over Balb/c for future studies as hematopoietic stem cell mobilization has been critically evaluated in this mouse strain (Broxmeyer et al 2005).

GPC100 administration resulted in significantly more WBCs in peripheral blood compared to AMD3100 when administered with or without G-CSF

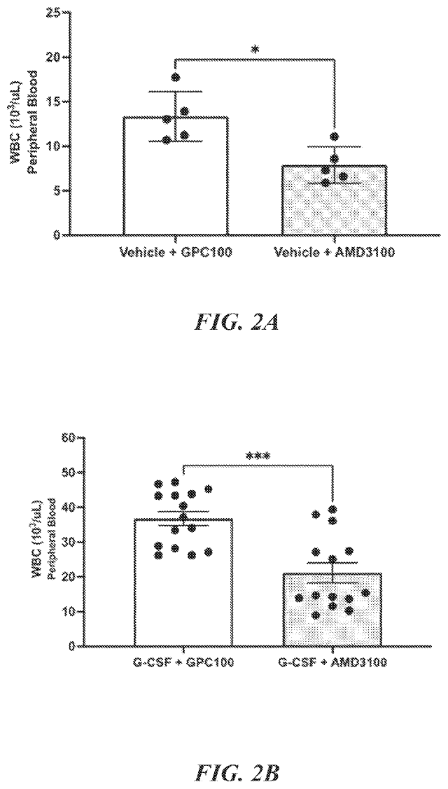

Single injection of GPC100 resulted in a larger increase in circulating WBCs compared to the single injection of AMD3100 following 7-day of vehicle treatment (FIG. 2A). Further increase in WBC count was observed in all the three studies when both GPC100 and AMD3100 were combined with a five-day G-CSF treatment. However, this increase was more significant for GPC100 compared to AMD3100. This supports further evaluation of GPC100 in clinic as a potent stem cell mobilizer (FIG. 2B).

Propranolol enhances GPC100-induced mobilization of WBC into peripheral blood.

Data from six studies indicated that seven-day pretreatment with propranolol significantly increased GPC100-induced WBC count in peripheral blood compared to the seven-day vehicle pretreatment (FIG. 3). When propranolol was administered alone for seven days without GPC100 on day 7, no change in the blood cell count was observed (Table 6). These data indicate that propranolol may improve bone marrow cellularity, thereby enabling GPC100 to mobilize more cells.

TABLE 6
7-day treatment of propranolol alone does not alter the blood cell count

| | WBC | Lymphocytes | Neutrophils | Monocytes | Platelets | RBC | Hemoglobin |
|---|---|---|---|---|---|---|---|
| Treatment | 10³/uL | 10³/uL | 10³/uL | 10³/uL | 10³/uL | 10⁶/uL | g/dL |
| Vehicle | 5.8 ± 0.9 | 4.9 ± 0.7 | 0.7 ± 0.18 | 0.26 ± 0.1 | 309 ± 48 | 9.9 ± 0.32 | 12.3 ± 0.21 |
| Propranolol | 5.4 ± 1.7 | 4.7 ± 1.3 | 0.42 ± 0.3 | 0.2 ± 0.09 | 266 ± 52 | 9.8 ± 0.36 | 13.15 ± 0.8 |

Increased WBC count from the combination treatment of propranolol and GPC100 is comparable to that by G-CSF

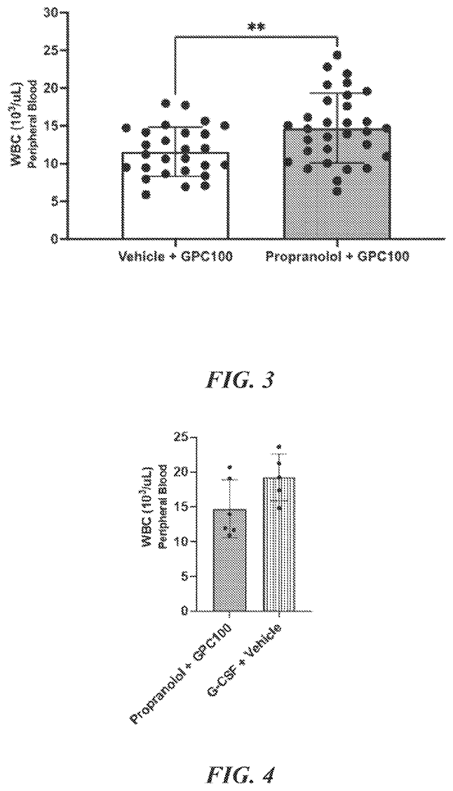

Mice that were administered GPC100 after propranolol pretreatment mobilized white blood cells to the extent that was comparable to those that received G-CSF in 3 out of 6 mice (FIG. 4). Since propranolol can be safely administered orally in patients, its administration may not cause the inconvenience and side effects associated with G-CSF. This warrants more preclinical studies comparing the two groups.

Standard of care treatment of G-CSF and AMD3100 combination mobilizes significantly more WBCs than propranolol and GPC100 combination, but not lymphocytes.

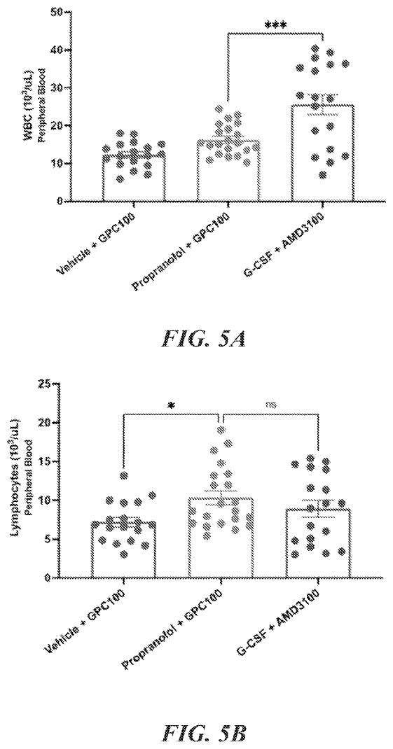

Data collected over four studies showed a large variation in mobilization from the combination treatment of G-CSF and AMD3100 (FIGS. 5A-B). Mobilization induced by the standard of care treatment was significantly greater than GPC100 and propranolol combination treatment for total WBC count. (FIG. 5A). However, comparison of the lymphocyte count showed that the standard of care regimen was comparable to the combination treatment with GPC100 and propranolol (FIG. 5B).

Addition of propranolol to GPC100 and G-CSF resulted in mobilization of more WBC and hematopoietic progenitor cells compared to the standard of care treatment.

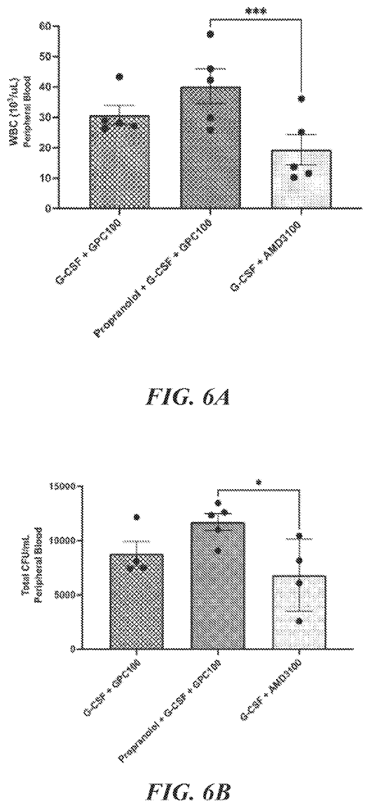

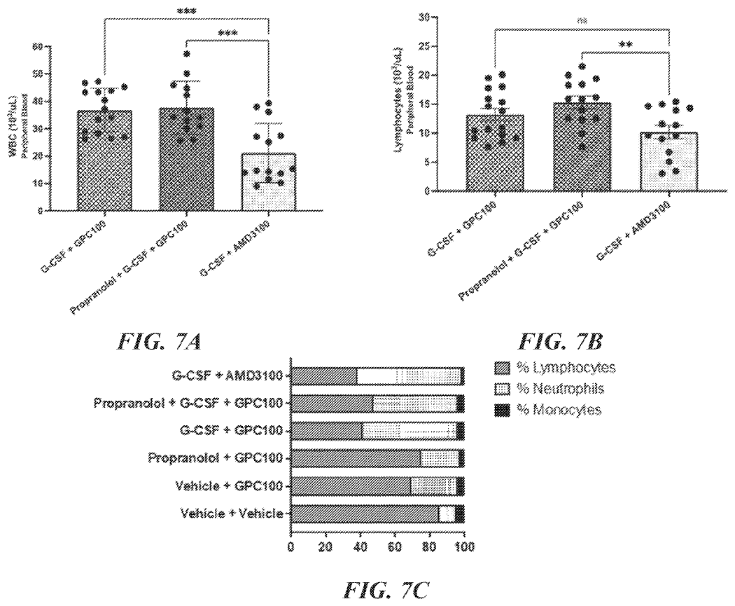

Addition of G-CSF enhanced mobilization by GPC100 with or without propranolol pretreatment. This experiment compared the number of WBC and colony forming units (progenitor cells) in peripheral blood in the same mice. The triple combination which included propranolol, G-CSF and GPC100 was shown to cause maximum mobilization compared to the G-CSF combination treatment in the absence of propranolol for both WBC (FIG. 6A) and progenitor cells (FIG. 6B). When a similar study was repeated, the triple combination was significantly better than the combination treatment of AMD3100 and G-CSF (FIG. 7A).

Propranolol enhances GPC100-induced mobilization of lymphocytes into peripheral blood

Data pooled from all four experiments indicated that when combined with G-CSF, GPC100 mobilized more WBC compared to AMD3100. Addition of propranolol to GPC100 and G-CSF combination treatment mobilized more WBCs including lymphocytes compared to G-CSF and AMD3100 combination (FIGS. 7A-B). Lymphocyte counts were not affected by the addition of propranolol to G-CSF and GPC100 combination (FIG. 7B). Distribution of WBC differential count indicated that addition of propranolol may increase lymphocyte trafficking into the peripheral blood (FIG. 7C). Lymphocyte data for FIG. 7C is presented as % of total white blood cell count (FIG. 7C), where the lymphocyte count for the vehicle/vehicle group was 2.6+/−1.2×10³ cells/uL.)

This provides compelling preclinical evidence to further investigating the effect of propranolol on GPC100-induced lymphocyte mobilization.

### Discussion

The study described herein showed that propranolol increased GPC100-induced mobilization in the absence of G-CSF. However, addition of G-CSF further enhanced the mobilizing effects of GPC100 with or without propranolol. Whether propranolol enhances mobilization by the combination treatment of GPC100 and G-CSF remains unclear. In multiple myeloma patients, propranolol was shown to inhibit the molecular risk markers in hematopoietic stem cell transplant, a phenomenon that is currently being investigated in mice by evaluating changes in the inflammatory cytokines following propranolol treatment. The studies disclosed here were performed in naïve or non-tumor bearing mice, which may not have the stress response present in tumor-bearing mice. Future studies will investigate the combination blockade of beta adrenergic and CXCR4 signaling in tumor-bearing C57/BL6 mice to measure stem cell mobilization.

Increased lymphocyte mobilization in propranolol-treated groups was observed in all experiments, which may have the clinical relevance as stated below. A clinical study showed that high T-cell content was associated with rapid hematopoietic reconstitution, decreased relapse, and increased disease-free survival in patients receiving peripheral blood stem cell transplants compared to those receiving the bone marrow transplants (Stem Cell Trialists' Collaborative Group J Clin Onc 2005). Similarly, in both non-human primates and cancer patients, a single injection of the AMD3100 resulted in enhanced lymphocyte count in peripheral blood that included effector T cells and regulatory T cells, which are associated with GVHD-protective properties (Kean et al Blood 2014, Greef et al Blood 2014). A sufficient amount of T lymphocytes is critical in the manufacturing process of CAR-T cells. Some CAR-T products that are being clinically investigated or are commercially available rely on autologous patient-derived T cells. T cells from patients might be insufficient in number or affected by several lines of pretreatment and/or actual disease related treatment (for example, progressive AML) (Fesnak et al Transfus Med Rev 2016). This suggests that lymphocyte mobilization is significant both for allogenic hematopoietic stem cell transplant to reduce GVHD risk, as well as for strategies designed to mobilize both effector and regulatory lymphocyte populations for adoptive cellular therapies.

Previous studies have documented CXCL12/CXCR4-mediated lymphocyte homing in the bone marrow, lymph nodes, high endothelial venules, small blood vessels, thymus, and gastrointestinal tract (Bunting et al Immunol Cell Biol 2011). It has also been reported that beta 2 adrenergic receptors interact with CXCR4 to promote retention of lymphocytes in the lymph nodes (Nakai et al JEM 2014). Hence, increased trafficking of lymphocytes into peripheral blood following blockade of both CXCR4 and beta-adrenergic receptor signaling is expected. The phenotypic profile of immune cells including the lymphocytes that are mobilized by propranolol and GPC100 combination treatments is being further investigated. The results from the study disclosed herein will provide more information on the type of lymphocyte subsets that can be harvested by GPC100 and propranolol combination treatment and their importance in therapeutic development.

### Example 2. Pretreatment with Beta-Adrenergic Receptor Antagonist Propranolol Enhances Stem Cell Mobilization by CXCR4 Antagonist Burixafor (GPC100)

### Materials & Methods and Study Design

The subjects of the study were C57/BL6 Female mice. Peripheral blood was collected 2 hours after vehicle or GPC100, and 1 hour after AMD3100 by terminal cardiac puncture. Complete blood count was determined by hematology analyzer.

TABLE 7

| | Dose | |
|---|---|---|
| Drug | (mg/kg), | |
| combinations | Route | Frequency |
| PBS | 0, SC | QD × 7 days |
| PBS | 0, IV | QD, single dose |
| GPC100 | 30, IV | QD, single dose |
| AMD3100 | 5, SC | QD, single dose |
| Propranolol | 20, IP | QD × 7 days |
| G-CSF | 0.1, SC | BID × 5 days |
| AMD3100 | 5, SC | QD, single dose12 |
| | | h post-G-CSF |

### Results

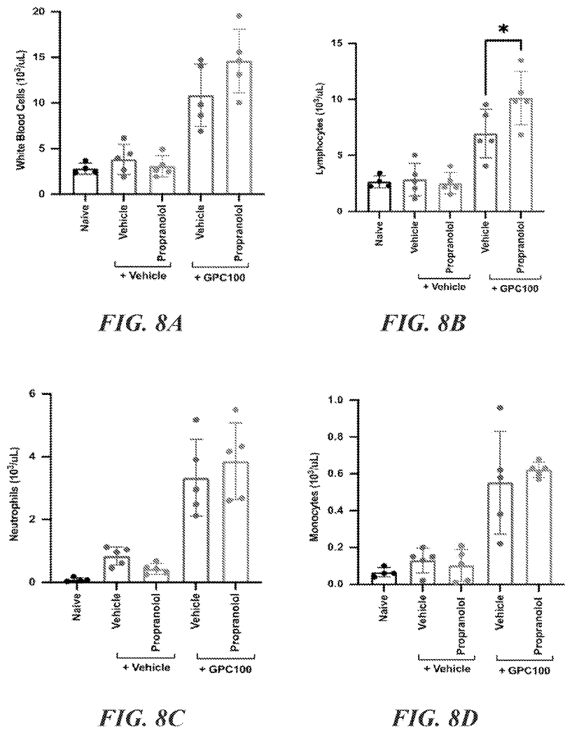

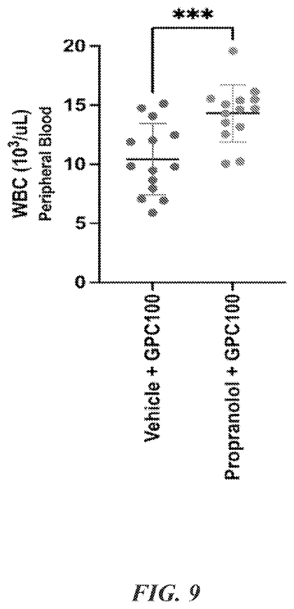

Propranolol was observed to cause and increase in GPC100-induced mobilization. See Table 3. Propranolol alone did not alter blood counts. This is the first study showing enhancement in mobilization by propranolol pretreatment (FIGS. 8A-D). Furthermore, a significant increase in GPC100-induced mobilization after propranolol pretreatment was also observed in a total of 3 studies (FIG. 9).

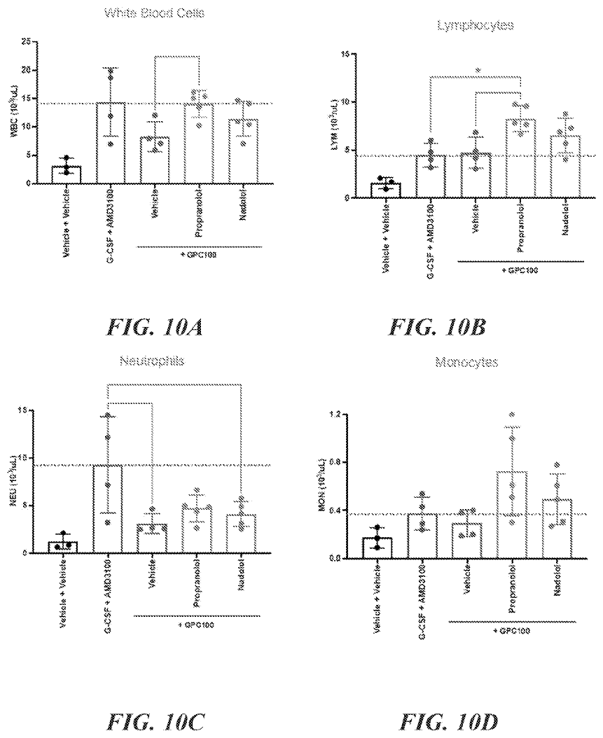

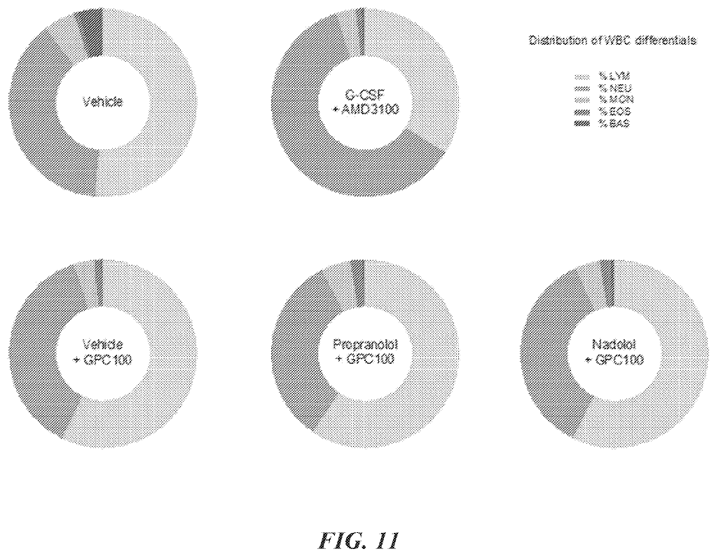

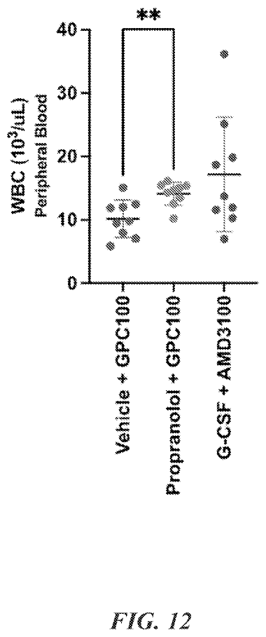

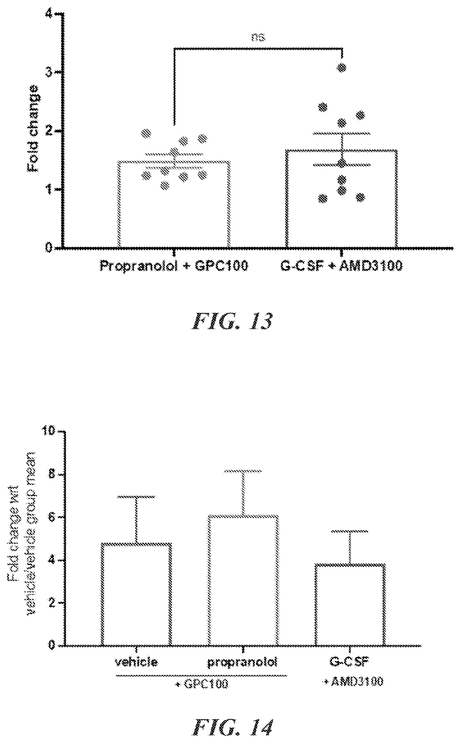

Propranolol-induced increase in mobilization was comparable with the current standard of care in a preclinical model. See Table 3, Table 5, and Table 8. It was observed that an increase in mobilized WBCs from propranolol pretreatment was mainly due to lymphocytes, whereas the SOC regimen mainly mobilized neutrophils (FIGS. 10A-D and FIG. 11). A large variation was observed in the SOC group in both studies, with only SOC also resulting in a reduced number of platelets (FIG. 12 and FIG. 13).

TABLE 8
Complete blood count analysis indicated that in mice treated
with GPC100 and/or propranolol, no change in the number of platelets,
RBCs or hemoglobin levels was observed compared to those treated
with the standard of care treatment or vehicle.

| | Platelets | Hemoglobin | RBC |
|---|---|---|---|
| Treatment | (10³ /uL) | (g/dL) | (10⁶ /uL) |
| Vehicle + Vehicle | 682 ± 494 | 11.8 ± 4.2 | 7.8 ± 2 |
| Vehicle + GPC100 | 777 ± 246 | 11 ± 2.7 | 7.4 ± 1.8 |
| Propranolol + GPC100 | 843 ± 298 | 12.3 ± 1.8 | 8.2 ± 1.3 |
| G-SCF + AMD3100 | 545 ± 213 | 13.9 ± 1.2 | 9.2 ± 0.94 |

* Data from 2 studies

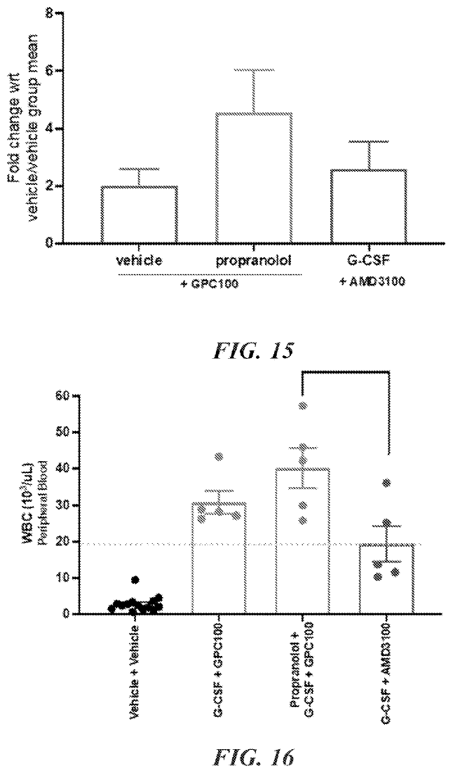

In a determination of hematopoietic stem cell mobilization with the dosing regimen by flow cytometry, no significant difference from standard of care was observed. In mice, hematopoietic stem cells are devoid of lineage markers (Lin-) and express Sca1 and cKit markers (LSK cell profile). CXCR4 is also expressed on hematopoietic stem cells. Data are shown for LSK Cells (FIG. 14) and for Lin−CXCR4+ cells (FIG. 15).

To summarize, single intravenous administration of GPC100/Burixafor was observed to cause rapid increase in circulating WBCs—an indication of stem cell mobilization. Furthermore, CBC analyses from 3 mobilization studies showed that 7-day pretreatment with propranolol enhanced GPC100-induced mobilization. Mobilization from propranolol and GPC100 combination pretreatment was at comparable levels with G-CSF and AMD3100 combination treatment. G-CSF+AMD3100 combination was observed to mobilize more neutrophils, whereas beta blocker+GPC100 combination was observed to mobilize more lymphocytes. Further determination of hematopoietic stem cell mobilization by flow analysis will provide additional insights.

### Example 3. Combination Blockade of CXCR4 and Beta-Adrenergic Receptor Signaling Pathways Induces Stem Cell Mobilization Comparable with the Current Standard of Care

The combined blockade of the two signaling pathways is investigated for its ability to drive CXCR4 and beta-adrenergic receptors. The CXCR4 blockade will be determined by administration of both Burixafor and Plerixafor. The effects of a combination of propranolol+Plerixafor AND propranolol+G-CSF+Plerixafor will also be studied.

### Example 4. Addition of Propranolol/Beta Blockers Improves Stem Cell Mobilization by the Combination Treatment with Burixafor (GPC100) and G-CSF—Triple Combination

### Study Design

New groups were added to this study to determine if propranolol improves response to G-CSF and GPC100 combination. The dosing regimen is given in Table 9 and Table 10. GPC-100 & G-CSF resulted in a higher number of mobilized circulating WBCs and progenitor cells as compared to AMD3100 & G-CSF. The triple combination resulted in highest number of mobilized WBCs and progenitor cells. G-CSF was administered two times daily for five days at 0.1 mg/kg, SC; twelve-hours after the last injection of G-CSF, GPC100 was administered alone at 30 mg/kg, IV. Samples were collected 2 hours after GPC100 administration (Table 9). Propranolol was administered once daily for 7 days; G-CSF was administered twice daily for five days starting on the second day; twelve hours after the last injection of G-CSF, GPC100 was co-administered with propranolol; samples were collected 2 hours after GPC100 administration (Table 10).

TABLE 9
Title: Dosing regimen for G-CSF
and GPC100 combination treatment

| | Day | Day | Day | Day | Day | Day | Day |
|---|---|---|---|---|---|---|---|
| Drug | 1 | 2 | 3 | 4 | 5 | 6 | 7 |

| | | | | | | |
|---|---|---|---|---|---|---|
| G-CSF BID | + | + | + | + | + | |
| 100 ug/kg/ | | | | | | |
| injection SC | | | | | | |
| GPC100 | | | | | | + |
| 30 mg/kg IV | | | | | | |
| (12 h after | | | | | | |
| D6 G-CSF) | | | | | | |

TABLE 10
Title: Dosing regimen for the combination
treatment of propranolol, G-CSF and GPC100

| | Day | Day | Day | Day | Day | Day | Day |
|---|---|---|---|---|---|---|---|
| Drug | 1 | 2 | 3 | 4 | 5 | 6 | 7 |
| Propranolol | + | + | + | + | + | + | + |
| 20 mg/kg IP | | | | | | | |
| G-CSF BID | | + | + | + | + | + | |
| 100 ug/kg/ | | | | | | | |
| injection SC | | | | | | | |
| GPC100 | | | | | | | + |
| 30 mg/kg IV | | | | | | | |
| (12 h after | | | | | | | |
| D6 G-CSF) | | | | | | | |

### WBC Mobilization in G-CSF Combination Studies

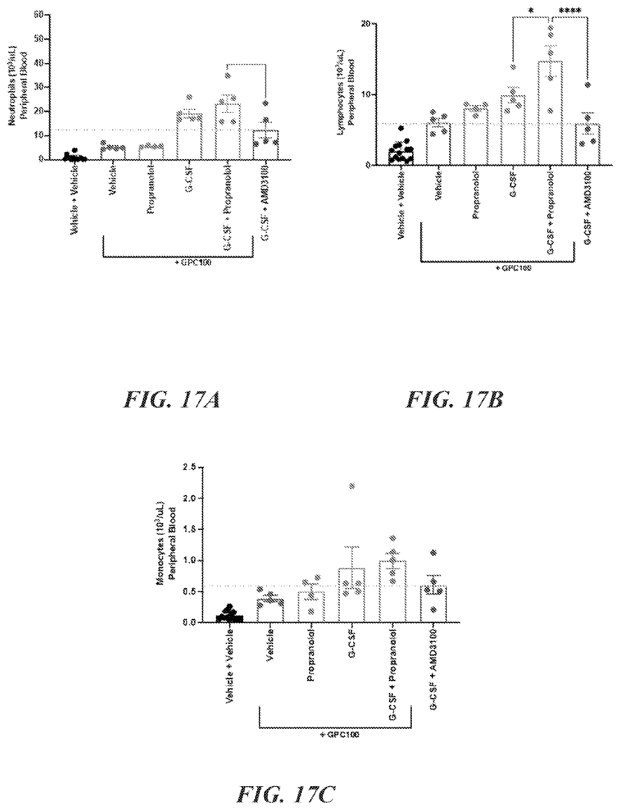

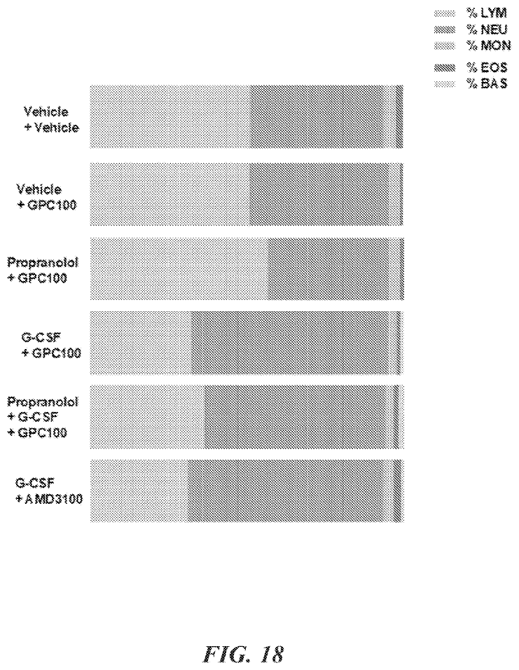

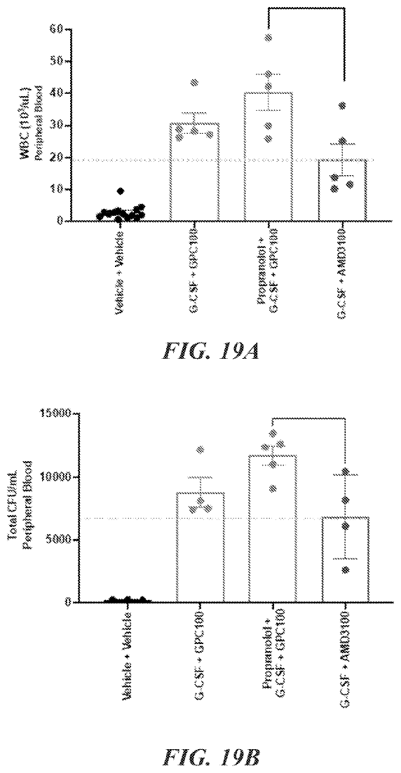

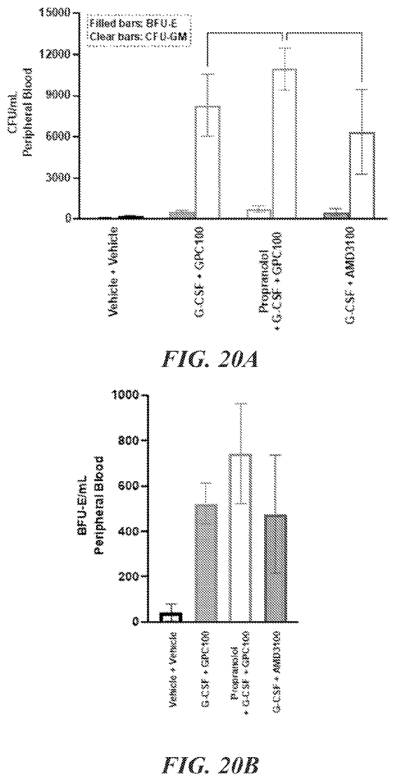

Addition of propranolol to the combination of G-CSF and GPC100 caused maximum mobilization of WBCs, with a significant increase in mobilization compared to SOC or G-CSF and GPC100 combination treatment observed (FIG. 16 and FIGS. 17A-C). Subsequently, changes in leukocyte subsets with treatment were studied. It was observed that GPC100 in combination with propranolol increased lymphocytes, while GPC100 in combination with G-CSF increased Neutrophils (FIG. 18). Increased Colony Formation Unit Assay was only observed in groups with G-CSF (FIG. 19B). GPC-100 & G-CSF resulted in higher number of mobilized circulating WBCs and progenitor cells compared to AMD3100 & G-CSF (FIG. 19A). The triple combination resulted in the highest number of mobilized WBCs and progenitor cells (FIGS. 19A-B and FIGS. 20A-B).

To summarize, G-CSF combination treatment with Burixafor mobilized more WBCs and hematopoietic progenitor cells in the peripheral blood compared to the combination treatment with AMD3100. Addition of 7-day propranolol to the combination treatment of G-CSF and GPC100 resulted in the maximum number of mobilized WBCs, as well as mobilized hematopoietic progenitor cells in the colony formation assay.

### Example 5. Addition of Propranolol/Beta Blockers Improves Stem Cell Mobilization by the Combination Treatment with CXCR4 Antagonists (e.g., Burixafor, Plerixafor) and G-CSF

The combined blockade of the two signaling pathways will be studied for its ability to drive CXCR4 and beta-adrenergic receptors in combination with G-CSF for stem cell mobilization. The CXCR4 blockade will be determined by administration of both Burixafor and Plerixafor. with the combination of propranolol+Plerixafor AND propranolol+G-CSF+Plerixafor will be studied.

### Example 6. In Vivo Pharmacology

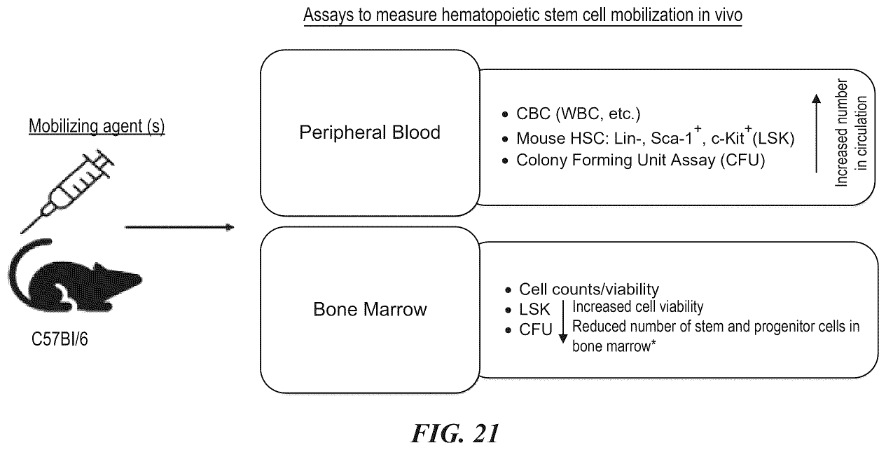

A mouse study design was implemented to investigate the impact of CXCR4 and B2AR blockade on HSC mobilization. Bone marrow replenishes itself in response to cells leaving, hence the number of cells in the bone marrow may not counted as decreased at the time of sample collection. Studies focused on mobilization into the peripheral blood. (FIG. 21 and Table 11).

TABLE 11
Combination studies with beta blockers and GPC100 for
stem cell mobilization in mice at GPCR Therapeutics

| | Beta. | | | | | |
|---|---|---|---|---|---|---|
| | Blockers | SOC | | | Flow | |
| | (7 d) + | (G-CSF + | G-CSF | CBC | Cytometry | CFU |
| Study ID | GPC100 | AMD3100) | Combination | analysis | LSK cells | Assay |
| GPCR003 | Propranolol | | | ✓ | | |
| U2109 | Nadolol | | | | | |
| “Study 1” | ICI-118,551 | | | | | |
| GPCR005 | Propranolol | | | ✓ | ✓ | |
| U2210 | Nadolol | | | | | |
| “Study 2” | | | | | | |
| GPCR006 | Propranolol | ✓ | | ✓ | ✓ | |
| U2111 | Nadolol | | | | | |
| “Study 3” | | | | | | |
| GPCR007 | Propranolol | ✓ | ✓ | ✓ | | ✓ |
| U2201 | | | | | | |
| “Study 4” | | | | | | |
| GPCR008 | Propranolol | ✓ | ✓ | ✓ | ✓ | |
| U2202 | | | | | | |
| “Study 5” | | | | | | |
| GPCR010 | Propranolol | ✓ | ✓ | ✓ | ✓ | |
| “Study 6” | | | | | | |

Role of CXCR4 antagonists in stem cell mobilization. Binding of the chemokine CXCL12 to its receptor CXCR4 plays an essential role in homing and retention of HSC in the bone marrow. Preclinical studies showed that a single intravenous administration of the CXCR4 antagonist GPC100 resulted in the rapid increase of circulating WBCs in C57/B16 and Balb/c mice, which is an indication of stem cell mobilization. CXCR4 antagonists like Plerixafor (AMD3100) and Burixafor (GPC100) are clinically approved in the U.S and Europe for use in combination with G-CSF for hematopoietic stem cell mobilization and subsequent autologous stem cell transplant in Non-Hodgkin's Lymphoma and multiple myeloma patients. G-CSF regimen involves repeated multi-day injections and is associated with adverse side effects like severe bone pain. Poor mobilization has also been reported in up to 40% patients. Therefore, an alternate approach to improve hematopoietic stem cell mobilization by CXCR4 antagonists is needed.

There is a need to improve stem cell mobilization for several reasons, including the following. ASCT is being increasingly used to treat hematological malignancies. However, successful ASCT in lymphoma and MM patients is often hindered by poor mobilization with at least 15% of patients failing to produce the target cell dose of >2×10⁶ CD34⁺ cells/kg required to proceed with ASCT (Olivieri et al. 2012). Newer therapies for MM patients approved in recent years may also have a negative impact on mobilization. For example, recent studies have shown that MM patients receiving daratumumab induction before ASCT have poorer mobilization (Hulin et al. 2021). Daratumumab use is also associated with an increased rate of neutropenic fever, leading to increased antibiotic usage and prolonged hospitalization (Papaiakovou et al. 2021). This further increases the patient burden, which is another factor to consider in the treatment of MM patients. Compared to patients with other hematological malignancies, MM patients have been found to have a higher symptom burden and worse Health-Related Quality of Life (HRQoL) (Johnsen et al. 2009).

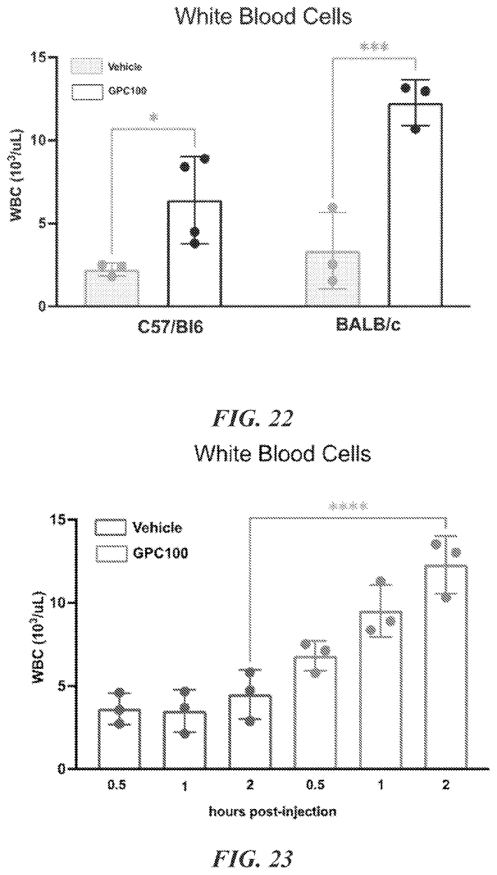

GPC100-induced mobilization in mice. (FIG. 22). A single intravenous (IV) administration of GPC-100 (30 mg/kg), a potent and selective antagonist of CXCR4, resulted in the rapid increase of circulating white blood cell counts (WBCs) in C57/B16 and Balb/c mice, which is reflective of stem cell mobilization. In order to determine the time course of GPC-100-induced mobilization, GPC-100 (30 mg/kg) was administered intravenously in naïve C57/B16 mice, and peripheral blood was collected at time points 0.5-, 1- and 2-hours post-injection in different groups. Time dependent increase in WBC count was observed and a 2-hour post-injection sample collection was selected for subsequent studies. C57/B16 mice were selected over Balb/c for future studies as HSC mobilization has been critically evaluated in this mouse strain (Broxmeyer et al 2005).

It was observed that GPC-100 (30 mg/kg, IV) alone induces time-dependent WBC Mobilization (FIG. 23). A future study will employ sample collection at 2 hours post-GPC administration. The planned time-course study will be as follows: 0.5, 1, 2, 3, 4 hours post-GPC100 administration. Dose response with IV GPC100 administration will be performed (doses TBD).

Rationale for using beta blockers to enhance CXCR4-induced mobilization. Bone marrow is highly innervated by the sympathetic nervous system. Traumatic stress in humans and rodent models have shown persistently elevated levels of norepinephrine, a ligand of beta-adrenergic receptors, which is associated with bone marrow dysfunction (Bible et al 2014, Bible et al 2015a, Bible et al 2015b). Thus, future studies will evaluate the potential of beta blockers to improve GPC100-induced mobilization by restoring the bone marrow function. In the studies presented elsewhere, seven-day intraperitoneal administration of non-selective beta blockers propranolol (20 mg/kg) and nadolol (5 mg/kg) or selective beta-2 receptor antagonist ICI-118,551 (5 mg/kg) alone did not affect the total blood cell counts. This dose of propranolol will be chosen for future studies involving beta-adrenergic blockade.

### Example 7. White Blood Cell Mobilization Induced by GPC-100+/−Beta Blockers

A study was conducted with the dosing schedule as shown in Table 12 and Table 13. Propranolol, nadolol or ICI-118,551 was administered once daily for 7 days. GPC100 was co-administered with propranolol on day 7 (Table 12). Propranolol, nadolol or ICI-118,551 was administered once daily for 7 days; vehicle was co-administered intravenously with propranolol on day 7 (Table 13).

TABLE 12
Dosing Schedule for beta blocker
and GPC100 combination treatment

| | Day | Day | Day | Day | Day | Day | Day |
|---|---|---|---|---|---|---|---|
| Drug | 1 | 2 | 3 | 4 | 5 | 6 | 7 |
| Propranolol | + | + | + | + | + | + | + |
| 20 mg/kg IP | | | | | | | |
| OR Nadolol | | | | | | | |
| 5 mg/kg IP OR | | | | | | | |
| ICI-118,551 | | | | | | | |
| 5 mg/kg IP | | | | | | | |
| GPC100 | | | | | | | + |
| 30 mg/kg IV | | | | | | | |

TABLE 13
Dosing Schedule for beta blocker treatment

| | Day | Day | Day | Day | Day | Day | Day |
|---|---|---|---|---|---|---|---|
| Drug | 1 | 2 | 3 | 4 | 5 | 6 | 7 |
| Propranolol | + | + | + | + | + | + | + |
| 20 mg/kg IP | | | | | | | |
| OR Nadolol | | | | | | | |
| 5 mg/kg IP OR | | | | | | | |
| ICI-118,551 | | | | | | | |
| 5 mg/kg IP | | | | | | | |
| Vehicle IV | | | | | | | + |

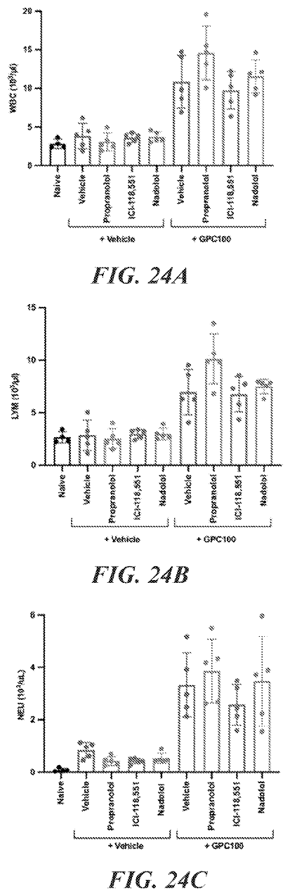

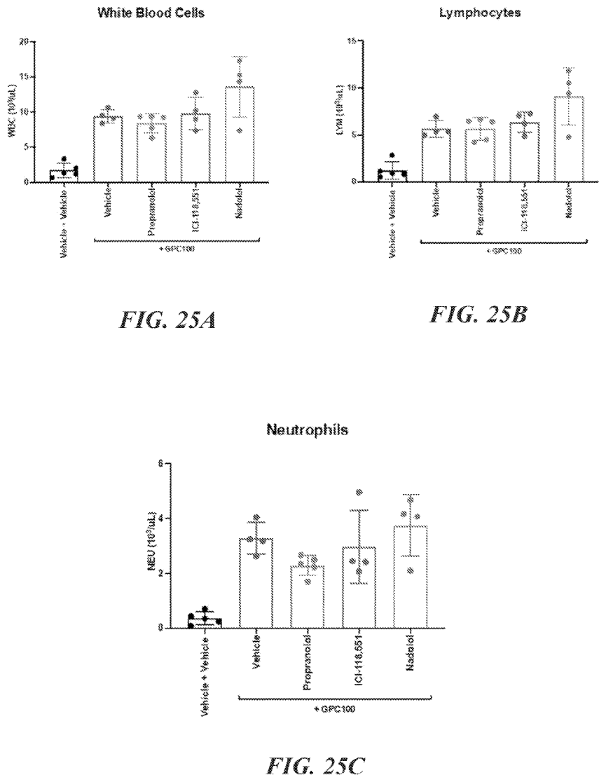

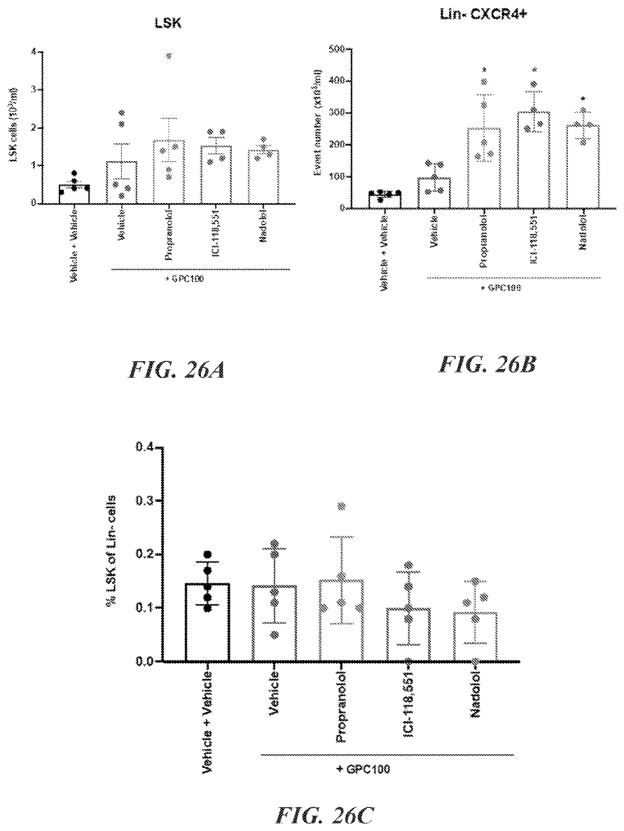

White Blood Cell Mobilization Induced by GPC-100+/−Beta Blockers. 7-day administration of propranolol enhanced GPC100 induced mobilization but had no effect on blood counts when administered alone (FIGS. 24A-C). It was observed that Beta blocker administered for 3-days or concurrently with GPC100 did not alter blood counts (data not shown). Future studies will be conducted to: (1) Confirm that hematopoietic stem cells are being mobilized; (2) Mouse hematopoietic stem cells are devoid of lineage markers (lin−) and express SCA1 and cKit. Hence, LSK cell profile determined by Flow cytometry (Reach Bio); (3) Repeat CBC. Studies were conducted to determine stem cell mobilization. It was observed that Nadolol enhanced GPC100-induced mobilization (FIGS. 25A-C). Additionally, 7-day beta-blocker administration with a single GPC100 did not seem to increase LSK and Lin−CXCR4+ cells (FIGS. 26A-26C). Future studies will repeat the experiment and add a standard of care group.

A study was performed to study compare with G-CSF+AMD3100 (Table 14). Propranolol (20 mg/kg IP) was administered once daily for 7 days. On day 7, GPC100 (30 mg/kg IV) was co-administered with propranolol. Peripheral blood was collected 2 hours post-injection by cardiac puncture. This outcome was compared with the current standard of care for mobilization i.e., the combination treatment with G-CSF and AMD3100 (Plerixafor). G-CSF (0.1 mg/kg SC) was administered for 5 days two times a day, followed by a single injection of AMD3100 (5 mg/kg SC) on day 6 after 12 hours. Peripheral blood was collected 1-hour post-AMD3100 based on the literature reports (Hoggatt et al 2018).

TABLE 14
Dosing regimen based on literature

| | Day | Day | Day | Day | Day | Day | Day |
|---|---|---|---|---|---|---|---|
| Drug | 1 | 2 | 3 | 4 | 5 | 6 | 7 |

| | | | | | | |
|---|---|---|---|---|---|---|
| G-CSF BID | + | + | + | + | + | |
| 100 ug/kg/ | | | | | | |
| injection SC | | | | | | |
| AMD3100 | | | | | | + |
| 5 mg/kg SC | | | | | | |

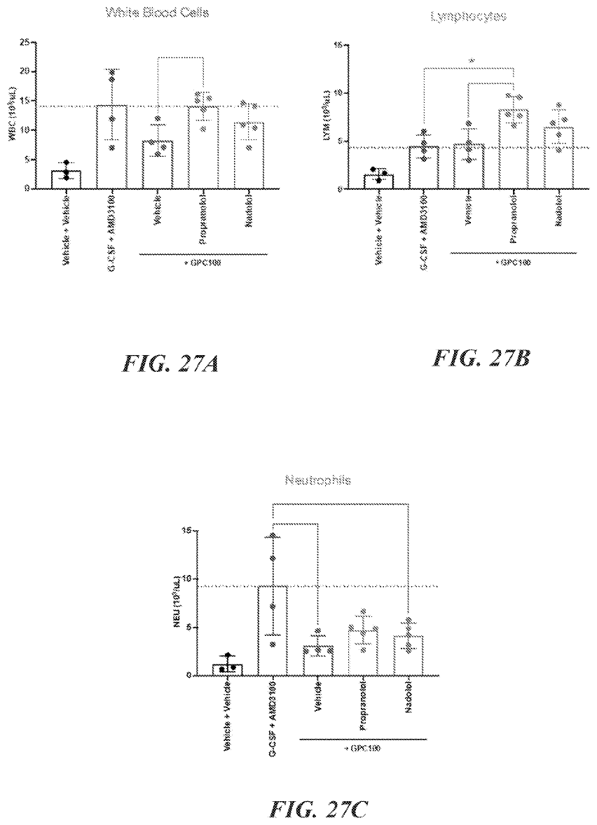

Propranolol was observed to enhance GPC100-induced mobilization (FIGS. 27A-C). This effect was comparable with the standard of care (G-CSF+AMD3100/Plerixafor). The Propranolol+GPC100 combination was observed to mobilize more lymphocytes. It was also observed that SOC mobilized more neutrophils (G-CSF driven).

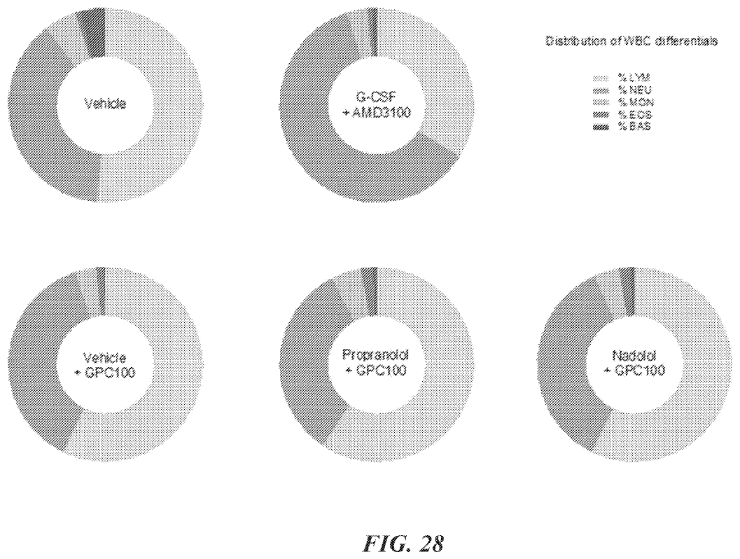

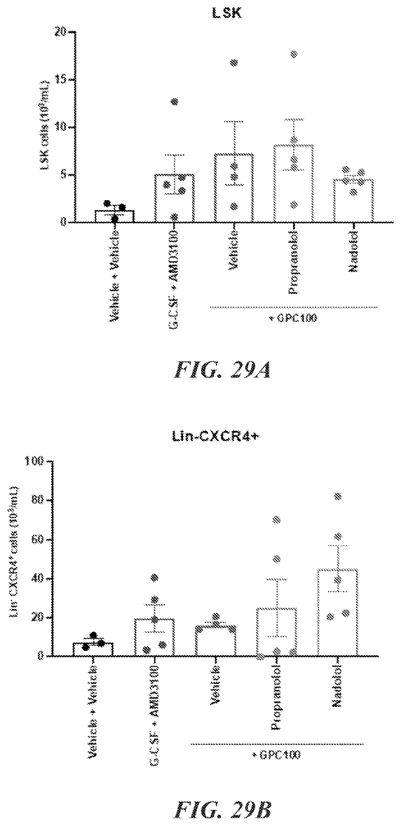

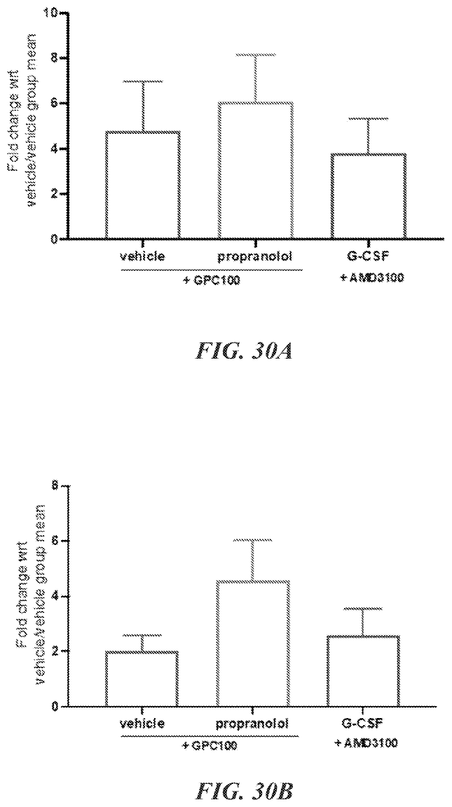

A study was performed with the observation that lymphocytes increased with GPC100 and beta blockers, while neutrophils increased with G-CSF+AMD3100 (FIG. 28). The fold change of combined studies 2 and 3 is shown in FIGS. 29A-B, for LSK (FIG. 30A) and Lin−CXCR4+ (FIG. 30B). For LSK flow, propranolol+GPC100 combination was comparable with SOC. Future studies will repeat this experiment with more blood volume and added G-CSF.

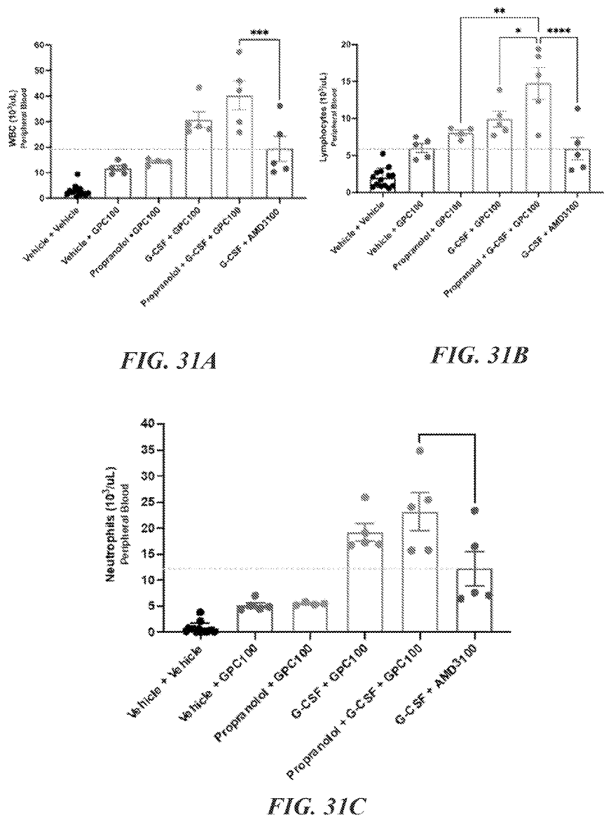

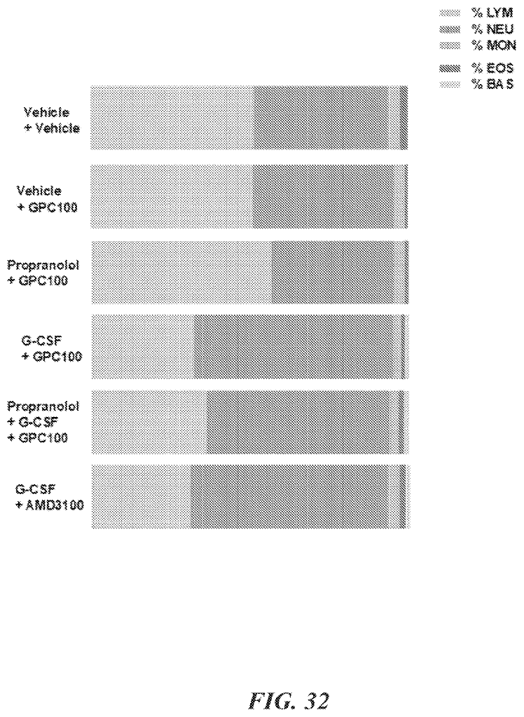

An experiment was performed to study the addition of triple combination with G-CSF (Table 15). The triple combination produced the best results (FIGS. 31A-C and FIG. 32) and Propranolol+GPC100 was comparable with SOC.

TABLE 15
Triple combination dosing schedule.

| | Day | Day | Day | Day | Day | Day | Day |
|---|---|---|---|---|---|---|---|
| Drug | 1 | 2 | 3 | 4 | 5 | 6 | 7 |
| Propranolol | + | + | + | + | + | + | + |
| 20 mg/kg IP | | | | | | | |
| G-CSF BID | | + | + | + | + | + | |
| 100 ug/kg/ | | | | | | | |
| injection SC | | | | | | | |
| GPC100 | | | | | | | + |
| 30 mg/kg IV | | | | | | | |

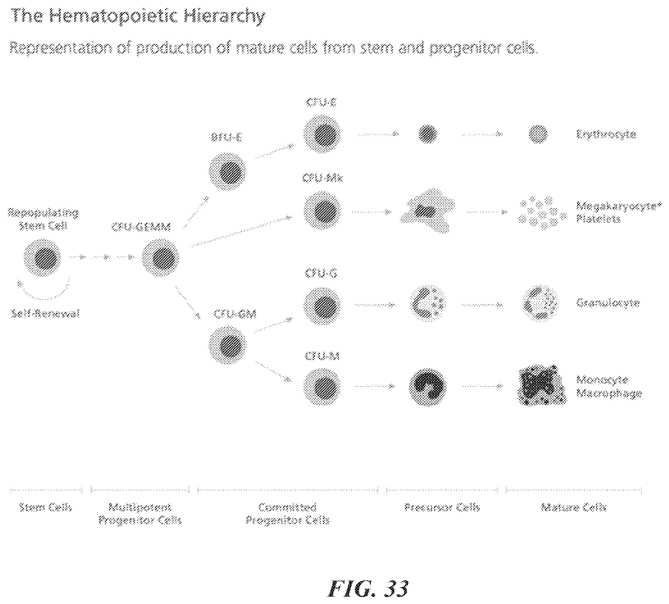

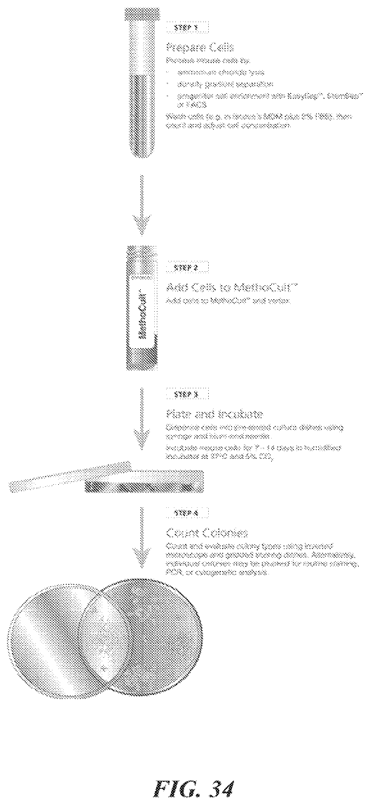

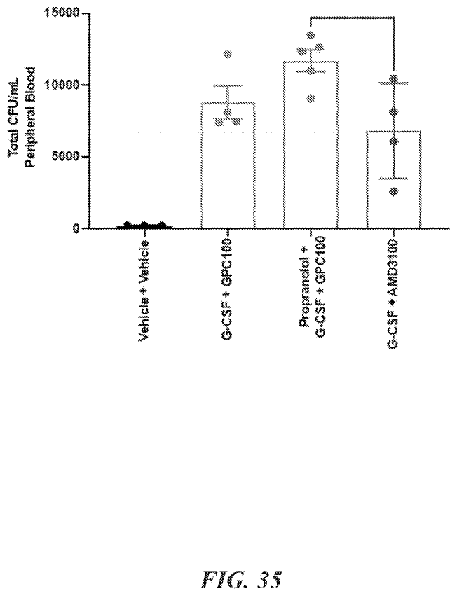

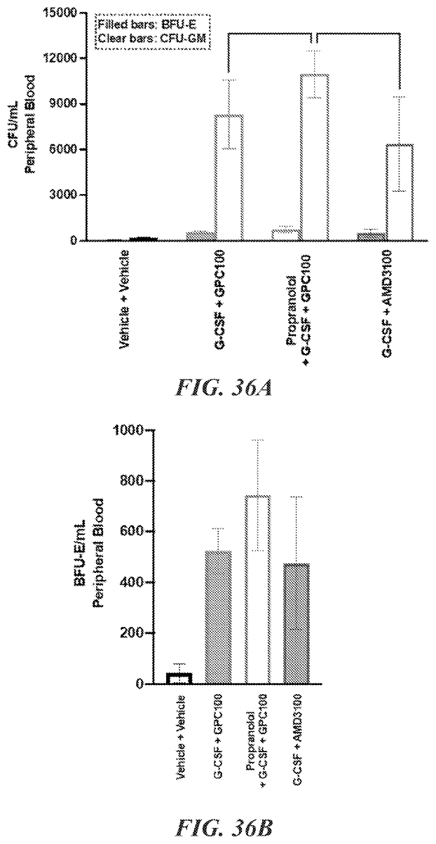

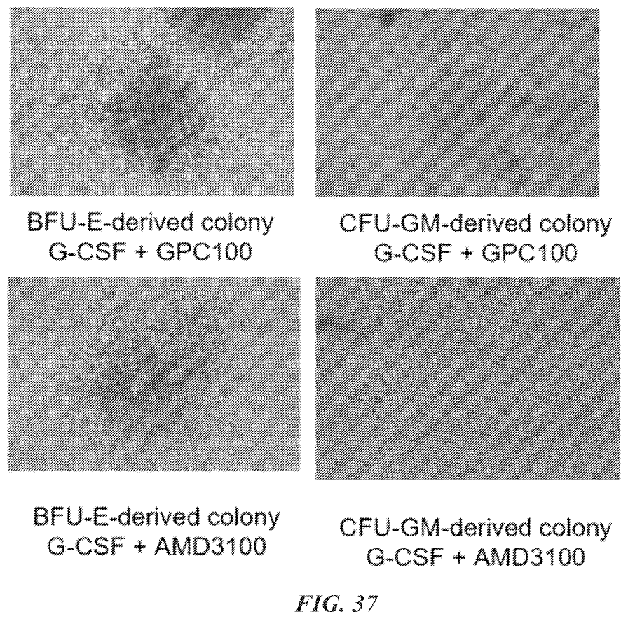

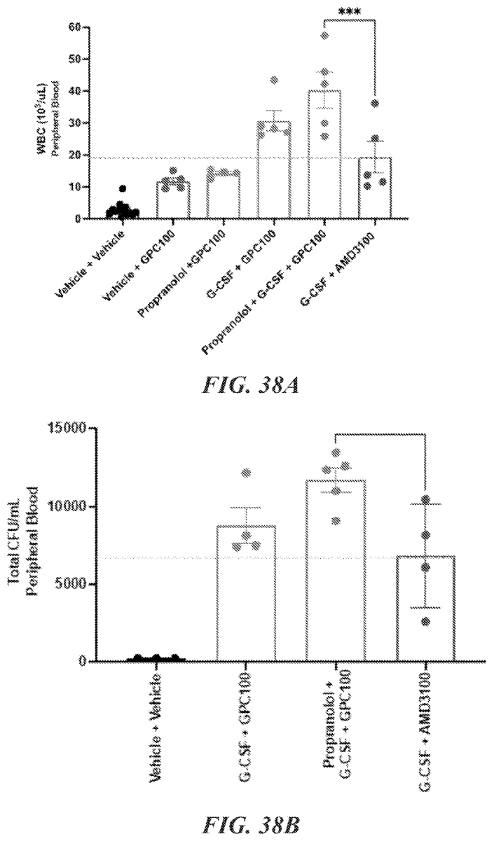

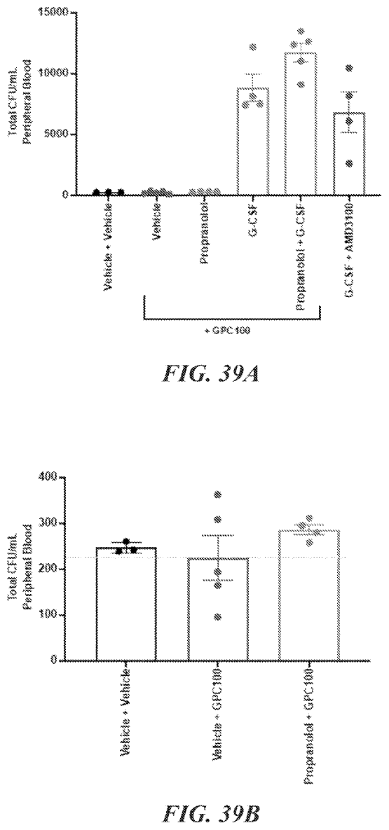

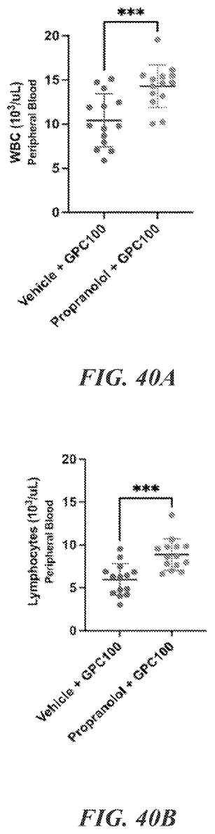

A Colony Forming Unit Assay was performed (FIG. 33 and FIG. 34). The CFU assay is based on the ability of hematopoietic progenitors to proliferate and differentiate into colonies in a semi-solid media in response to cytokine stimulation. Number and types of colonies counted in a CFU assay provide information about the frequency and types of progenitor cells present in the original cell population and their ability to proliferate and differentiate. The triple combination mobilized the highest number of progenitor cells (FIG. 35, FIGS. 36A-36B and FIG. 37). Furthermore, the triple combination was associated with the maximum increase in circulating WBCs as well as progenitor cells compared to other drug groups (FIGS. 38A-38B). Additionally, GPC100+G-CSF mobilized more WBCs and progenitors compared to AMD3100+G-CSF. No difference was observed between vehicle group vs. GPC100+/−propranolol. The CFU assay was only designed for myeloid progenitors, and not lymphoid (FIGS. 39A-39B). G-CSF was observed to mobilize myeloid progenitors and the assay was observed to be dependent on G-CSF. Data regarding the effect of propranolol on GPC100-induced mobilization from 3 studies (studies 1, 3, 4) are shown in FIGS. 40A-40B. It was observed that Propranolol enhanced GPC100 induced mobilization in 3 studies.

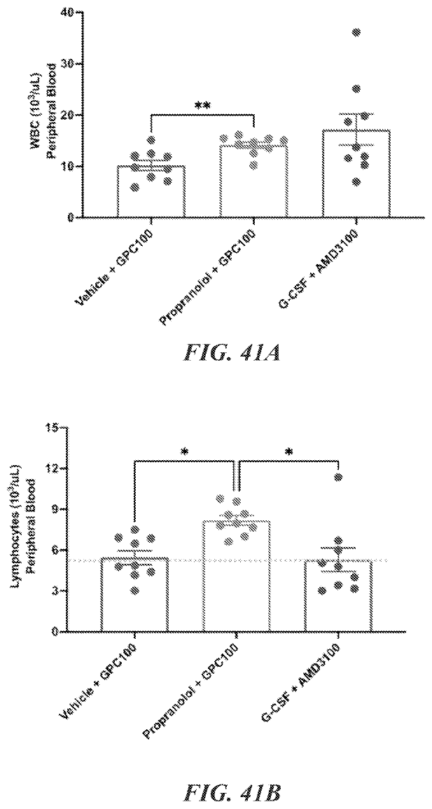

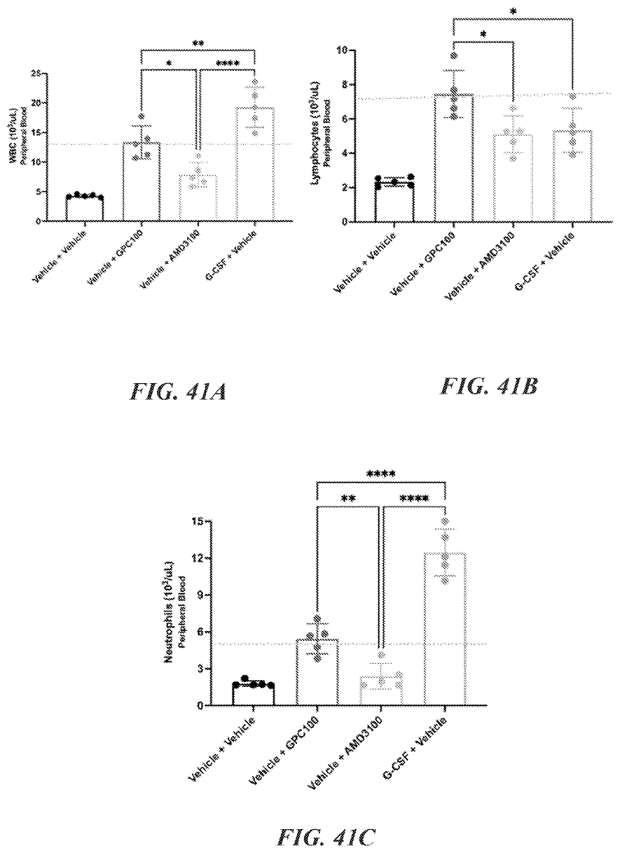

The effect of propranolol on GPC100-induced mobilization compared to the standard of care was studied. Propranolol enhanced GPC100 induced mobilization comparable with SOC (FIGS. 41A-B). Propranolol+GPC100 was observed to mobilize more lymphocytes compared to SOC. The effect of propranolol on GPC100 and AMD3100 induced mobilization was studied with and without the combination with G-CSF (Table 16, Table 17).

TABLE 16

| | | Treatment 1 | | | |
|---|---|---|---|---|---|
| | | Groups 1, 2, 3, | | | |
| | | 5, 6: D 1-D 7 | | Treatment 2 | Dose |
| | | Group 4, 7, 8: | Dose | D 7 for all | (mg/kg) |
| Group | N | D 2-D 6 | (mg/kg) | groups | IV |
| 1 | 5 | Vehicle, IP | 0 | Vehicle, IV | 0 |
| 2 | 5 | Vehicle, IP | 0 | GPC100, IV | 30 |
| 3 | 5 | Vehicle, SC | 0 | AMD3100, SC | 5 |
| 4 | 5 | G-CSF, BID, SC | 0.1 | Vehicle, IV | 0 |
| 5 | 6 | Propranolol, IP | 20 | GPC100, IV | 30 |
| 6 | 5 | Propranolol, IP | 20 | AMD3100, SC | 5 |
| 7 | 6 | G-CSF, SC | 0.1 | GPC100, IV | 30 |
| 8 | 5 | G-CSF, SC | 0.1 | AMD3100, SC | 5 |

TABLE 17

| | | Treatment | Treatment | Treatment |
|---|---|---|---|---|
| Group | N | 1 D 1-D 7 | 2 D 2-D 6 | 3 D 7 |
| 9 | 5 | Propranolol | G-CSF BID, | GPC100 |
| | | 20 mg/kg, IP | 12 h apart, SC | 30 mg/kg, IV |
| 10 | 5 | Propranolol | G-CSF BID, | AMD3100 |
| | | 20 mg/kg, IP | 12 h apart, SC | 5 mg/kg, SC |

GPC100, AMD3100 or G-CSF induced WBC mobilization (single agent) was studied (FIGS. 42A-42C). Maximum mobilization was observed with G-CSF, while GPC100 mobilized more lymphocytes than AMD3100 or G-CSF. Furthermore, GPC100 mobilized more WBCs than AMD3100 and G-CSF mobilized more neutrophils than GPC100 or AMD3100.

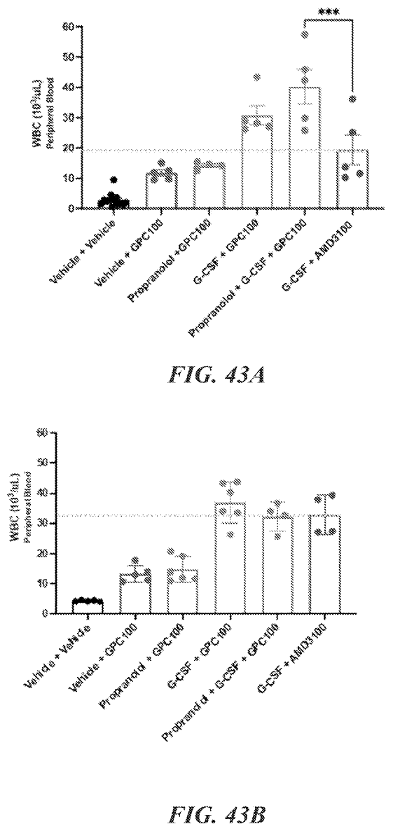

The effect of propranolol on GPC100-induced mobilization was studied in the absence or presence of G-CSF in comparison with standard of care in WBC. The data from Study 4 are shown in FIG. 43A, while the data from Study 5 are shown in FIG. 43B. The effect of the triple combination was not observed in study 5 and the SOC effect was much higher than previously observed.

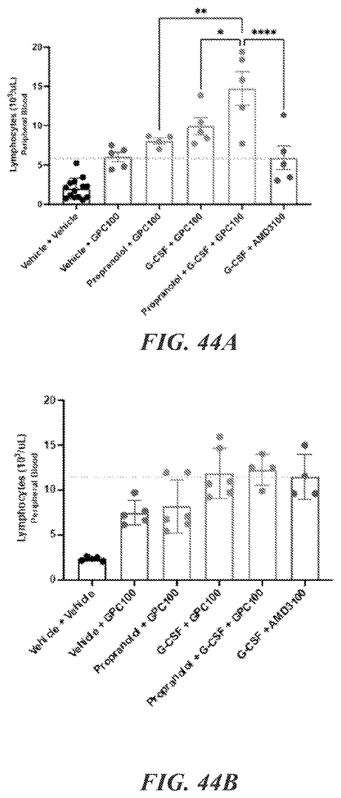

The effect of propranolol on GPC100-induced mobilization was studied in the absence or presence of G-CSF in comparison with standard of care in lymphocytes. The data from Study 4 are shown in FIG. 44A, while the data from Study 5 are shown in FIG. 44B. The effect of the triple combination effect was not observed in study 5 and the SOC effect was much higher than previously observed.

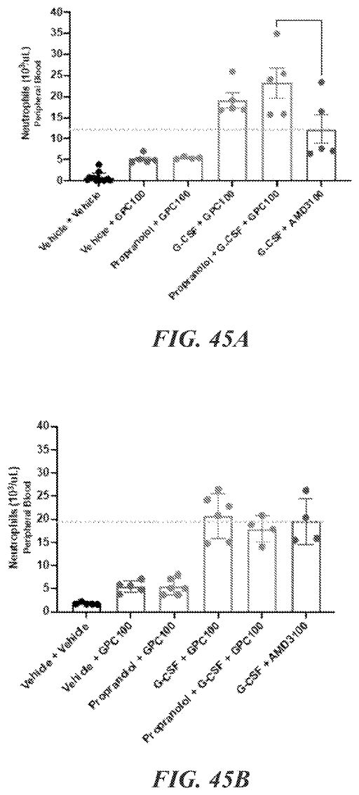

The effect of propranolol on GPC100-induced mobilization was studied in the absence or presence of G-CSF in comparison with standard of care in neutrophils. The data from Study 4 are shown in FIG. 45A, while the data from Study 5 are shown in FIG. 45B. The effect of the triple combination effect was not observed in study 5 and the SOC effect was much higher than previously observed.

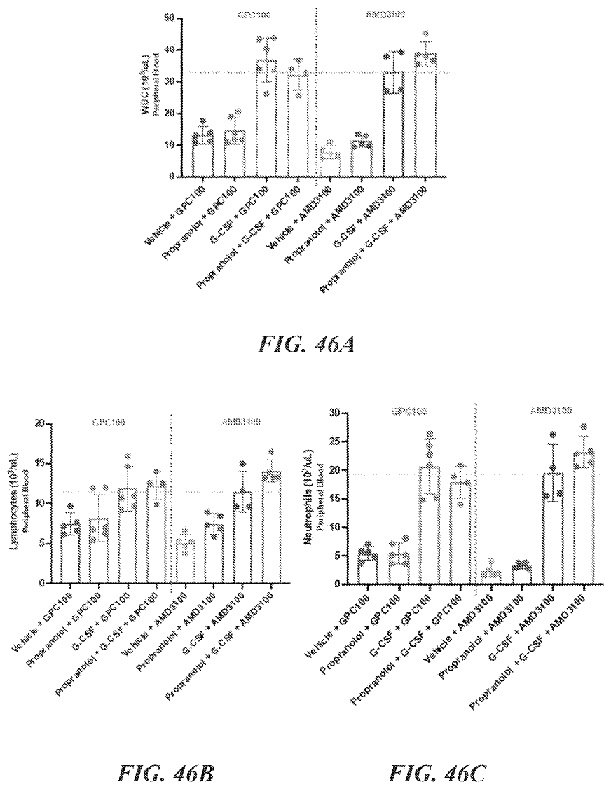

A comparison study between GPC100 and AMD3100 was performed (FIGS. 46A-C). This was the first study to show the effect of propranolol and triple combination with AMD3100. Propranolol was observed to slightly increase AMD3100-induced mobilization of lymphocytes.

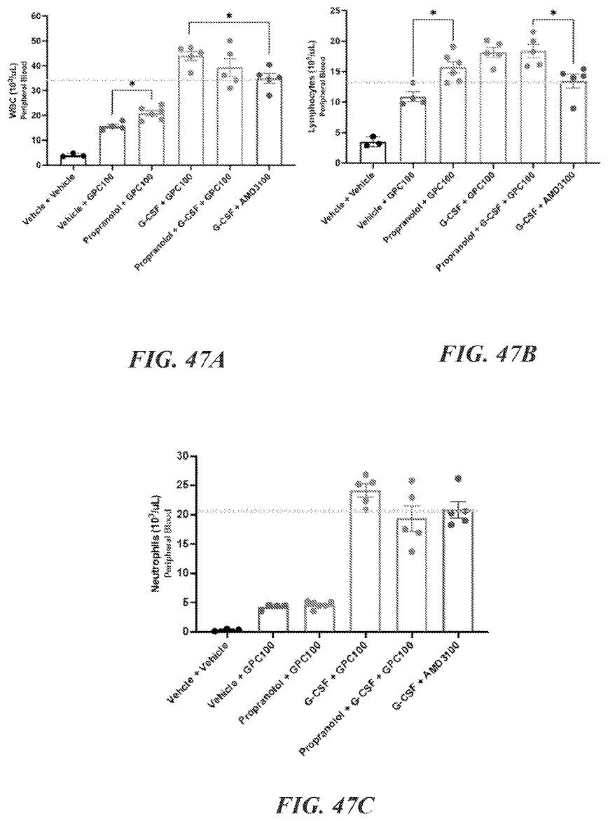

The effect of propranolol on GPC100-induced mobilization with or without G-CSF was studied and a comparison with standard of care performed (FIGS. 47A-C). Propranolol significantly enhanced GPC100-induced mobilization. When combined with G-CSF, GPC100-mobilized more WBCs compared to AMD3100. Propranolol and GPC100 combination resulted in increased circulating lymphocytes at levels similar to the standard of care (G-CSF+AMD3100).

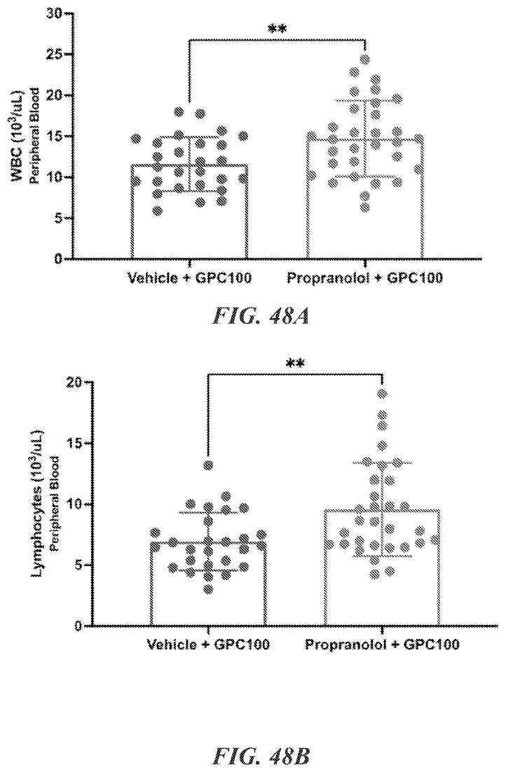

Combined data from all 6 studies are shown in FIGS. 48A-B. 7-day propranolol treatment prior to GPC100 results in significantly enhanced WBC and lymphocyte cell counts in peripheral blood compared to GPC100 alone.

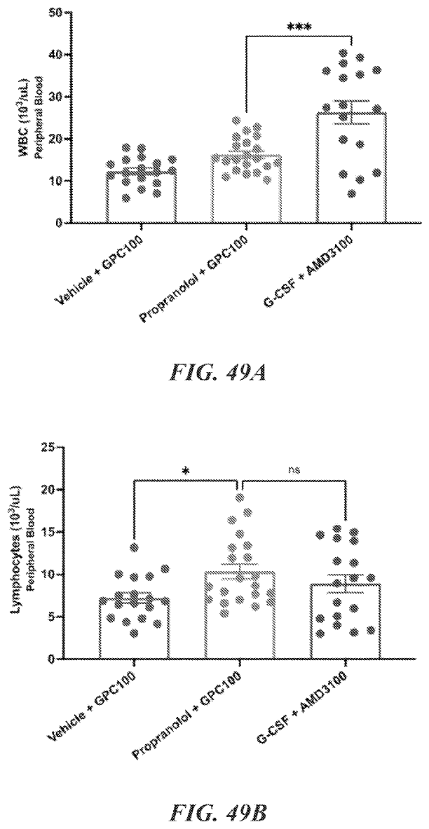

Data from 4 studies in which standard of care group was added are shown in FIGS. 49A-B. It was observed that the standard of care regimen mobilized more WBCs compared to the propranolol and GPC100 combination. However, there was no difference in lymphocyte mobilization. The standard of care group also showed high variability, which reflects the mobilization patient response in the clinic.

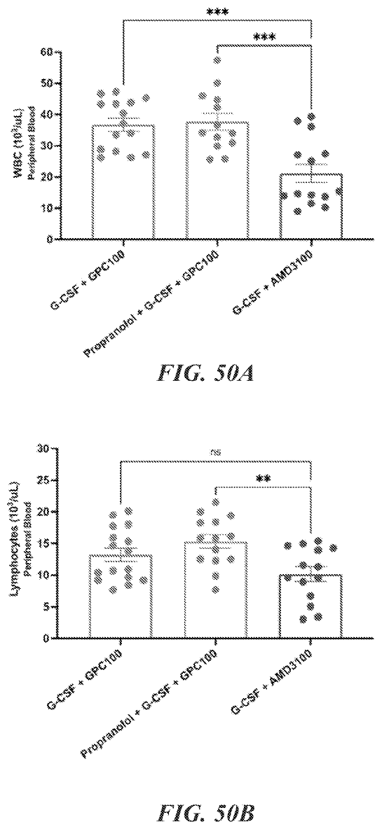

Data from 3 studies in which G-CSF combination group was added are shown in FIGS. 50A-B. It was observed that addition of propranolol to G-CSF and GPC100 combination mobilized significantly more WBC and lymphocytes compared to the standard of care. When combined with G-CSF, GPC100 was observed to mobilize significantly more WBCs, compared to AMD3100. However, with addition of propranolol there was significantly more mobilization of lymphocytes.

### Example 8. Effect of Propranolol on GPC100-Induced Mobilization

### Role of CXCR4 Antagonists in Stem Cell Mobilization

Binding of the chemokine CXCL12 to its receptor CXCR4 plays an essential role in homing and retention of HSC in the bone marrow. Preclinical studies showed that a single intravenous administration of the CXCR4 antagonist GPC100 resulted in the rapid increase of circulating WBCs in C57/B16 and Balb/c mice, which is an indication of stem cell mobilization. CXCR4 antagonists like Plerixafor (AMD3100) and Burixafor (GPC100) are clinically approved in the U.S and Europe for use in combination with G-CSF for hematopoietic stem cell mobilization and subsequent autologous stem cell transplant in Non-Hodgkin's Lymphoma and multiple myeloma patients. G-CSF regimen involves repeated multi-day injections and is associated with adverse side effects like severe bone pain. Poor mobilization has also been reported in up to 40% patients.

### Lymphocyte Mobilization

High T-cell content was associated with rapid hematopoietic reconstitution, decreased relapse, increased disease-free survival in patients receiving peripheral blood stem cell transplants compared to those receiving bone marrow transplants highlighting the importance of lymphocyte mobilization (Stem Cell Trialists' Collaborative Group J Clin Onc 2005). A study in non-human primates showed that single injection of the CXCR4 antagonist AMD3100 resulted in enhanced lymphocyte count in peripheral blood that included effector T cells, as well as Treg and Tem, which are associated with GVHD-protective properties (Kean et al Blood 2014). Similarly, allogeneic stem cell grafts harvested in healthy donors following a single dose of AMD3100 contained higher numbers of both effector and regulatory T-cells as compared to grafts harvested following G-CSF. (Greef et al Blood 2014). This is significant both for allo-HSCT as well as for strategies designed to mobilize both effector and regulatory lymphocyte populations for adoptive cellular therapies. Previous studies have documented CXCL12/CXCR4-mediated lymphocyte homing in the bone marrow, lymph nodes, high endothelial venules, small blood vessels, thymus, and gastrointestinal tract (Bunting et al Immunol Cell Biol 2011).

### Lymphocyte Mobilization for CAT-T Therapies

Efficient leukapheresis providing a sufficient amount of T lymphocytes is a critical step in the manufacturing process of CAR-T cells. Some CAR-T cell products under current investigation are based on allogeneic T cells from healthy donors, while some CAR-T products that are clinically investigated or are commercially available rely on autologous patient derived T cells. T cells from patients might be decreased in number or hampered by several lines of pretreatment and actual disease related treatment (for example, progressive AML) (Fesnak et al Transfus Med Rev 2016).

### Lymphocyte Mobilization & Beta Blockade

Stem cells in the leukapheresis product pose the risk of malignant transformation during the process of genetic modification by viral transduction, indicating the risk that might be posed by stem cell mobilization by G-CSF. It has been reported that beta 2 adrenergic receptors interact with CXCR4 to promote retention of lymphocytes in the lymph nodes (Nakai et al JEM 2014). Hence, this study determined the effects of the combined blockade of beta adrenergic receptor and CXCR4 signaling to increase trafficking of lymphocytes into the peripheral blood.

Study Design. Effect of propranolol on GPC100-induced mobilization (Table 18). C57/BL6 mice received the non-selective beta blocker propranolol (20 mg/kg, IP) once in a day for 7 days. On day 7, GPC100 (30 mg/kg, IV) was co-administered. Blood was collected 2 hours after the drug administration based on the preliminary data showing that maximum mobilization occurred at 2 hours after single intravenous administration of GPC100.

TABLE 18
Dosing Schedule

| | Day | Day | Day | Day | Day | Day | Day |
|---|---|---|---|---|---|---|---|
| Drug | 1 | 2 | 3 | 4 | 5 | 6 | 7 |
| Propranolol | + | + | + | + | + | + | + |
| 20 mg/kg IP | | | | | | | |
| GPC100 | | | | | | | + |
| 30 mg/kg IV | | | | | | | |

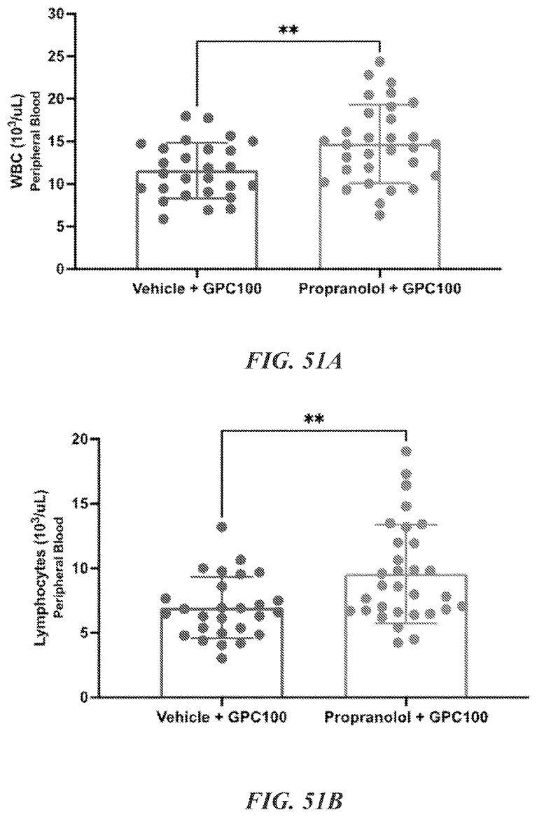

Combined data from all 6 studies are shown in FIGS. 51A-B. 7-day propranolol treatment prior to GPC100 was observed to result in significantly enhanced WBC and lymphocyte cell counts in peripheral blood compared to GPC100 alone.

Study Design. The effect of propranolol on GPC100-induced mobilization in comparison with the standard of care for stem cell mobilization (G-CSF+AMD3100) was studied (Table 19). Propranolol (20 mg/kg IP) was administered once daily for 7 days. On day 7, GPC100 (30 mg/kg IV) was co-administered with propranolol. Peripheral blood was collected 2 hours post-injection by cardiac puncture. This outcome was compared with the current standard of care for mobilization, i.e., the combination treatment with G-CSF and AMD3100 (Plerixafor). G-CSF (0.1 mg/kg SC) was administered for 5 days two times a day, followed by a single injection of AMD3100 (5 mg/kg SC) on day 6 after 12 hours. Peripheral blood was collected 1-hour post-AMD3100 based on the literature reports (Hoggatt et al 2018).

TABLE 19
Dosing regimen based on literature

| | Day | Day | Day | Day | Day | Day | Day |
|---|---|---|---|---|---|---|---|
| Drug | 1 | 2 | 3 | 4 | 5 | 6 | 7 |

| | | | | | | |
|---|---|---|---|---|---|---|
| G-CSF BID | + | + | + | + | + | |
| 100 ug/kg/ | | | | | | |
| injection SC | | | | | | |
| AMD3100 | | | | | | + |
| 5 mg/kg SC | | | | | | |

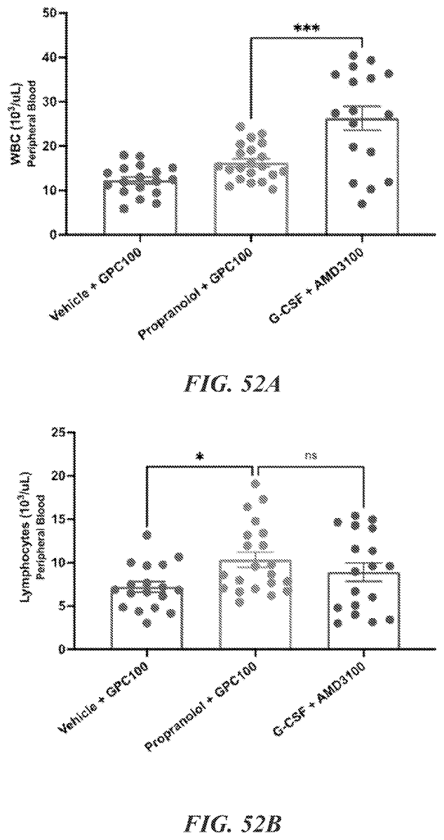

Data from 4 studies in which standard of care group was added are shown in FIGS. 52A-B. It was observed that standard of care regimen mobilized more WBCs compared to the propranolol and GPC100 combination. However, there was no difference in lymphocyte mobilization. Lymphocyte mobilization by propranolol and GPC100 combination treatment was comparable to G-CSF and AMD3100 combination treatment, suggesting the possibility to eliminate G-CSF for obtaining lymphocytes in the peripheral blood.

Study Design. The effect of propranolol on GPC100-induced mobilization with or without G-CSF was studied (Table 20).

TABLE 20
Triple combination dosing schedule

| | Day | Day | Day | Day | Day | Day | Day |
|---|---|---|---|---|---|---|---|
| Drug | 1 | 2 | 3 | 4 | 5 | 6 | 7 |
| Propranolol | + | + | + | + | + | + | + |
| 20 mg/kg IP | | | | | | | |
| G-CSF BID | | + | + | + | + | + | |
| 100 ug/kg/ | | | | | | | |
| injection SC | | | | | | | |
| GPC100 | | | | | | | + |
| 30 mg/kg IV | | | | | | | |

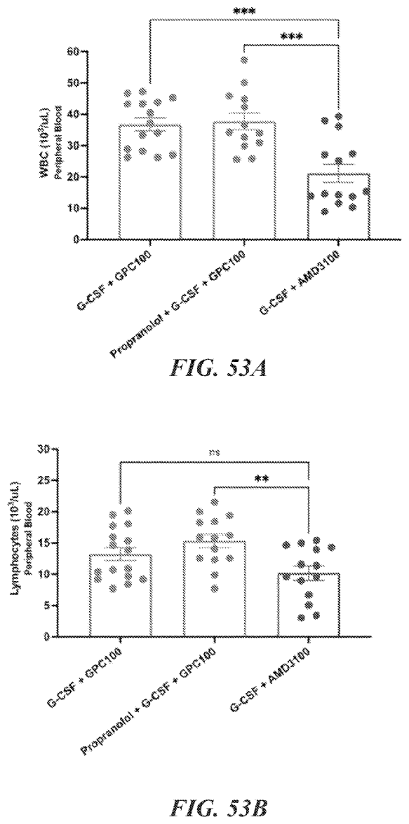

Data from 3 studies in which G-CSF combination group was added are shown in FIGS. 53A-B. It was observed that addition of propranolol to G-CSF and GPC100 combination mobilized significantly more WBC and lymphocytes compared to the standard of care for stem cell mobilization. When combined with G-CSF, GPC100 was observed to mobilize significantly more WBCs, compared to AMD3100. However, with addition of propranolol there was significantly more mobilization of lymphocytes.

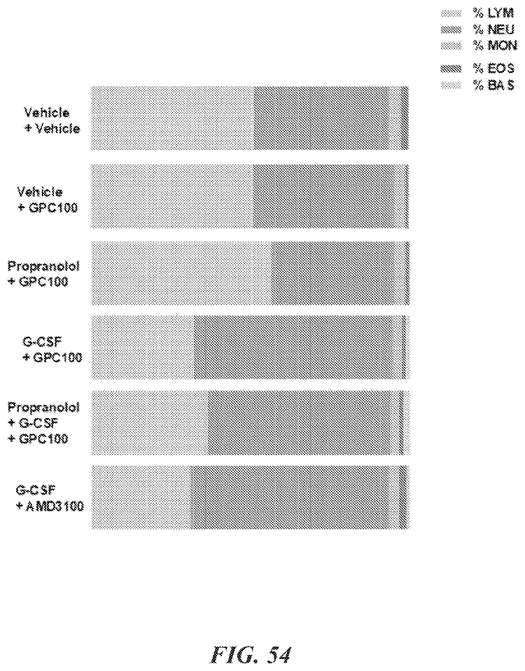

The distribution of WBC differentials is shown in FIG. 54. G-CSF was observed to mainly mobilize neutrophils. Furthermore, addition of propranolol to G-CSF slightly reduced neutrophil mobilization, while addition of propranolol to GPC100 slightly increased lymphocyte count in circulation.

### Example 9. Comparing Mobilization Between GPC100 and AMD3100

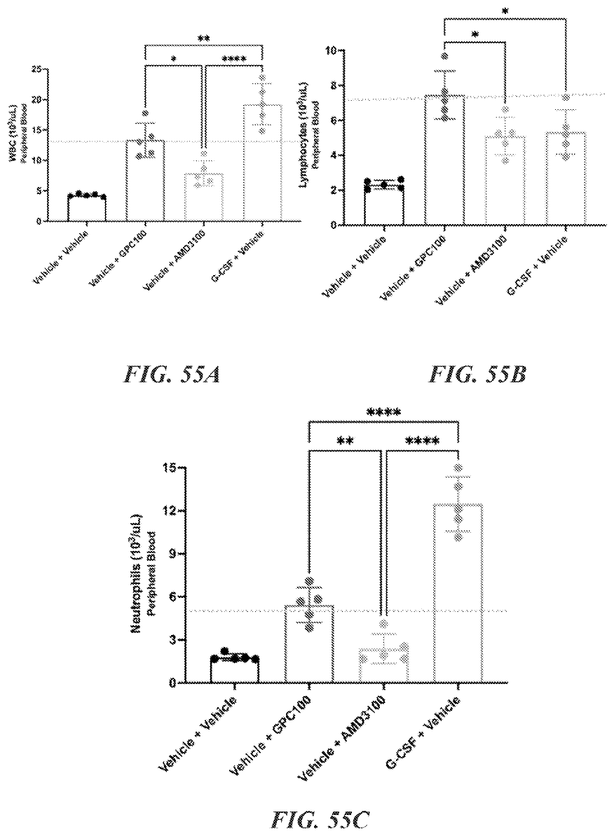

GPC100, AMD3100 or G-CSF induced WBC mobilization was studied (FIGS. 55A-C). Maximum mobilization was observed with G-CSF. GPC100 mobilized more lymphocytes than AMD3100 or G-CSF, while GPC100 mobilized more WBCs than AMD3100, and G-CSF mobilized more neutrophils than GPC100 or AMD3100.

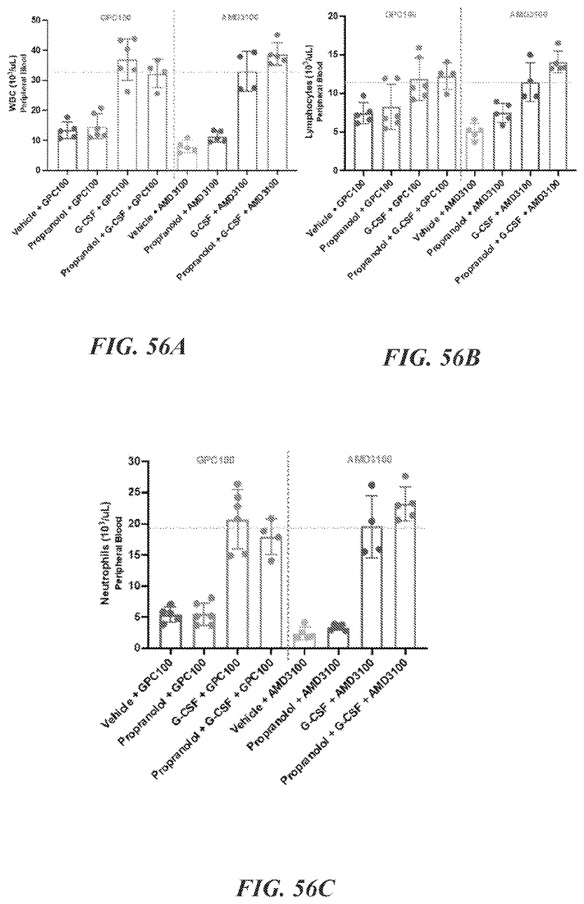

In a previous experiment, comparison between GPC100 and AMD3100 was performed (FIGS. 56A-C). It was the first study to show the effect of propranolol and triple combination with AMD3100. Propranolol was observed to slightly increase AMD3100-induced mobilization of lymphocytes.

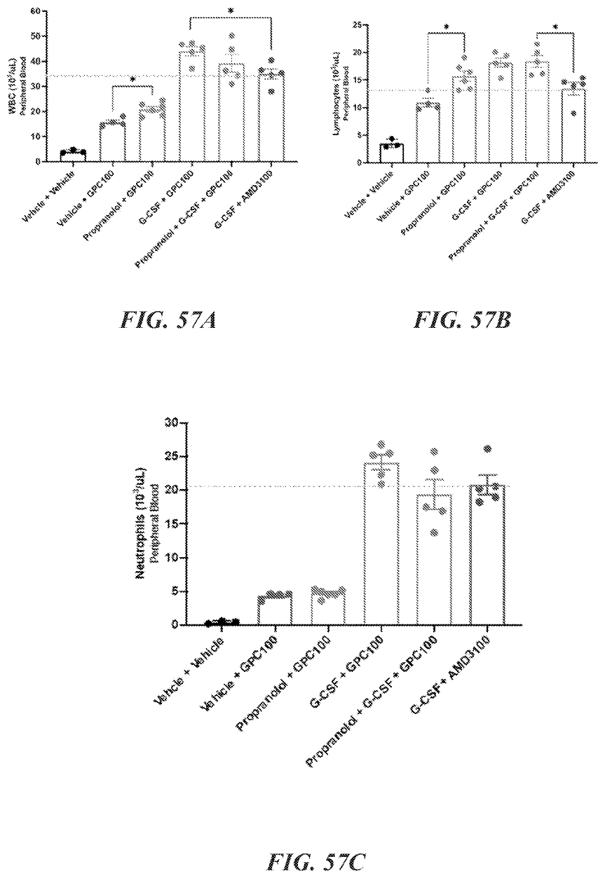

The effect of propranolol on GPC100-induced mobilization with or without G-CSF was studied, in comparison with standard of care (FIGS. 57A-C). When combined with G-CSF, GPC100 was observed to mobilize more WBCs compared to AMD3100. Propranolol and GPC100 combination resulted in increased circulating lymphocytes at levels similar to the standard of care (G-CSF+AMD3100).

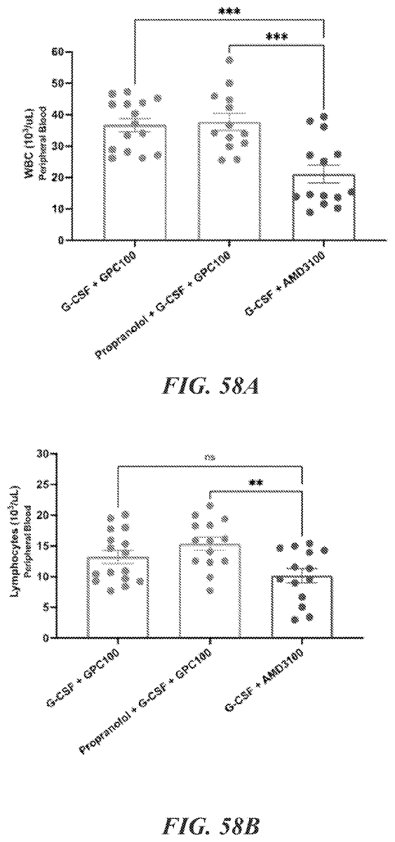

Data from 3 studies in which G-CSF combination group was added are shown in FIGS. 58A-B. When combined with G-CSF, GPC100 was observed to mobilize significantly more WBCs, compared to AMD3100. However, with addition of propranolol there was significantly more mobilization of lymphocytes.

### Example 10. GPC100 and Propranolol as Cell Mobilization Therapy for Autologous Stem Cell Transplant (ASCT)

Successful autologous stem cell transplant (ASCT) in multiple myeloma (MM) patients is often hindered by poor mobilization, with ˜1 in 7 patients failing to reach adequate number of CD34+ cells/kg. Small molecule inhibitors of CXCR4 like GPC100 and Plerixafor disrupt the CXCL12/CXCR4 axis critical for migration and retention of hematopoietic stem cells (HSC) in bone marrow. Here, we provide evidence that GPC100 in combination with Propranolol (Pro), a β2 adrenoceptor (B2AR) blocker (BB), and G-CSF, has the potential to be best-in-class mobilization therapy for ASCT.

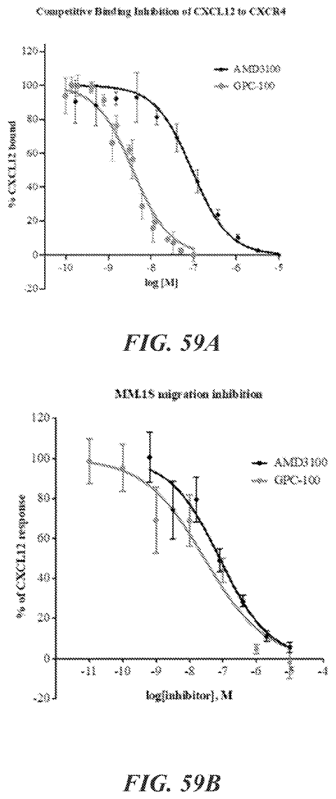

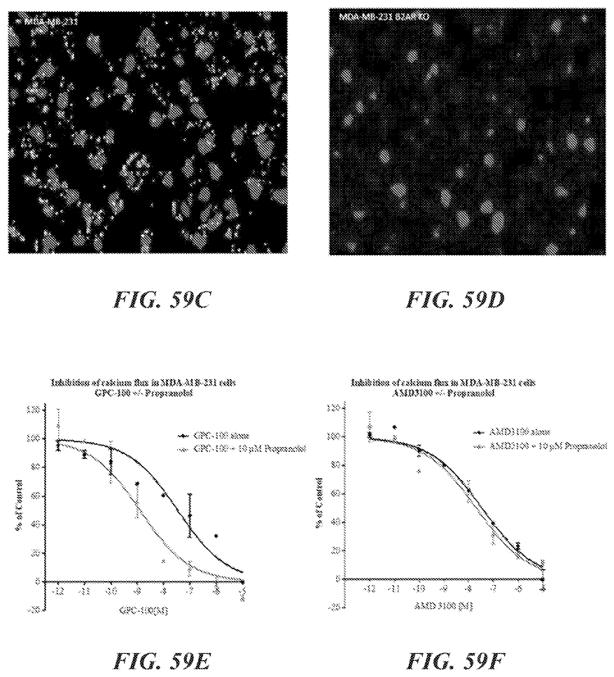

In vitro activity of GPC100 was investigated in cell-based assays (FIGS. 59A-59B). In the FRET ligand binding assay in HEK cells, GPC100 more potently inhibited binding of CXCL12 to CXCR4 than AMD3100 with a ˜30-fold better binding affinity (Ki of 1.6 vs 40 nM, respectively). Potent inhibition of CXCR4 was recapitulated in cell migration assays using a multiple myeloma cell line MM1.S, where GPC100 inhibited CXCL12-mediated migration with IC50 of 30 nM compared to the AMD3100 IC50 of 80 nM.

Previous studies indicate that stress hormones like epinephrine and norepinephrine exert stimulatory effects on cancer progression by modulating tumorigenesis, proliferation, and metastasis via B2AR signaling. In a recent study with 208 MM patients, overall survival was significantly longer in 37% of patients who reported BB usage for ≥3 months after diagnosis compared to those with no BB usage (107 vs 86 mos, Hwa et al., Eur J Haematol 2021). Furthermore, it has been demonstrated that BBs like Pro can shift bone marrow-derived cells to differentiate away from a myeloid bias to a phenotype consistent with CD34+ stem cells and genes associated with stem cells (Knight et al., Blood Adv 2020).

To investigate the interplay between CXCR4 and B2AR blockade in vitro, we performed interaction and functional studies (FIGS. 59C-59F). Using a proximity ligation assay (PLA) in the breast cancer cell line MDA-MB-231 endogenously expressing CXCR4 and B2AR, we detected CXCR4 and B2AR heteromers, while knock-out of B2AR expression led to a decrease in PLA signal, confirming the proximity of CXCR4 and B2AR. We also demonstrated a functional consequence of CXCR4 and B2AR using Ca2+ flux assays in MDA-MB-231 cells that demonstrated synergy when co-stimulating with CXCL12 and salmeterol, a B2AR agonist. Inhibition of Ca2+ flux by GPC100, and not AMD3100, is enhanced ˜30-fold by co-treatment with Pro (1.3 vs 30 nm). Taken together, our in vitro results suggested that GPC100 inhibition of CXCR4 can be modulated by Pro.

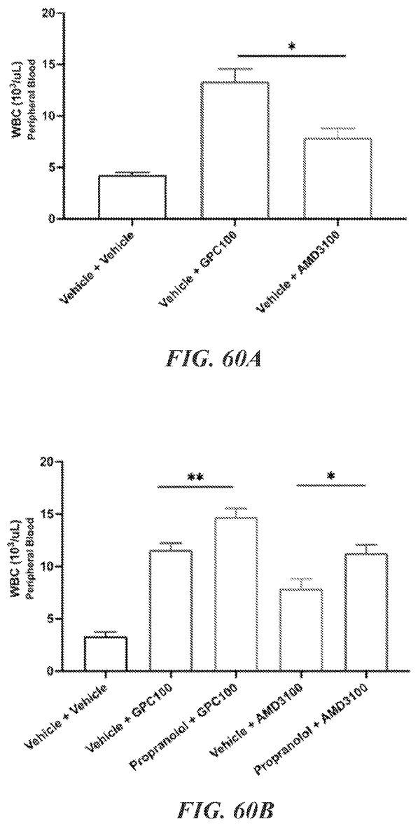

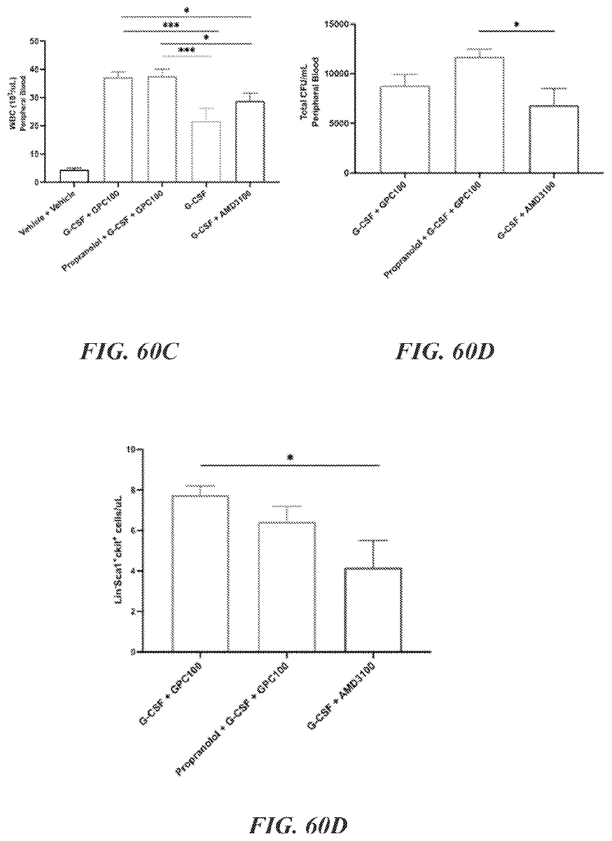

To obtain pre-clinical proof-of-concept, we determined the mobilization of white blood cells (WBCs) by a complete blood count (CBC) analysis, progenitor cells by a colony forming unit (CFU) assay, and HSC by flow cytometry, in C57/BL6 mice following GPC100 combination treatments. First, administration of GPC100 alone led to a greater WBC mobilization into the peripheral blood compared to AMD3100 alone (FIG. 60A). Next, mice were treated with Pro for 7 days followed by a single dose of GPC100 or AMD3100 on the 7th day (FIG. 60B). Our data demonstrated that combination treatment of GPC100 and Pro mobilized more WBCs compared to the combination of AMD3100 and Pro. Finally, we determined whether the triple combination of G-CSF+GPC100+Pro was beneficial over the current ASCT standards of care: G-CSF alone, or G-CSF combined with AMD3100 (FIG. 60C). We demonstrate that the triple combination leads to the highest mobilization of WBCs. In addition, we show that WBC mobilization is a predictor for mobilization of progenitor cells and stem cells, as we see a correlation between WBC counts and CFU indicative of progenitor cells (FIG. 60D) as well as the mobilized Lin−/sca−1+/c−kit+ (LSK) population indicative of mouse HSCs (FIG. 60E).

Our findings support the use of GPC100 and Pro, with or without G-CSF, for stem cell mobilization. This therapeutic strategy allows elimination of repeated daily injections of G-CSF, improving quality of life in patients, as well as providing a therapeutic option to patients that experience adverse effects from G-CSF. Additionally, treatment with G-CSF, GPC100 and Pro may prove to be best-in-class mobilization therapy for ASCT in MM patients, especially those that fail to mobilize with standard of care.

All publications and patent applications mentioned in this specification are herein incorporated by reference to the same extent as if each individual publication or patent application was specifically and individually indicated to be incorporated by reference.

While preferred embodiments have been shown and described herein, it will be obvious to those skilled in the art that such embodiments are provided by way of example only. It is intended that the following claims define the scope of the invention and that methods and structures within the scope of these claims and their equivalents be covered thereby.

### Example 11. GPC100 Induces WBC and Stem Cell Mobilization in Mice

Multiple myeloma (MM) is a leading hematological malignancy with an estimated 34,920 cases in the United States and approximately 588,161 cases worldwide each year (Cowan et al., 2022). Autologous Stem Cell Transplant (ASCT) is related to the overall management of eligible MM patients and has improved the anti-cancer response and survival compared to conventional chemotherapy (Devarakonda et al., 2021; Holsteain and McCarthy, 2016; Li and Zhu, 2019; Kumar et al., 2008). The success of ASCT relies in part on harvesting a sufficient number of hematopoietic stem cells (HSC), which are predominantly obtained by mobilizing the HSCs from bone marrow (BM) into the peripheral blood (PB) (Arora, Majhail, and Liu, 2019; Balassa, Danby, an dRocha, 2019). HSCs are phenotypically characterized by the expression of CD34. A minimum of around 2×10⁶ CD34⁺ cells/kg are used for HSC harvest, whereas the preferred numbers for improved engraftment and survival is >5-6×10⁶ CD34⁺ cells/kg (Toor et al., 2004; Tricot et al., 1995). Granulocyte-colony stimulating factor (G-CSF) is a clinical standard of care for HSC mobilization (DiPersio et al., 2009). However, G-CSF fails to mobilize optimal number of HSC in at least 40-50% MM patients (DiPersio et al., 2009; Demirer et al., 196). Some patients are treated with G-CSF in combination with a small molecule CXCR4 antagonist plerixafor (AMD3100) (DiPersio et al., 2009). Even with this combination treatment, 15-35% MM patients do not mobilize a sufficient number of cells (DiPersio et al., 2009). In a recent phase 3 clinical study, the combination of G-CSF and motixafortide, a peptide inhibitor of CXCR4, mobilized significantly greater CD34⁺ cells compared to G-CSF plus placebo (Crees et al., 2023). While this is promising, accumulating data suggests that MM therapies such as daratumumab or lenalidomide may negatively impact HSC mobilization (Hulin et al., 2021; Popat et al., 2021). Moreover, G-CSF is contraindicated in conditions like sickle cell disease for stem cell collection (Fitzhugh et al., 2009). These factors emphasize the unmet need for optimum HSC mobilization in MM patients, and also to expand ASCT across other disease indications (Pusic et al., 2008; Giralt et al., 2014).

CXCR4 is a member of the chemokine G protein-coupled receptor (GPCR) family and is expressed on HSCs (Wu et al., 2010; Mezzapelle et al., 2022; Guo et al., 2016). CXCR4 signaling, mediated by its natural ligand CXCL12, plays a role in cellular chemotaxis, as well as retention and survival of HSCs in BM (Guo et al., 2016). GPC-100, also known as Burixafor or TG-0054, is a novel small molecule antagonist of CXCR4 with a high binding affinity for CXCR4. GPC-100, in combination with G-CSF, has been tested clinically in MM patients as an HSC mobilizer (NCT02104427) (Schuster, 2021), and was shown to elicit a increase in HSCs with >5.0×10⁶ CD34⁺ cells/kg in 1-2 leukapheresis sessions (Setia et al., 2015). This result was comparable with the historical results from G-CSF plus AMD3100 treatment.

Previous studies suggest that CXCR4 physically interacts with the beta-2-adrenergic receptor or B2AR (gene ADRB2) in cells that ectopically overexpress both receptors (Nakai et al., 2014; LaRocca et al., 2010; Nakai, Leach, and Suzuki, 2021). In lymph nodes, the CXCR4-B2AR complex was thought to enhance lymphocyte retention by CXCR4 and inhibit their mobilization (Nakai et al., 2014). β₂AR is also expressed on HSCs and the adrenergic signaling plays a role in regulating HSC niche in BM (Spiegel et al., 2008; Saba et al., 2015; Maestroni, 2020; Katayama et al., 2006). The natural ligands of β₂AR, epinephrine and norepinephrine, influence the turnover, trafficking, and were shown to reduce the proliferative and differentiation capacity of the HSCs (Hanoun et al., 2015; Schraml et al., 2009). When human HSCs were co-stimulated with G-CSF and β₂AR agonists, the expression of CXCR4 on HSCs increased, suggesting that the interactions between β₂AR agonists and G-CSF in BM niche promote HSC retention by CXCR4 and impair mobilization by G-CSF (Saba et al., 2015).

Studies have noted the link between beta adrenergic inhibitor (beta blockers) usage and positive survival outcome in several cancer types including MM (Hwa et al., 2017; Hwa et al., 2021). The MM microenvironment is known to cause dysregulation of HSC function leading to changes in gene expression and altered hematopoietic differentiation (Bruns et al., 2012; Knight et al., 2020). A Phase II biomarker-driven randomized study showed that in MM patients, the FDA-approved non-selective beta blocker propranolol shifted cell differentiation away from the myeloid-lineage bias to an upregulation of CD34⁺ cells and enhanced engraftment (Knight et al., 2020). Furthermore, propranolol demonstrated the ability to inhibit the BM sympathetic nervous system-induced shift from basal gene expression profile to a more inflammatory gene expression pattern termed as Conserved Transcriptional Response to Adversity (CTRA), which is associated with poor outcomes in ASCT (Knight et al., 2020). In another study, BM samples from MM patients showed that propranolol can augment differentiation of HSCs into megakaryocyte-erythrocyte progenitors and reduce the number of granulocyte-monocyte progenitor cells, which are known to contribute to a pro-tumorigenic niche (Nair et al., 2022). Therefore, considering the positive effect of propranolol on HSC proliferation and differentiation, as well as possible crosstalk between β₂AR and CXCR4 in BM, co-inhibition of the two pathways may improve HSC mobilization.

In the present study, in vivo mobilization efficacy of GPC-100 in comparison with AMD3100 is reported. Furthermore, the report demonstrates enhanced mobilization in vivo by GPC-100 in combination with propranolol and propose a new strategy for clinical application in stem cell mobilization.

### Methods:

### In Vivo Mobilization:

C57BL/6J or Balb/c mice (female, 6-9 weeks old) were randomized for each study so that all treatment groups contained similar age and weight distributions. Studies were performed at a facility accredited by the Association for Assessment and Accreditation of Laboratory Animal Care International and Institutional Animal Care and Use Committee. PB was collected via cardiac puncture on the 7th day 2 h after GPC-100 and 1 h after AMD3100 administration. Blood samples were processed for complete blood count (CBC) analysis using the Abaxis VetScan HM5 hematology analyzer.

TABLE 21
Dosing for in vivo mobilization

| | | | | | Dosing |
|---|---|---|---|---|---|
| Treatment | Dose | | Route | Volume | Frequency |
| GPC-100 | 30 | mg/kg | Intravenous | 5 mL/kg | Single injection |
| AMD3100 | 5 | mg/kg | subcutaneous | 10 mL/kg | Single injection |
| Propranolol | 20 | mg/kg | Intraperitoneal | 10 mL/kg | Once a day |
| | | | | | (QD) × 7 days |
| G-CSF | 0.1 | mg/kg | subcutaneous | 10 mL/kg | Twice a day |
| | | | | | (BID) × 5 days |

GPC-100 and AMD3100 were administered alone or co-administered with propranolol on day 7. In the triple combination group, G-CSF was administered from days 2 to 6. CXCR4 antagonists and propranolol were injected 12 hours later. All compounds were reconstituted in PBS. Control mice received PBS in the same volume.

### Colony Forming Unit (CFU) Assay:

8×10⁵ mononuclear cells isolated from PB post-CBC analysis were added to tubes of semisolid methylcellulose medium (StemCell Technologies) known to support erythroid and myeloid progenitors (Kronstein-Wiedemann, 2019). Seven days later, colonies showing appearance of granulocyte-monocyte progenitors (CFU-GM) and burst forming erythroid units (BFU-E) formed and were counted by a blinded experimenter. Total CFU were calculated as a total number of CFU-GM and BFU-U colonies.

### Flow Cytometry:

To determine the mobilization of mouse HSC, characterized as LSK cells (Lineage−Sca−1+c−Kit+) (Challen et al., 2009), mononuclear cells isolated from PB post-CBC analysis were stained with anti-lineage cocktail, c-Kit and Sca−1 antibodies. Samples were acquired with a Cytek Aurora spectral flow cytometer (Fremont, CA) and data was analyzed with CellEngine software. Gating was determined using FMO controls. The percentage of C-Kit⁺ Sca−1⁺ cells as a subset of parent Lin-cells were used to determine the total number of LSK cells/uL of blood.

TABLE 22
Antibodies used for flow cytometry

| Target | Clone | Fluor | Supplier |
|---|---|---|---|
| Mouse Lineage | 500A2, M1/70, | V450 | BD Biosciences |
| Antibody Cocktail | RA3-6B2, TER-119, | | 561301 |
| | RB6-8C5 | | |
| CD117 (c-Kit) | 2B8 | Alexa Fluor 647 | Biolegend 105818 |
| Ly6A/E | D7 | Brilliant Violet 711 | Biolegend 108131 |
| CD48 | HM48-1 | Brilliant Violet 510 | Biolegend 103443 |
| CD150 (SLAM) | TC15-12F12.2 | PE/Cyanine7 | Biolegend 115914 |
| Viability | N/A | eFluor 780 | Invitrogen 65-0865-18 |

Statistical analyses: Data analyses were performed using GraphPad Prism and all data are presented as mean±SEM. Comparisons of data across dosing conditions were made using the Mann-Whitney test or one-way ANOVA. P<0.05 was considered statistically significant for all tests.

### Experimental Design:

First, mobilization of WBC and LSK stem cells by GPC-100 was determined following single IV administration. For the subsequent studies, WBC mobilization was used as a marker for stem cell mobilization. To identify the dose of propranolol to use in combination with GPC-100, propranolol was administered IP at 5, 10, 20, and 40 mg/kg for 7 days, followed by co-administration of GPC-100 on the 7th day. Propranolol was administered at 20 mg/kg since this dose significantly improved GPC-100 induced mobilization. Mobilization of LSK stem cells was determined by flow for the propranolol and GPC-100 combination. Next, combination of GPC-100 and propranolol was compared with G-CSF alone for WBC mobilization. For this study, G-CSF was administered for 5 days, two times daily. Lastly, a triple combination with G-CSF, GPC-100 and propranolol was investigated in comparison with G-CSF plus AMD3100 for WBC and stem cell mobilization in a phenotypic analysis and colony forming unit assay. For all studies, blood was collected 2 hours after GPC-100, 1 hour after AMD3100 and 12 hours after G-CSF.

### Results

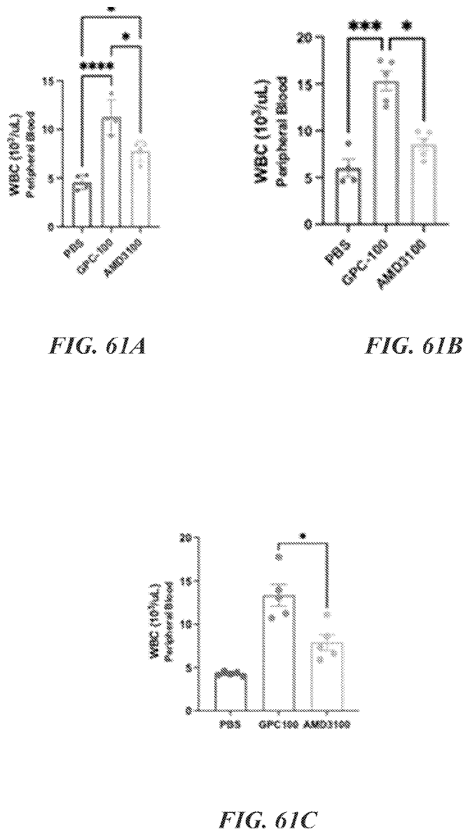

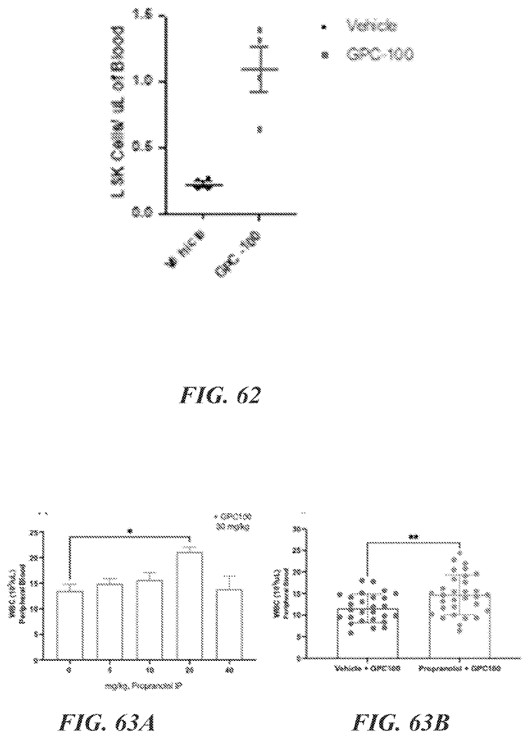

A single administration of GPC-100 (30 mg/kg, IV) induced WBC mobilization in PB that peaked at 2 hours. Numerous studies in mice report the peak mobilization by AMD3100 (5 mg/kg, SC) at 1 hour (e.g., Broxmeyer et al. 2005). Therefore, PB WBC counts post-GPC-100 and post-AMD3100 were determined at time-points and doses where maximum mobilization is observed for each antagonist. GPC-100 elicited increase in PB WBC count in both C57/BL6 and balb/c mouse strains (FIG. 1A). When compared to AMD3100 in 3 separate studies (FIGS. 61A,61B,61C), GPC-100 produced a 2-3-fold increase, whereas AMD3100 produced <2-fold increase in WBCs compared to the vehicle. The increase in WBCs by both antagonists included increases in lymphocytes and neutrophils. No changes in the platelet count, hemoglobin or other red blood cell parameters were observed. Determination of LSK cells by flow cytometry indicated that GPC-100 also mobilized hematopoietic stem cells (FIG. 62).

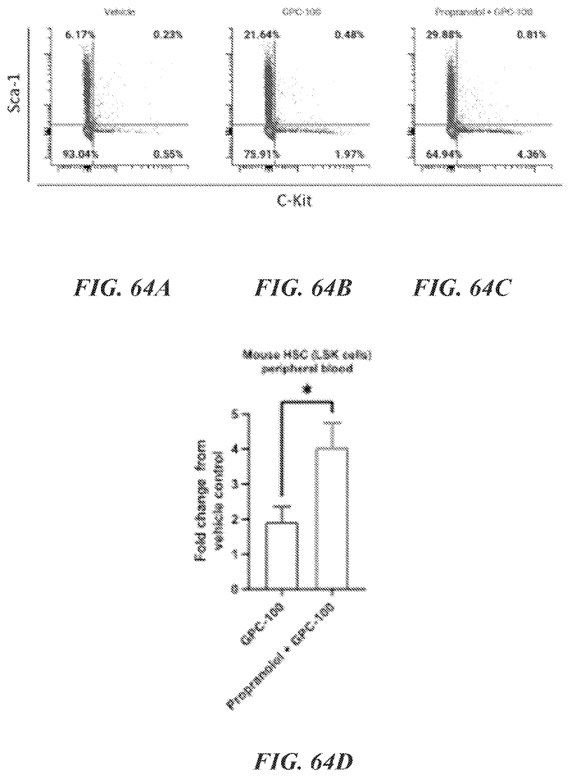

To evaluate the impact of β₂AR blockade in vivo, mice were administered propranolol. Propranolol dose was selected based on the dose titration (5-40 mg/kg, IP) when combined with GPC-100 (FIG. 63A). Pretreatment with propranolol (20 mg/kg, IP) over 7 days significantly improved GPC-100 induced mobilization (FIG. 63B). Phenotypic analyses for LSK cells by flow cytometry also indicated that LSK cell mobilization by GPC-100 was enhanced by propranolol (FIGS. 64A-64D).

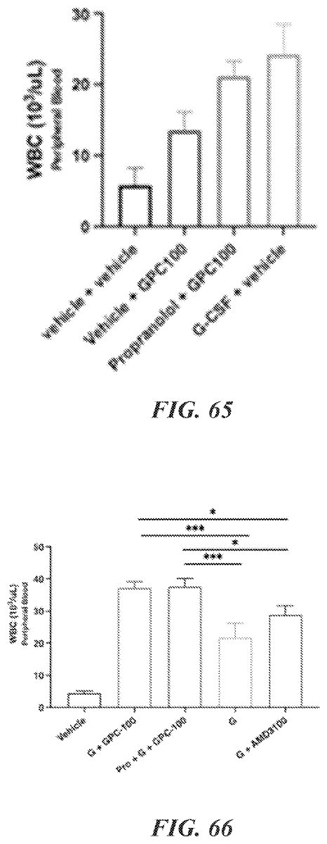

Next, mobilization by GPC-100 and propranolol combination was compared with the standard of care, G-CSF. Propranolol induced 4.1-fold, whereas G-CSF induced a comparable increase of 4.5-fold in mobilizing the WBCs (FIG. 65).

Triple combination of G-CSF, GPC-100 and propranolol was compared with the current ASCT standards of care, i.e., G-CSF alone or in combination with AMD3100. The triple combination as well as the combination of G-CSF and GPC-100 induced an 8.2- and 8.4-fold increase in WBC mobilization, respectively, that was significantly greater compared to the increased WBC count by G-CSF alone (4.5-fold) or G-CSF plus AMD3100 (6.6-fold) (FIG. 66).

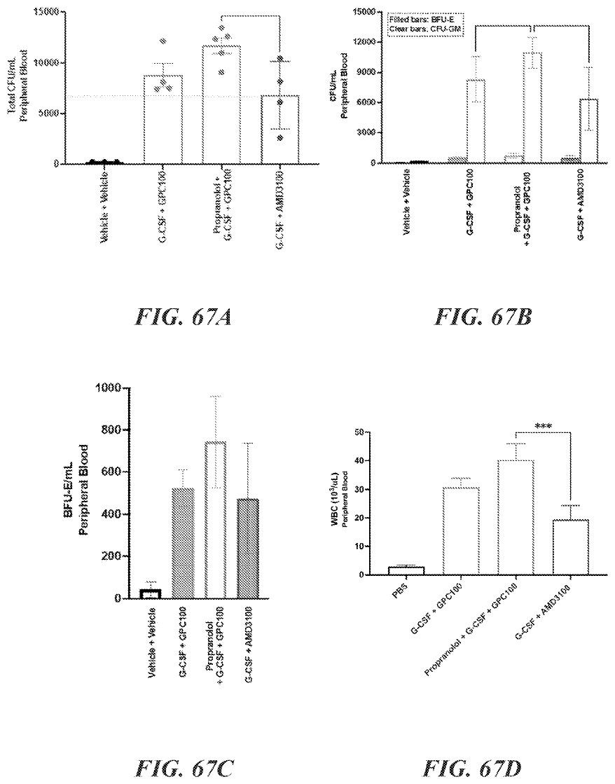

Further experiments were conducted to determine if increased WBC count in circulation reflected hematopoietic stem and progenitor cell (HSPC) mobilization. CFU assay was conducted to measure the mobilized HSPCs based on their ability to form CFU-GM and BFU-E colonies. The triple combination produced a 47-fold increase in CFUs over vehicle control compared to a 35-fold and 27-fold increase over vehicle from G-CSF plus GPC-100 and G-CSF plus AMD3100 treatments, respectively (FIG. 67A-67D).

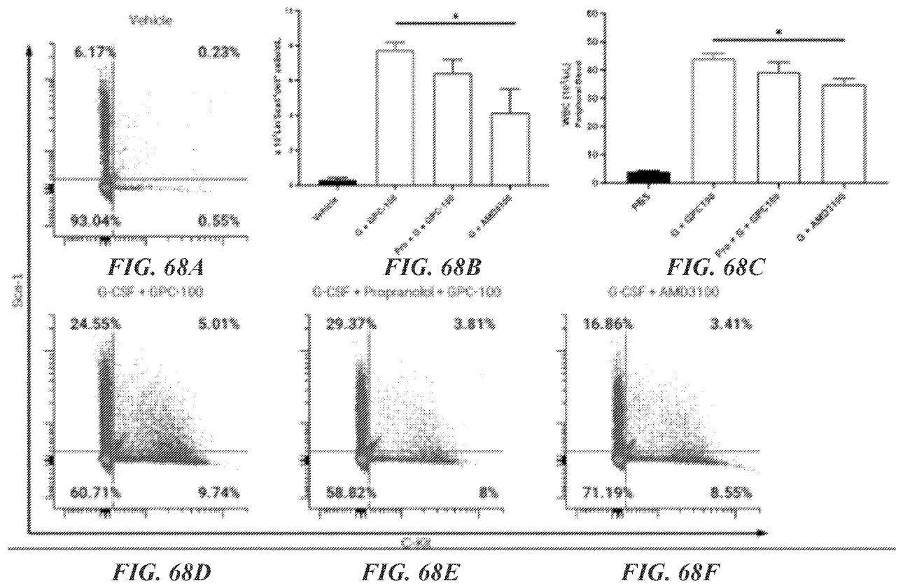

Phenotypic analysis showed that G-CSF plus AMD3100 treatment resulted in a 13-fold increase in LSK cells in PB compared to the vehicle. In comparison, G-CSF and GPC-100 combination with and without propranolol resulted in 20-fold and 24-fold increase in LSK cells, respectively (FIG. 68A-68F). The pattern of LSK and CFU counts across the different drug combinations was consistent with the WBC count from matched samples (FIG. 67A-67D & 68A-68F), supporting the use of WBC counts as a surrogate marker for stem cell mobilization.

The present studies show that GPC-100 is a potent hematopoietic mobilizer, and its mobilizing effect is enhanced by propranolol. The studies also also show that GPC-100-induced increase in the mobilization by G-CSF is superior to the combination of G-CSF and AMD3100. Addition of propranolol to G-CSF and GPC-100 mobilized significantly more hematopoietic stem cells capable of differentiating into multipotent progenitors. The data as well as the previous reports have shown that HSPC mobilization is associated with a concomitant increase in circulating WBCs (Vater et al., 2013; Almeida-Neto et al., 2020; Abraham et al., 2007; Lee et al., 2014). Effects of propranolol seen in this study could be explained by the independent effect of propranolol on HSPCs or the interactions between β₂AR and CXCR4.

The present studies also show that propranolol and GPC-100 combination showed a 4-fold increase, whereas G-CSF induced a 4.5-fold increase in WBCs over the vehicle control. This observation is important since it suggests the possibility of comparable HSC mobilization without the use of G-CSF. Elimination of G-CSF from the treatment may reduce the risk of moderate to severe side effects of G-CSF such as severe bone pain and rarely, splenic rupture.

Addition of propranolol to G-CSF and GPC-100 (triple combination) increased PB CFU count that was significantly greater than G-CSF plus AMD3100. This indicates that the triple combination mobilized a higher number of viable cells that were functionally capable of differentiating into myeloid and erythroid multipotent progenitors. Moreover, phenotypic analysis revealed more LSK cells in PB with G-CSF and GPC-100 treatment with or without propranolol compared to G-CSF plus AMD3100. The present study was performed in naïve mice and the effects of propranolol will likely be amplified in a model wherein the BM microenvironment and HSC differentiation is compromised (Giles et al., 2016).

In summary, our preclinical findings support the addition of propranolol to GPC-100-induced stem cell mobilization for ASCT in MM patients. The triple combination of GPC-100, propranolol, and G-CSF can potentially be best-in-class and target patient populations where other mobilization regimens have failed. Propranolol could prove to be a safe, accessible, and inexpensive option to supplement the mobilization therapies for greater stem cell yields in fewer apheresis sessions and reduce the financial burden on patients and healthcare systems. The relevant clinical study is registered as a two-arm Phase 2 clinical trial (NCT05561751) with a GPC-100 plus propranolol arm and GPC-100, propranolol, and G-CSF arm.

### EXEMPLARY EMBODIMENTS

In an embodiment, disclosed herein is a method of mobilizing a cell in a subject, the method comprising: blocking CXCR4 signaling and beta-adrenergic receptor signaling in the subject.

In an embodiment, disclosed herein is a method of inducing cell mobilization in a subject, the method comprising: blocking CXCR4 signaling and beta-adrenergic receptor signaling in the subject.

In an embodiment, disclosed herein is a method of enhancing apheresis in a subject, the method comprising: blocking CXCR4 signaling and beta-adrenergic receptor signaling in the subject.

In an embodiment, disclosed herein is a method of enhancing apheresis by inducing cell mobilization in a subject, the method comprising: blocking CXCR4 signaling and beta-adrenergic receptor signaling in the subject.

In an embodiment, disclosed herein is a method of enhancing apheresis by mobilizing a cell in a subject, the method comprising: blocking CXCR4 signaling and beta-adrenergic receptor signaling in the subject.

In an embodiment, the blocking beta-adrenergic receptor signaling is performed before the blocking CXCR4 signaling.

In an embodiment, the blocking beta-adrenergic receptor signaling continues after the blocking CXCR4 signaling is terminated.

In an embodiment, the blocking CXCR4 signaling comprises administering a CXCR4 inhibitor to the subject.

In an embodiment, the blocking beta-adrenergic receptor signaling comprises administering a beta-adrenergic receptor inhibitor to the subject.

In an embodiment, the cell is a stem cell.

In an embodiment, the blocking CXCR4 signaling comprises administering a CXCR4 inhibitor to the subject and the blocking beta-adrenergic receptor signaling comprises administering a beta-adrenergic receptor inhibitor to the subject.

In an embodiment, the cell is a stem cell.

In an embodiment, disclosed herein is a method of mobilizing a stem cell in a subject, the method comprising: administering a beta-adrenergic receptor inhibitor and a CXCR4 inhibitor to the subject.

In an embodiment, disclosed herein is a method of inducing stem cell mobilization in a subject, the method comprising: administering a beta-adrenergic receptor inhibitor and a CXCR4 inhibitor to the subject.

In an embodiment, disclosed herein is a method of enhancing apheresis in a subject, the method comprising: administering a beta-adrenergic receptor inhibitor and a CXCR4 inhibitor to the subject.

In an embodiment, disclosed herein is a method of enhancing apheresis by inducing cell mobilization in a subject, the method comprising: administering a beta-adrenergic receptor inhibitor and a CXCR4 inhibitor to the subject.

In an embodiment, disclosed herein is a method of enhancing apheresis by mobilizing a cell in a subject, the method comprising: administering a beta-adrenergic receptor inhibitor and a CXCR4 inhibitor to the subject.

In an embodiment, the administering the beta-adrenergic receptor inhibitor is performed before the administering the CXCR4 inhibitor.

In an embodiment, the administering the beta-adrenergic receptor inhibitor continues after the administering the CXCR4 inhibitor is terminated.

In an embodiment, the method further comprises administering G-CSF to the subject.

In an embodiment, the administering the beta-adrenergic receptor inhibitor and the CXCR4 inhibitor to the subject is performed in the absence of G-CSF.

In an embodiment, disclosed herein is a method of mobilizing a stem cell in a subject, the method comprising: administering a CXCR4 inhibitor and G-CSF to the subject, in the absence of a beta-adrenergic receptor inhibitor.

In an embodiment, disclosed herein is a method of inducing stem cell mobilization in a subject, the method comprising: administering a CXCR4 inhibitor and G-CSF to the subject, in the absence of a beta-adrenergic receptor inhibitor.

In an embodiment, disclosed herein is a method of enhancing apheresis in a subject, the method comprising: administering a CXCR4 inhibitor and G-CSF to the subject, in the absence of a beta-adrenergic receptor inhibitor.

In an embodiment, disclosed herein is a method of enhancing apheresis by inducing cell mobilization in a subject, the method comprising: administering a CXCR4 inhibitor and G-CSF to the subject, in the absence of a beta-adrenergic receptor inhibitor.

In an embodiment, disclosed herein is a method of enhancing apheresis by mobilizing a cell in a subject, the method comprising: administering a CXCR4 inhibitor and G-CSF to the subject, in the absence of a beta-adrenergic receptor inhibitor.

In an embodiment, the beta-adrenergic receptor inhibitor is an ADRB2 inhibitor.

In an embodiment, the beta-adrenergic receptor inhibitor is selected from the group consisting of alprenolol, atenolol, betaxolol, bupranolol, butoxamine, carazolol, carvedilol, CGP 12177, cicloprolol, ICI 118551, ICYP, labetalol, levobetaxolol, levobunolol, LK 204-545, metoprolol, nadolol, NIHP, NIP, propafenone, propranolol, sotalol, SR59230A, and timolol.

In an embodiment, the beta-adrenergic receptor inhibitor is selected from the group consisting of propranolol, nadolol, and ICI 118551.

In an embodiment, the beta-adrenergic receptor inhibitor is propranolol.

In an embodiment, the CXCR4 inhibitor is selected from the group consisting of ALX40-4C, AMD070 (AMD11070, X4P-001), AMD3100 (plerixafor), AMD3465, ATI 2341, BKT140 (BL-8040; TF14016; 4F-Benzoyl-TN14003), CTCE-9908, CX549, D-[Lys3] GHRP-6, FC122, FC131, GMI-1359, GSK812397, GST-NT21MP, isothiourea-la, isothiourea-1t (IT1t), KRH-1636, KRH-3955, LY2510924, MSX-122, N-[11C] Methyl-AMD3465, POL6326, SDF-1 1-9 [P2G] dimer, SDF1 P2G, T134, T140, T22, TC 14012, TG-0054 (Burixafor), USL311, viral macrophage inflammatory protein-II (vMIP-II), WZ811, [64Cu]-AMD3100, [64Cu]-AMD3465, [68Ga] pentixafor, [90Y] pentixather, [99mTc] 02-AMD3100, [177Lu] pentixather, and 508MCI (Compound 26).

In an embodiment, the CXCR4 inhibitor is selected from the group consisting of AD-214, AMD070 (AMD11070, X4P-001), AMD3100 (plerixafor), BKT140 (BL-8040; TF14016; 4F-Benzoyl-TN14003), CTCE-9908, LY2510924, LY2624587, T140, TG-0054 (Burixafor), PF-06747143, POL6326, and ulocuplumab (MDX1338/BMS-936564).

In an embodiment, the CXCR4 inhibitor is TG-0054 (burixafor).

In an embodiment, the CXCR4 inhibitor is AMD3100 (plerixafor).

In an embodiment, the CXCR4 inhibitor is ulocuplumab (MDX1338/BMS-936564).

In an embodiment, the administering the CXCR4 inhibitor to the subject comprises administering TG-0054 (burixafor) and propranolol.

In an embodiment, the administering the CXCR4 inhibitor to the subject comprises administering AMD3100 (plerixafor) and propranolol.

In an embodiment, the administering the CXCR4 inhibitor to the subject comprises administering ulocuplumab (MDX1338/BMS-936564) and propranolol.

In an embodiment, the administering a combination of the CXCR4 inhibitor and the G-CSF induces an enhanced amount of cell mobilization relative to the amount of cell mobilization induced by the CXCR4 inhibitor only.

In an embodiment, the administering a combination of the CXCR4 inhibitor and the G-CSF mobilizes a cell by an amount enhanced relative to the amount of cell mobilization induced by the CXCR4 inhibitor only.

In an embodiment, the administering a combination of the CXCR4 inhibitor and the G-CSF induces an enhanced amount of apheresis relative to the amount of apheresis induced by the CXCR4 inhibitor only.

In an embodiment, the administering a combination of the CXCR4 inhibitor and the beta-adrenergic receptor inhibitor induces an enhanced amount of cell mobilization relative to the amount of cell mobilization induced by the CXCR4 inhibitor only.

In an embodiment, the administering a combination of the CXCR4 inhibitor and the beta-adrenergic receptor inhibitor mobilizes a cell by an amount enhanced relative to the amount of cell mobilization induced by the CXCR4 inhibitor only.

In an embodiment, the administering a combination of the CXCR4 inhibitor and the beta-adrenergic receptor inhibitor induces an enhanced amount of apheresis relative to the amount of apheresis induced by the CXCR4 inhibitor only.

In an embodiment, the administering a combination of the CXCR4 inhibitor, the beta-adrenergic receptor inhibitor, and the G-CSF induces an enhanced amount of cell mobilization relative to the amount of cell mobilization induced by the CXCR4 inhibitor and the beta-adrenergic receptor inhibitor only.

In an embodiment, the administering a combination of the CXCR4 inhibitor, the beta-adrenergic receptor inhibitor, and the G-CSF mobilizes a cell by an amount enhanced relative to the amount of cell mobilization induced by the CXCR4 inhibitor and the beta-adrenergic receptor inhibitor only.

In an embodiment, the administering a combination of the CXCR4 inhibitor and the beta-adrenergic receptor inhibitor, and the G-CSF induces an enhanced amount of apheresis relative to the amount of apheresis induced by the CXCR4 inhibitor and the beta-adrenergic receptor inhibitor only.

In an embodiment, the administering a combination of TG-0054 (burixafor) and the G-CSF induces an enhanced amount of cell mobilization relative to the amount of cell mobilization induced by AMD3100 (plerixafor) and the G-CSF.

In an embodiment, the administering a combination of the TG-0054 (burixafor) and the G-CSF mobilizes a cell by an amount enhanced relative to the amount of cell mobilization induced by the AMD3100 (plerixafor) and the G-CSF.

In an embodiment, the administering a combination of the TG-0054 (burixafor) and the G-CSF induces an enhanced amount of apheresis relative to the amount of apheresis induced by the AMD3100 (plerixafor) and the G-CSF.

In an embodiment, an enhanced amount of cell mobilization or apheresis is measured by a method selected from the group consisting of complete blood count (CBC) analysis, flow cytometry, and colony forming unit (CFU) assay.

In an embodiment, the enhanced amount of cell mobilization or apheresis is measured by flow cytometry.

In an embodiment, the flow cytometry is performed on (Lin−Sca1+c−Kit+) LSK cells.

In an embodiment, the enhanced amount of cell mobilization or apheresis is measured by colony forming unit (CFU) assay.

In an embodiment, the subject has a CXCR4 protomer in the cell.

In an embodiment, the subject has an ADRB2 protomer in the cell.

In an embodiment, the subject has a CXCR4 protomer and an ADRB2 protomer in the cell.

In an embodiment, the subject has a CXCR4-ADRB2 heteromer in the cell.

In an embodiment, i) the CXCR4-ADRB2 heteromer has an enhanced amount of downstream calcium mobilization relative to downstream calcium mobilization from a CXCR4 protomer or ADRB2 protomer; and ii) the administered combination of inhibitors suppresses the enhanced downstream calcium mobilization from said CXCR4-ADRB2 heteromer in the stem cell. In an embodiment, the cell is a stem cell.

In an embodiment, the stem cell is selected from the group consisting of a hematopoietic stem cell, a hematopoietic progenitor cell, a mesenchymal stem cell, an endothelial progenitor cell, a neural stem cell, an epithelial stem cell, a skin stem cell, and a cancer stem cell.

In an embodiment, the stem cell is a hematopoietic stem cell or a hematopoietic progenitor cell.

In an embodiment, the hematopoietic stem cell or the hematopoietic progenitor cell is mobilized from bone marrow to peripheral blood.

In an embodiment, the mobilized hematopoietic stem cell or hematopoietic progenitor cell is collected for transplantation to a patient having cancer.

In an embodiment, the cancer is selected from the group consisting of lymphoma, leukemia, and myeloma.

In an embodiment, the cancer is non-Hodgkin lymphoma (NHL), acute myeloid leukemia (AML), acute lymphoblastic leukemia (ALL), or multiple myeloma (MM).

In an embodiment, the stem cell is a mesenchymal stem cell.

In an embodiment, the mesenchymal stem cell is mobilized from bone marrow to peripheral blood.

In an embodiment, the mesenchymal stem cell is mobilized for treatment of a condition selected from the group consisting of neurological disorder, cardiac ischemia, myocardial infarction, diabetes, tissue repair, bone and cartilage disease, autoimmune disease, graft versus host disease, Crohn's disease, multiple sclerosis, systemic lupus erythematosus, and systemic sclerosis.

In an embodiment, the stem cell is a cancer stem cell.

In an embodiment, the cancer stem cell is mobilized into blood.

In an embodiment, the cancer stem cell is mobilized for treatment of a cancer.

In an embodiment, the cell is an immune cell.

In an embodiment, the immune cell is a white blood cell.

In an embodiment, the white blood cell is a lymphocyte.

In an embodiment, the lymphocyte is selected from the group consisting of a T cell, a B cell, and a natural killer (NK) cell.

In an embodiment, the lymphocyte is a T cell.

In an embodiment, the lymphocyte is a natural killer (NK) cell.

In an embodiment, the white blood cell is a granulocyte.

In an embodiment, the granulocyte is selected from the group consisting of a neutrophile, an eosinophile, and a basophile.

In an embodiment, the granulocyte is a neutrophile.

In an embodiment, the white blood cell is a monocyte.

In an embodiment, the immune cell is mobilized from bone marrow to peripheral blood.

In an embodiment, the immune cell is mobilized from lymph node to peripheral blood.

In an embodiment, the mobilized immune cell is used for adoptive cell therapy (ACT).

In an embodiment, the adoptive cell therapy (ACT) is chimeric antigen receptor (CAR) T cell therapy.

In an embodiment, the adoptive cell therapy (ACT) is natural killer (NK) cell therapy.

In an embodiment, the adoptive cell therapy (ACT) is engineered T-cell receptor (TCR) therapy.

In an embodiment, the adoptive cell therapy (ACT) is tumor-infiltrating lymphocyte (TIL) therapy.

1-89. (canceled)

90. A method for treatment of cancer comprising administering, alone or in combination, a CXCR4 inhibitor and a beta-2-adrenergic receptor (ADRB2) inhibitor to a subject, wherein the treatment comprises stem cell transplantation.

91. The method of claim 90, wherein the administering comprises administering the ADRB2 inhibitor and the CXCR4 inhibitor simultaneously, concurrently, or sequentially to the subject.

92. The method of claim 90, wherein the administering comprises administering the ADRB2 inhibitor to the subject before administering the CXCR4 inhibitor to the subject.

93. The method of claim 90, wherein administering the ADRB2 inhibitor is initiated at a first specific time interval before administering the CXCR4 inhibitor, and wherein the first specific time interval is 6 days to 7 days.

94. The method of claim 90, wherein the treatment comprises hematopoietic stem cell transplantation or hematopoietic progenitor cell transplantation.

95. The method of claim 90, wherein the stem cell transplantation comprises autologous stem cell transplantation.

96. The method of claim 90, wherein the stem cell transplantation comprises allogeneic stem cell transplantation.

97. The method of claim 90, wherein the cancer is selected from lymphoma, leukemia, and myeloma.

98. The method of claim 90, wherein the cancer is selected from non-Hodgkin lymphoma, acute myeloid leukemia, acute lymphoblastic leukemia, and multiple myeloma.

99. The method of claim 90, wherein the cancer is multiple myeloma.

100. The method of claim 90, wherein the CXCR4 inhibitor is selected from the group consisting of ALX40-4C, AMD070, plerixafor, AMD3465, ATI 2341, BKT140, CTCE-9908, CX549, D-[Lys3] GHRP-6, FC122, FC131, GMI-1359, GSK812397, GST-NT21MP, isothiourea-la, isothiourea-It (IT1t), KRH-1636, KRH-3955, LY2510924, MSX-122, N-[11C] Methyl-AMD3465, POL6326, SDF-1 1-9 [P2G] dimer, SDF1 P2G, T134, T140, T22, TC 14012, burixafor, USL311, viral macrophage inflammatory protein-II, WZ811, [64Cu]-AMD3100, [64Cu]-AMD3465, [68Ga] pentixafor, [90Y] pentixather, [99mTc] 02-AMD3100, [177Lu] pentixather, and 508MC1 Compound 26.

101. The method of claim 90, wherein the CXCR4 inhibitor is selected from burixafor, plerixafor, and ulocuplumab.

102. The method of claim 90, wherein the CXCR4 inhibitor is burixafor.

103. The method of claim 90, wherein the ADRB2 inhibitor is selected from the group consisting of alprenolol, atenolol, betaxolol, bupranolol, butoxamine, carazolol, carvedilol, CGP 12177, cicloprolol, ICI 118551, ICYP, labetalol, levobetaxolol, levobunolol, LK 204-545, metoprolol, nadolol, NIHP, NIP, propafenone, propranolol, sotalol, SR59230A, and timolol.

104. The method of claim 90, wherein the ADRB2 inhibitor is propranolol.

105. The method of claim 90, wherein the CXCR4 inhibitor is selected from the group consisting of burixafor, plerixafor, and ulocuplumab, and wherein the ADRB2 inhibitor is propranolol.

106. The method of claim 90, wherein the CXCR4 inhibitor is burixafor, and wherein the ADRB2 inhibitor is propranolol.

107. The method of claim 90, wherein the method further comprises administering G-CSF to the subject.

108. The method of claim 90, wherein the method does not comprise administering G-CSF to the subject.

109. The method of claim 90, wherein the CXCR4 inhibitor is burixafor, wherein the ADRB2 inhibitor is propranolol, wherein the cancer is multiple myeloma, and wherein the treatment further comprises administration of G-CSF.

110. The method of claim 90, wherein the CXCR4 inhibitor is burixafor, wherein the ADRB2 inhibitor is propranolol, wherein the cancer is multiple myeloma, and wherein the treatment does not comprise administration of G-CSF.

111. The method of claim 90, wherein the CXCR4 inhibitor is burixafor, wherein the ADRB2 inhibitor is propranolol, wherein the cancer is multiple myeloma, wherein the stem cell transplantation comprises autologous stem cell transplantation, and wherein the treatment comprises administration of G-CSF.

112. The method of claim 90, wherein the CXCR4 inhibitor is burixafor, wherein the ADRB2 inhibitor is propranolol, wherein the cancer is multiple myeloma, wherein the stem cell transplantation comprises autologous stem cell transplantation, and wherein the treatment does not comprise administration of G-CSF.

113. The method of claim 90, wherein the CXCR4 inhibitor is burixafor, wherein the ADRB2 inhibitor is propranolol, wherein the cancer is multiple myeloma, wherein the stem cell transplantation comprises allogeneic stem cell transplantation, and wherein the treatment comprises administration of G-CSF.

114. The method of claim 90, wherein the CXCR4 inhibitor is burixafor, wherein the ADRB2 inhibitor is propranolol, wherein the cancer is multiple myeloma, wherein the stem cell transplantation comprises allogeneic stem cell transplantation, and wherein the treatment does not comprise administration of G-CSF.

115. A composition or compositions for treatment of cancer comprising, alone or in combination, a CXCR4 inhibitor and a beta-2-adrenergic receptor (ADRB2) inhibitor, wherein the treatment comprises stem cell transplantation.

116. The composition or compositions of claim 115, wherein the treatment comprises hematopoietic stem cell transplantation or hematopoietic progenitor cell transplantation.

117. The composition or compositions of claim 115, wherein the stem cell transplantation comprises autologous stem cell transplantation.

118. The composition or compositions of claim 115, wherein the stem cell transplantation comprises allogeneic stem cell transplantation.

119. The composition or compositions of claim 115, wherein the cancer is selected from lymphoma, leukemia, or myeloma.

120. The composition or compositions of claim 115, wherein the cancer is selected from non-Hodgkin lymphoma, acute myeloid leukemia, acute lymphoblastic leukemia, or multiple myeloma.

121. The composition or compositions of claim 115, wherein the cancer is multiple myeloma.

122. The composition or compositions of claim 115, wherein the CXCR4 inhibitor is selected from the group consisting of ALX40-4C, AMD070, plerixafor, AMD3465, ATI 2341, BKT140, CTCE-9908, CX549, D-[Lys3] GHRP-6, FC122, FC131, GMI-1359, GSK812397, GST-NT21MP, isothiourea-la, isothiourea-It (IT1t), KRH-1636, KRH-3955, LY2510924, MSX-122, N-[11C] Methyl-AMD3465, POL6326, SDF-1 1-9 [P2G] dimer, SDF1 P2G, T134, T140, T22, TC 14012, burixafor, USL311, viral macrophage inflammatory protein-II, WZ811, [64Cu]-AMD3100, [64Cu]-AMD3465, [68Ga] pentixafor, [90Y] pentixather, [99mTc] 02-AMD3100, [177Lu] pentixather, and 508MC1 Compound 26.

123. The composition or compositions of claim 115, wherein the CXCR4 inhibitor is selected from burixafor, plerixafor, and ulocuplumab.

124. The composition or compositions of claim 115, wherein the CXCR4 inhibitor is burixafor.

125. The composition or compositions of claim 115, wherein the ADRB2 inhibitor is selected from the group consisting of alprenolol, atenolol, betaxolol, bupranolol, butoxamine, carazolol, carvedilol, CGP 12177, cicloprolol, ICI 118551, ICYP, labetalol, levobetaxolol, levobunolol, LK 204-545, metoprolol, nadolol, NIHP, NIP, propafenone, propranolol, sotalol, SR59230A, and timolol.

126. The composition or compositions of claim 115, wherein the ADRB2 inhibitor is propranolol.

127. The composition or compositions of claim 115, wherein the CXCR4 inhibitor is selected from the group consisting of burixafor, plerixafor, and ulocuplumab, and wherein the ADRB2 inhibitor is propranolol.

128. The composition or compositions of claim 115, wherein the CXCR4 inhibitor is burixafor, and wherein the ADRB2 inhibitor is propranolol.

129. The composition or compositions of claim 115, wherein the CXCR4 inhibitor is burixafor, wherein the ADRB2 inhibitor is propranolol, wherein the cancer is multiple myeloma, and wherein the composition or compositions further comprise G-CSF.

130. The composition or compositions of claim 115, wherein the CXCR4 inhibitor is burixafor, wherein the ADRB2 inhibitor is propranolol, wherein the cancer is multiple myeloma, and wherein the composition or compositions do not comprise G-CSF.

131. The composition or compositions of claim 115, wherein the CXCR4 inhibitor is burixafor, wherein the ADRB2 inhibitor is propranolol, wherein the cancer is multiple myeloma, wherein the stem cell transplantation comprises autologous stem cell transplantation, and wherein the composition or compositions further comprise G-CSF.

132. The composition or compositions of claim 115, wherein the CXCR4 inhibitor is burixafor, wherein the ADRB2 inhibitor is propranolol, wherein the cancer is multiple myeloma, wherein the stem cell transplantation comprises autologous stem cell transplantation, and wherein the composition or compositions do not comprise G-CSF.

133. The composition or compositions of claim 115, wherein the CXCR4 inhibitor is burixafor, wherein the ADRB2 inhibitor is propranolol, wherein the cancer is multiple myeloma, wherein the stem cell transplantation comprises allogeneic stem cell transplantation, and wherein the composition or compositions further comprise G-CSF.

134. The composition or compositions of claim 115, wherein the CXCR4 inhibitor is burixafor, wherein the ADRB2 inhibitor is propranolol, wherein the cancer is multiple myeloma, wherein the stem cell transplantation comprises allogeneic stem cell transplantation, and wherein the composition or compositions do not comprise G-CSF.

135. A pharmaceutical composition or pharmaceutical compositions for treatment of cancer comprising, alone or in combination, a CXCR4 inhibitor and a beta-2-adrenergic receptor (ADBR2), and a pharmaceutically acceptable excipient, wherein the treatment comprises stem cell transplantation.

136. The pharmaceutical composition or compositions of claim 135, wherein the treatment comprises hematopoietic stem cell transplantation or hematopoietic progenitor cell transplantation.

137. The pharmaceutical composition or compositions of claim 135, wherein the stem cell transplantation comprises autologous stem cell transplantation.

138. The pharmaceutical composition or compositions of claim 135, wherein the stem cell transplantation comprises allogeneic stem cell transplantation.

139. The pharmaceutical composition or compositions of claim 135, wherein the cancer is selected from lymphoma, leukemia, or myeloma.

140. The pharmaceutical composition or compositions of claim 135, wherein the cancer is selected from non-Hodgkin lymphoma, acute myeloid leukemia, acute lymphoblastic leukemia, or multiple myeloma.

141. The pharmaceutical composition or compositions of claim 135, wherein the cancer is multiple myeloma.

142. The pharmaceutical composition or compositions of claim 135, wherein the CXCR4 inhibitor is selected from the group consisting of ALX40-4C, AMD070, plerixafor, AMD3465, ATI 2341, BKT140, CTCE-9908, CX549, D-[Lys3] GHRP-6, FC122, FC131, GMI-1359, GSK812397, GST-NT21MP, isothiourea-la, isothiourea-It (IT1t), KRH-1636, KRH-3955, LY2510924, MSX-122, N-[11C] Methyl-AMD3465, POL6326, SDF-1 1-9 [P2G] dimer, SDF1 P2G, T134, T140, T22, TC 14012, burixafor, USL311, viral macrophage inflammatory protein-II, WZ811, [64Cu]-AMD3100, [64Cu]-AMD3465, [68Ga] pentixafor, [90Y] pentixather, [99mTc] 02-AMD3100, [177Lu] pentixather, and 508MC1 Compound 26.

143. The pharmaceutical composition or compositions of claim 135, wherein the CXCR4 inhibitor is selected from burixafor, plerixafor, and ulocuplumab.

144. The pharmaceutical composition or compositions of claim 135, wherein the CXCR4 inhibitor is burixafor.

145. The pharmaceutical composition or compositions of claim 135, wherein the ADRB2 inhibitor is selected from the group consisting of alprenolol, atenolol, betaxolol, bupranolol, butoxamine, carazolol, carvedilol, CGP 12177, cicloprolol, ICI 118551, ICYP, labetalol, levobetaxolol, levobunolol, LK 204-545, metoprolol, nadolol, NIHP, NIP, propafenone, propranolol, sotalol, SR59230A, and timolol.

146. The pharmaceutical composition or compositions of claim 135, wherein the ADRB2 inhibitor is propranolol.

147. The pharmaceutical composition or compositions of claim 135, wherein the CXCR4 inhibitor is selected from the group consisting of burixafor, plerixafor, and ulocuplumab, and wherein the ADRB2 inhibitor is propranolol.

148. The pharmaceutical composition or compositions of claim 135, wherein the CXCR4 inhibitor is burixafor, and wherein the ADRB2 inhibitor is propranolol.

149. The pharmaceutical composition or compositions of claim 135, wherein the CXCR4 inhibitor is burixafor, wherein the ADRB2 inhibitor is propranolol, wherein the cancer is multiple myeloma, and wherein the pharmaceutical composition or pharmaceutical compositions further comprise G-CSF.

150. The pharmaceutical composition or compositions of claim 135, wherein the CXCR4 inhibitor is burixafor, wherein the ADRB2 inhibitor is propranolol, wherein the cancer is multiple myeloma, and wherein the pharmaceutical composition or pharmaceutical compositions do not comprise G-CSF.

151. The pharmaceutical composition or compositions of claim 135, wherein the CXCR4 inhibitor is burixafor, wherein the ADRB2 inhibitor is propranolol, wherein the cancer is multiple myeloma, wherein the stem cell transplantation comprises autologous stem cell transplantation, and wherein the pharmaceutical composition or pharmaceutical compositions further comprise G-CSF.

152. The pharmaceutical composition or compositions of claim 135, wherein the CXCR4 inhibitor is burixafor, wherein the ADRB2 inhibitor is propranolol, wherein the cancer is multiple myeloma, wherein the stem cell transplantation comprises autologous stem cell transplantation, and wherein the pharmaceutical composition or pharmaceutical compositions do not comprise G-CSF.

153. The pharmaceutical composition or compositions of claim 135, wherein the CXCR4 inhibitor is burixafor, wherein the ADRB2 inhibitor is propranolol, wherein the cancer is multiple myeloma, wherein the stem cell transplantation comprises allogeneic stem cell transplantation, and wherein the pharmaceutical composition or pharmaceutical compositions further comprise G-CSF.

154. The pharmaceutical composition or compositions of claim 135, wherein the CXCR4 inhibitor is burixafor, wherein the ADRB2 inhibitor is propranolol, wherein the cancer is multiple myeloma, wherein the stem cell transplantation comprises allogeneic stem cell transplantation, and wherein the pharmaceutical composition or pharmaceutical compositions do not comprise G-CSF.

155. A method of mobilizing a cell in a subject comprising administering, alone or in combination, a CXCR4 inhibitor and a beta-2-adrenergic receptor (ADRB2) inhibitor to a subject.

156. The method of claim 155, wherein the administering comprises administering the ADRB2 inhibitor and the CXCR4 inhibitor simultaneously, concurrently, or sequentially to the subject.

157. The method of claim 155, wherein the administering comprises administering the ADRB2 inhibitor to the subject before administering the CXCR4 inhibitor to the subject.

158. The method of claim 155, wherein administering the ADRB2 inhibitor is initiated at a first specific time interval before administering the CXCR4 inhibitor, and wherein the first specific time interval is 6 days to 7 days.

159. The method of claim 155, wherein the cell is a stem cell.

160. The method of claim 155, wherein the cell is a stem cell, and wherein the mobilizing is used for treatment of cancer, and wherein the treatment comprises stem cell transplantation.

161. The method of claim 160, wherein the treatment comprises hematopoietic stem cell transplantation or hematopoietic progenitor cell transplantation.

162. The method of claim 160, wherein the stem cell transplantation comprises autologous stem cell transplantation.

163. The method of claim 160, wherein the stem cell transplantation comprises allogeneic stem cell transplantation.

164. The method of claim 160, wherein the cancer is selected from lymphoma, leukemia, and myeloma.

165. The method of claim 160, wherein the cancer is selected from non-Hodgkin lymphoma, acute myeloid leukemia, acute lymphoblastic leukemia, and multiple myeloma.

166. The method of claim 160, wherein the cancer is multiple myeloma.

167. The method of claim 155, wherein the CXCR4 inhibitor is selected from the group consisting of ALX40-4C, AMD070, plerixafor, AMD3465, ATI 2341, BKT140, CTCE-9908, CX549, D-[Lys3] GHRP-6, FC122, FC131, GMI-1359, GSK812397, GST-NT21MP, isothiourea-la, isothiourea-It (IT1t), KRH-1636, KRH-3955, LY2510924, MSX-122, N-[11C] Methyl-AMD3465, POL6326, SDF-1 1-9 [P2G] dimer, SDF1 P2G, T134, T140, T22, TC 14012, burixafor, USL311, viral macrophage inflammatory protein-II, WZ811, [6Cu]-AMD3100, [64Cu]-AMD3465, [68Ga] pentixafor, [90Y] pentixather, [^{99m}Tc] 02-AMD3100, [177Lu] pentixather, and 508MC1 Compound 26.

168. The method of claim 155, wherein the CXCR4 inhibitor is selected from burixafor, plerixafor, and ulocuplumab.

169. The method of claim 155, wherein the CXCR4 inhibitor is burixafor.

170. The method of claim 155, wherein the ADRB2 inhibitor is selected from the group consisting of alprenolol, atenolol, betaxolol, bupranolol, butoxamine, carazolol, carvedilol, CGP 12177, cicloprolol, ICI 118551, ICYP, labetalol, levobetaxolol, levobunolol, LK 204-545, metoprolol, nadolol, NIHP, NIP, propafenone, propranolol, sotalol, SR59230A, and timolol.

171. The method of claim 155, wherein the ADRB2 inhibitor is propranolol.

172. The method of claim 155, wherein the CXCR4 inhibitor is selected from the group consisting of burixafor, plerixafor, and ulocuplumab, and wherein the ADRB2 inhibitor is propranolol.

173. The method of claim 155, wherein the CXCR4 inhibitor is burixafor, and wherein the ADRB2 inhibitor is propranolol.

174. The method of claim 155, wherein the method further comprises administering G-CSF to the subject.

175. The method of claim 155, wherein the method does not comprise administering G-CSF to the subject.

176. The method of claim 160, wherein the CXCR4 inhibitor is burixafor, wherein the ADRB2 inhibitor is propranolol, wherein the cancer is multiple myeloma, and wherein the treatment further comprises administration of G-CSF.

177. The method of claim 160, wherein the CXCR4 inhibitor is burixafor, wherein the ADRB2 inhibitor is propranolol, wherein the cancer is multiple myeloma, and wherein the treatment does not comprise administration of G-CSF.

178. The method of claim 160, wherein the CXCR4 inhibitor is burixafor, wherein the ADRB2 inhibitor is propranolol, wherein the cancer is multiple myeloma, wherein the stem cell transplantation comprises autologous stem cell transplantation, and wherein the treatment comprises administration of G-CSF.

179. The method of claim 160, wherein the CXCR4 inhibitor is burixafor, wherein the ADRB2 inhibitor is propranolol, wherein the cancer is multiple myeloma, wherein the stem cell transplantation comprises autologous stem cell transplantation, and wherein the treatment does not comprise administration of G-CSF.

180. The method of claim 160, wherein the CXCR4 inhibitor is burixafor, wherein the ADRB2 inhibitor is propranolol, wherein the cancer is multiple myeloma, wherein the stem cell transplantation comprises allogeneic stem cell transplantation, and wherein the treatment comprises administration of G-CSF.

181. The method of claim 160, wherein the CXCR4 inhibitor is burixafor, wherein the ADRB2 inhibitor is propranolol, wherein the cancer is multiple myeloma, wherein the stem cell transplantation comprises allogeneic stem cell transplantation, and wherein the treatment does not comprise administration of G-CSF.
